SEQUENCE LISTING (24 sequences)

SEQ ID NO: 1 - DNA, 1208 bp, Homo sapiens
  CDS (31)..(870)
gagcgggagtggagggcccagtccctacccatggcagtagggggcgttttgaagctgcggctctgtgagctgtggctactgctactgggttctagtttgaacgccagatttttgccagacgaggaggacgtagactttatcaacgagtacgtgaacctccacaatgagctgcggggcgacgttattccccgagggtctaacttgcgcttcatgacttgggatgtagctttatcacggactgctagagcatggggaaaaaaatgtttgtttacgcataatatttatttacaagatgtacaaatggtccatcctaaattttatggtattggtgaaaatatgtgggtcggccctgaaaatgaatttactgcaagtattgctatcagaagttggcatgcagagaagaaaatgtacaattttgaaaatggcagttgctctggagactgttctaattatattcagcttgtttgggaccactcttacaaagttggttgtgctgttactccatgttcaaaaattggacatattatacatgcagcaattttcatatgcaactatgcgccaggaggaacactgacgagaagaccttatgaaccaggaatattttgtactcgatgtggcagacgtgacaaatgcacagattttctatgcaagtcttgctctgtcgccaggctggagtgtaatcgcaccatcttggctcactgcaacctccgcctccagggttcaagtgattatcctgcctcagcctcccaagtagctgagactacaggtaatgcagatcgtgaccaagccacatattaccgattttggtatccaaaatgggaaatgccccggccagttgtgtgtgatccactgtgccacattcaagcttaatattgagaatattatgttttatcctgtgtgtcataactgttttgatagtacagtctcagtttccaaatatcttgttggaacaacaaatgatatttacccctgaggaatctgaagcagggaatgaagaggaggaaaaagaggaagagaagaaagagaaagaggaaatggaaatggaaataatggaaatggaggaggaaaaagaagagagagaggaggaggaggaggaaacacaaaaagaaaagatggaggaagaggaaaaataagagtagaaagaggaggaaaaagatgtatcaccaatataaaccaaaagtgtaatacaaaaaaagacagaaaaaaaa

SEQ ID NO: 2 - protein, 279 aa, Homo sapiens
MAVGGVLKLRLCELWLLLLGSSLNARFLPDEEDVDFINEYVNLHNELRGDVIPRGSNLRFMTWDVALSRTARAWGKKCLFTHNIYLQDVQMVHPKFYGIGENMWVGPENEFTASIAIRSWHAEKKMYNFENGSCSGDCSNYIQLVWDHSYKVGCAVTPCSKIGHIIHAAIFICNYAPGGTLTRRPYEPGIFCTRCGRRDKCTDFLCKSCSVARLECNRTILAHCNLRLQGSSDYPASASQVAETTGNADRDQATYYRFWYPKWEMPRPVVCDPLCHIQA

SEQ ID NO: 3 - DNA, 3470 bp, Homo sapiens
  CDS (10)..(1401)
ctggccaccatgcacggctcctgcagtttcctgatgcttctgctgccgctactgctactgctggtggccaccacaggccccgttggagccctcacagatgaggagaaacgtttgatggtggagctgcacaacctctaccgggcccaggtatccccgccggcctcagacatgctgcacatgagatgggacgaggagctggccgccttcgccaaggcctacgcacggcagtgcgtgtggggccacaacaaggagcgcgggcgccgcggcgagaatctgttcgccatcacagacgagggcatggacgtgccgctggccatggaggagtggcaccacgagcgtgagcactacaacctcagcgccgccacctgcagcccaggccagatgtgcggccactacacgcaggtggtatgggccaagacagagaggatcggctgtggttcccacttctgtgagaagctccagggtgttgaggagaccaacatcgaattactggtgtgcaactatgagcctccggggaacgtgaaggggaaacggccctaccaggaggggactccgtgctcccaatgtccctctggctaccactgcaagaactccctctgtgaacccatcggaagcccggaagatgctcaggatttgccttacctggtaactgaggccccatccttccgggcgactgaagcatcagactctaggaaaatgggtactccttcttccctagcaacggggattccggctttcttggtaacagaggtctcaggctccctggcaaccaaggctctgcctgctgtggaaacccaggccccaacttccttagcaacgaaagacccgccctccatggcaacagaggctccaccttgcgtaacaactgaggtcccttccattttggcagctcacagcctgccctccttggatgaggagccagttaccttccccaaatcgacccatgttcctatcccaaaatcagcagacaaagtgacagacaaaacaaaagtgccctctaggagcccagagaactctctggaccccaagatgtccctgacaggggcaagggagctcctaccccatgcccaggaggaggctgaggctgaggctgagttgcctccttccagtgaggtcttggcctcagtttttccagcccaggacaagccaggtgagctgcaggccacactggaccacacggggcacacctcctccaagtccctgcccaatttccccaatacctctgccaccgctaatgccacgggtgggcgtgccctggctctgcagtcgtccttgccaggtgcagagggccctgacaagcctagcgtcgtgtcagggctgaactcgggccctggtcatgtgtggggccctctcctgggactactgctcctgcctcctctggtgttggctggaatcttctgaaggggataccactcaaaggcaaggcctggtgaggggggccctggcctcatacccacctggattgtcttcctccaagtgagagaccacagcttcctgggcaggtcctgctctgtggcccagcagccccccttcaccccaacttctggccagattccaggccagcactcttgtcctcctgggaggcgtctacagggccagcccctggcactgccccaggagtgccttggctctgggtaggcccatccttcagctggctgcagactgttctgagcgctatttacatgtgcccactctcaggttgtcctgtggccatcagcttctctcccagacagaggatctcaggcttcccaggaacccccgggcccctcccagtcccctggcctcttccttgagccatctgagtccaggactgttccccagaagtgcctcttgccttctcagggtgaagaggtcagctgtcctcctgtcatcttccccaccctgtccccagcccctaaacaagatacttcttggttaaggccctccggaaagggaaaggctacggggcatgtgcctcatcacaccatccatcctggaggcacaaggcctggctggctgcgagctcagggggccgcctgaggactgcacaccgggcccacacctctcctgcccctccctcctgagtcctgggggtgggaggatttgagggagctcactgcctacctggcctggggctgtctgcccacacagcatgtgcgctctccctgagtgcctgtgtagctggggatggggattcctaggggcagatgaaggacaagccccactggagtggggttctttgagtgggggaggcagggacgaggaaggaaagcaactcctgaatctccaataaaaactgtccaacctgtggcaactattccttctctttctcatccccctcctctaaaacactccatttagtgtctcctctttgggaccacccttcatttccttcctataactctcgcctccccctaatcacaggtggcaggcaggaaggggaagagccaaaggaaggccaagggtgctgggctgactcccccagcttcaggagtcaccagtggggcctaggtaggtttggagcccaggatcaaagcatggcatgcggcctcttcacgggggcgctgctatccaggcctgtctgggacacaaggtcagaaatgtacatcaggcaaggctctgccagccccaggtgcttggaagagtcctaagaaagggatctggagtcccagaccctccccagggggagcaaagcaggactgtccagaggtgctgaggatcaggaaagcacacatatgctggggtagcagtggtggatgactgagggtggcttgggtggtagggaggggcagagcaagtctccagaccaccgctggaaggtgagcagtcaaacaggacaccacccaagtcaggccaggggctgggaaccgatctgtaaccaaggaggacactcccctcttttactttcttttttttttttttgagatagagttttgctttgtcgccccaggctggagtgaagtgatctcggctcactgcaacctccgcccaccgggttcaagcgattctcctgcctcagcctcccgagtagctgggattataggcacctgccaacacgcccagctaatttttgtatttttagtagagacggggtttggtcaacaggtggtcttgaactcctgacctcaggtgatccaccggcttcagcctcccaaagtgctaggattacgggtgtgagccaccacgcccggtctactctatgttttttccagctctattcagatataactcacaacataaaattcacccatttaaaatatgcaactcaacggtttttactatattcacaaggttgtgtaaaaccgtcacaattttagaacattttcatcaccccaaaaagaaaccccatacccattagcagtcactccctatttccctccaacctccccagccccaatcaaccactaatctattttctatctatatggatttgtctattcttaacatccatataaattcttgacatatcataaaaaaaaaaaaaaaaaaaa

SEQ ID NO: 4 - protein, 463 aa, Homo sapiens [sequence omitted]

SEQ ID NO: 5 - protein, 265 aa, Ancylostoma caninum
IAIAGIAHSNEHNLRCPQNGTEMPGFNDSIRLQFLAMHNGYRSKLALGHISITEESESDDDDDFGFLPDFAPRASKMRYLEYDCEAEKSAYMSARNCSDSSSPPEGYDENKYIFENSNNISEAALKAMISWAKEAFNLNKTKEGEGVLYRSNHDISNFANLAWDAREKFGCAVVNCPLGEIDDETNHDGETYATTIHVVCHYPKINKTEGQPIYKVGTPCDDCSEYTKKADNTTSADPVCIPDDGVCFIGSKADYDSKEFYRFRE

SEQ ID NO: 6 - protein, 274 aa, Homo sapiens
MEAYLVVLIAIAGIAHSNEHNLRCPQNGTEMPGFNDSIRLQFLAMHNGYRSKLALGHISITEESESDDDDDFGFLPDFAPRASKMRYLEYDCEAEKSAYMSARNCSDSSSPPEGYDENKYIFENSNNISEAALKAMISWAKEAFNLNKTKEGEGVLYRSNHDISNFANLAWDAREKFGCAVVNCPLGEIDDETNHDGETYATTIHVVCHYPKINKTEGQPIYKVGTPCDDCSEYTKKADNTTSADPVCIPDDGVCFIGSKADYDSKEFYRFREL

SEQ ID NO: 7 - protein, 219 aa, Homo sapiens
MVSFVSNYSHTANILPDIENEDFIKDCVRIHNKFRSEVKPTASDMLYMTWDPALAQIAKAWASNCQFSHNTRLKPPHKLHPNFTSLGENIWTGSVPIFSVSSAITNWYDEIQDYNFKTRICKKVCGHYTQVVWADSYKVGCAVQFCPKVSGFDALSNGAHFICNYGPGGNYPTWPYKRGATCSACPNNDKCLDNLCVNDSETKSNVTTMLYIRLAHIST

SEQ ID NO: 8 - DNA, 371 bp, Homo sapiens
ccagcccctaaacaagatacttcttggttaaggccctccggaagggaaaggctacggggcatgtgcctcatcacaccatccatcctggaggcacaaggcctggtggctgcgagctcaggaggccgcctgaggactgcacaccgggccacacctctcctgcccctccctcctgagtcctgggggtgggaggatttgagggagctcactgcctacctggcctggggctgttgcccacacagcatgtgcgctctccctgagtgcctgtttagctggggatggggattcctaggggcagatgaaggacaagccccactggagtggggttcttttaagtgggggaggcagggacgagggaaggaaagtaaatcctt

SEQ ID NO: 9 - DNA, 248 bp, Homo sapiens
cctctccgcccctccctcctgagtcctgggggtgggaggatttgagggagctcactgcctacctggcctgggctgtctgcccacacagcatgtgcgctctccctgagtgccttgtagctggggatggggattcctaggggcagatgaaggacaagccccactggagtggggttctttgagtgggggaggcagggacgagggaaggaaagcaactcctactctccaataaaaacctgtccaacctgtgg

SEQ ID NO: 10 - DNA, 472 bp, Homo sapiens
aattcggcaagtccttccgggcgactgaagcatcagactctaggaaaatgggtgcagaggcctgacaagcctagcgtcgtgtcagggctgaactcgggcctggtcatgtgtggggccctctcctgggactactgctcctgcctcctctggtgttggctggaatcttctgaagggataccactcaaaggcaaggctggtgaggggggccctggcctcatacccacctggattgtttcctccatgagagaccacattcctggggaagtcctgttttgggcccaagcccctttaacccaaattttggccaattccagccaaatttttctctggggggtttaaaggccacctggaattccccagagtcttggttttggaggccattttaaatggttaaattttttggggttttaaaagccattggttctggaaattttttcaaaaggttagtttaggacccgggctaatccggttt

SEQ ID NO: 11 - DNA, 310 bp, Homo sapiens
gacaagcctagcgtcgtgtcagggctgaactcgggccctggtcatgttggggccctctcctgggactactgctcctgcctcctttggtgttggctggaatcttctgaaggggataccactcaaagggtgaagaggtcagctgtcctcctgtcatcttccccaccctgtccccagcccctaaacaagatacttcttggttaaggccctccggaagggaaaggctacggggcatgtgcctcatcacaccatccatcctggaggcacaaggcctggctggctgcgagctcaggaggccgcctgaggactgcaa

SEQ ID NO: 12 - DNA, 25 bp, Artificial sequence
  Contains an NcoI restriction site
cgcccatggccagatttttgccaga

SEQ ID NO: 13 - DNA, 24 bp, Artificial sequence
  Contains a HindIII restriction site
cgcaagcttgaatgtggcacagtg

SEQ ID NO: 14 - DNA, 27 bp, Artificial sequence
  Contains a BamH1 restriction site
cgcggatccggccccgttggagccctc

SEQ ID NO: 15 - DNA, 33 bp, Artificial sequence
  Contains a HindIII restriction site
cggggtaccaagctttcagccccacacatgacc

SEQ ID NO: 16 - DNA, 36 bp, Artificial sequence
  Contains a BamH1 restriction site
cgcggatccgccatcatggcagtagggggcgttttg

SEQ ID NO: 17 - DNA, 24 bp, Artificial sequence
  Contains a Asp718 restriction site
cgcggtaccgaatgtggcacagtg

SEQ ID NO: 18 - DNA, 32 bp, Artificial sequence
  Contains a BamH1 restriction site
cgcggatccgccatcatgcacggctcctgcag

SEQ ID NO: 19 - DNA, 26 bp, Artificial sequence
  Contains a Asp718 restriction site
cgcggtaccgtctctcacttggagga

SEQ ID NO: 20 - DNA, 33 bp, Artificial sequence
  Contains a Asp718 restriction site
cgcggtaccaagctttcagccccacacatgacc

SEQ ID NO: 21 - protein, 11 aa, Homo sapiens
GHYTQVVWAKT

SEQ ID NO: 22 - protein, 12 aa, Homo sapiens
LLVCNYEPPGNV

SEQ ID NO: 23 - protein, 11 aa, Homo sapiens
EVPSILAAHSL

SEQ ID NO: 24 - protein, 16 aa, Homo sapiens
PGHVMGPLLGLLLLPP

(19) United States
(12) Patent Application Publication
(10) Pub. No.: US 2003/0162956 A1
(43) Pub. Date: Aug. 28, 2003

(54) Leukocyte regulatory factors 1 and 2

(75) Inventors: Jian Ni, Germantown, MD (US); Jing-Shan Hu, Mountain View, CA (US); Steven M. Ruben, Brookeville, MD (US); Reiner L. Gentz, Belo Horizonte-Mg (BR)

Correspondence Address: HUMAN GENOME SCIENCES INC, 9410 KEY WEST AVENUE, ROCKVILLE, MD, 20850

(73) Assignee: Human Genome Sciences, Inc., Rockville, MD (US)

(21) Appl. No.: 10/387,495

(22) Filed: Mar. 14, 2003

Related U.S. Application Data
(63) Continuation of application No. 09/603,735, filed on Jun. 23, 2000.
Application No. 09/603,735 is a continuation of application No. 09/055,998, filed on Apr. 7, 1998, now abandoned.
(60) Provisional application No. 60/043,483, filed on Apr. 7, 1997.

(51) Int. Cl. C07H 21/04

(52) U.S. Cl. 536/23.5

(57) ABSTRACT

The present invention relates to novel LRF-1 and LRF-2 proteins which are related to the CRISP family and a protein called “Neutrophil Inhibitory Factor (NIF)” isolated from the canine hookworm (Ancylostoma caninum) that potently inhibits CD11/CD18-dependent neutrophil function. In particular, isolated nucleic acid molecules are provided encoding the human LRF-1 and LRF-2 proteins. LRF-1 and LRF-2 polypeptides are also provided, as are vectors, host cells and recombinant methods for producing the same. The invention further relates to screening methods for identifying agonists and antagonists of LRF-1 or LRF-2 activity. Also provided are diagnostic methods for detecting immune system or other LRF-1 or LRF-2-related disorders and therapeutic methods for treating such disorders.

This application is a continuation of U.S. application Ser. No. 09/603,735, filed Jun. 23, 2000, which is a continuation of U.S. application Ser. No. 09/055,998, filed Apr. 7, 1998 (now abandoned), which claims the benefit under 35 U.S.C. §119(e) of U.S. Provisional Application No. 60/043,483, filed Apr. 7, 1997; each of the above applications is hereby incorporated by reference in its entirety.

### FIELD OF THE INVENTION

The present invention relates to genes encoding novel human members of a family of secreted proteins which exhibit a variety of defense functions including antifungal, antibacterial, antiviral, and antiparasite activities as well as modulation of immune system functions, particularly functions of polymorphonuclear leukocytes (neutrophils). More specifically, isolated nucleic acid molecules are provided encoding human polypeptides, named Leukocyte Regulatory Factor-1 and Leukocyte Regulatory Factor-2, hereinafter referred to, respectively, as LRF-1 and LRF-2. LRF-1 and LRF-2 polypeptides are also provided, as are vectors, host cells and recombinant methods for producing the same. Also provided are diagnostic methods for detecting disorders related to the immune system, and therapeutic methods for treating such disorders. The invention further relates to screening methods for identifying agonists and antagonists of LRF-1 or LRF-2 activity.

### BACKGROUND OF THE INVENTION

Pathogenesis-Related (PR) Proteins are known to be produced by many plant species in response to infection by pathogenic viruses, bacteria and fungi. See, for instance, Rigden, J. and Coutts, R., Trends Genet. 4:87-89 (1988). Several of these proteins possess antifungal activities in vitro and/or biochemical activities such as chitinase, glucanase, and permatin activities. For the class of PR proteins known as PR-1 proteins, consisting of about 130 to 140 residues and probably containing three disulfide bonds, no biochemical function has been demonstrated yet. However, like the PR proteins with enzymatic activities, they exist in both basic and acidic isoforms, which has led to the suggestion that the PR-1 proteins also have some as yet undefined enzymatic function. Cornelissen, B. J. C. et al., Nucleic Acids Res. 15:6799-6811 (1987). Most importantly, expression of PR-1a in transgenic tobacco mediates tolerance to certain fungal pathogens, demonstrating that PR-l a can act as defense protein. Alexander, D. et al, Proc. Natl. Acad. Sci. USA 90:7327-7331 (1990). Similar PR-proteins have been isolated from pathogen-infected tomato plants. See, for instance, Torero, P., et al., Mol Gen. Genet. 243.47-53 (1994). Thus, pathogen-induced proteins with inhibitory activity toward the fungus Phytophthora infestans, both in vitro (inhibition of zoospore germination) and in vivo with a tomato leaf disc assay (decrease in infected leaf surface) have been demonstrated. Woloshuk, C. P., et al., Plant Cell 3.619-628 (1991).

Certain mammalian proteins exhibit homology to plant PR proteins. For instance, several members of the cysteine-rich secretory protein (CRISP) family show such homology. In the mouse CRISP-1 is thought to be the counterpart of a previously discovered rat acidic epididymal glycoprotein (AEG) which has been shown to be attached to the plasma membrane at the sperm head. A cDNA for CRISP-3, which shows about 77% amino acid identity to the CRISP-1 protein, was isolated from a mouse salivary gland library by homology to a rat AEG cDNA. Haendler, B. J. et al., Endocrinology 133.192-198 (1993). Furthermore, CRISP-1 and CRISP-3 are 47% identical in amin acid sequence to the deduced sequence of the mouse testis-specific gene-encoded protein Tpx-1 (now CRISP-2), with which they share the conserved spacing of 16 cysteine residues in the carboxy-terminal half of the molecules. Mizuki. N., et al., Mamm. Genome 3.274-280 (1992). Recently, human TPX-1 has been recognized as a member of the human CRISP family. Kratzschmar, J. et al, Eur. J Biochem. 236.827-836 (1996). Other than in salivary glands, CRISP-3 was found to be expressed in mice only in lymphoid tissues, most highly in bone marrow and with somewhat reduced expression in spleen and significantly lower levels in thymus and lymph nodes. Pfisterer, P. et al., Mol. Cell. Biol. 16:6160-6168 (1996). Within lymphoid cells CRISP-3 expression was detected only in certain pre-B cells in the B cell lineage.

Helothermine, a toxin with hypothermic effects originating from the salivary secretions of the Mexican lizard Heloderma horridum, has recently been found to be another member of the CRISP family. Mochca-Morales, J. et al, Toxicon 28.299-309 (1990). Helothermine has been shown to block the ryanodine-sensitive sarcoplasmic calcium release channel in cell-free assays. In addition, CRISP sequences show some stretches of complete identity and an overall 30% identity to two groups of nonmammalian proteins, certain venom proteins of vespids and ants (e.g., venom sac proteins of white-face hornets, known as Dol m V), and to the plant PR proteins discussed above (for a detailed alignment of these proteins, see Morrisette, J. et al., Biophys. J 68.2280-2288 (1995)). All of these more distantly related proteins lack the cysteine-rich C-terminal region characteristic of the CRISP family.

Due to its homology to the plant defense proteins and its expression in B lymphocytes, it has been suggested recently that CRISP-3 is involved in fighting pathogens in mammals. Pfisterer, P. et al., supra. More particularly, it has been suggested that CRISPs may encode lytic enzymatic activities, which would be consistent with the observed association of AEG (CRISP-1) with the sperm head and presence of AA1 (CRISP-2) in the acrosome, where they could be involved in degrading egg structures during fertilization. In the case of CRISP-3 which is expressed in the salivary gland and in B cells, such lytic activities could be related to antifungal or antibacterial functions in saliva and in the blood or lymph. Id.

Neutrophil polymorphonuclear (PMN) leukocytes (“neutrophils”) are essential for host defense and also are integral to the initiation and propagation of the acute inflammatory response. In reaction to early events during invasion of a pathogen or an inflammatory insult, they initially are activated to by chemotactic signals and respond by migrating through the circulatory system to the site of the insult. There they leave the capillaries to enter the affected tissue by a complex process involving margination (flowing nearer to the endothelial lining of blood vessels, rolling and then attaching), following which they emigrate between the endothelial cells (extravasation, or diapedesis). Several mediators are involved, including substances produced by micro-organisms, and by cells participating in the inflammatory process.

More in particular, activation of neutrophils evokes initiation of several specific effector functions: chemotaxis, phagocytosis, generation of toxic oxygen metabolites and degranulation. At the site of an acute inflammatory process, neutrophils kill microorganisms, release substances that modify the local and systemic inflammatory responses and secrete enzymes that aid in tissue remodeling. Untimely release of toxic neutrophil products (e.g., hydrogen peroxide) may cause damage to host tissues and is likely to contribute to the pathogenesis of some common and important human diseases (inflammatory arthritis, emphysema and coronary vascular ischemic syndromes, among others). A carefully regulated system of cellular recruitment and activation and termination therefore, is essential to optimize neutrophil antimicrobial effects while minimizing host tissue damage.

Cell adhesion molecules (CAMs) are cell surface proteins involved in the binding of cells, usually leukocytes such as neutrophils, to each other, to endothelial cells, or to extracellular matrix. Specific signals produced in response to wounding and infection control the expression and activation of certain of these adhesion molecules. The interactions and responses then initiated by binding of these CAMs to their receptors/ligands play important roles in the mediation of the inflammatory and immune reactions that constitute a major line of the body's defense against these insults. Most of the CAMs characterized so far fall into three general families of proteins: the immunoglobulin (Ig) superfamily, the integrin family, or the selectin family.

The integrins are heterodimeric proteins consisting of an alpha and a beta chain that mediate leukocyte adherence to the vascular endothelium or other cell-cell interactions. Different sets of integrins are expressed by different populations of leukocytes to provide specificity for binding to different types of CAMs expressed along the vascular endothelium. Neutrophils are attracted from the blood to a site of inflammation by a process that begins with a loose capture (and rolling in shear flow) of the cell by selectin-ligand interactions between the neutrophil and an endothelial cell. This brings the neutrophil in proximity with chemoattractants from the site of inflammation; the chemoattractants activate integrins and confer direction, both of which aid in the migration of the neutrophil across the endothelium to the inflamed site. In particular, activation of the beta 2 integrin CR3 (CD11b/CD18) plays an important role in inducing neutrophil functions involved in inflammation and anti-infection immune responses.

The chronic survival of many endoparasites is dependent on the ability of these organisms to escape the host immune response. Recently, the discovery of a glycoprotein that inhibits neutrophil function and is a ligand of the integrin CD11b/CD18 has been reported. Moyle, M., et al., J. Biol. Chem. 269: 10008-10015 (1994). This factor, called “Neutrophil Inhibitory Factor (NIF),” is 41-kilodalton glycoprotein isolated from the canine hookworm (Ancylostoma caninum) that potently inhibits CD11/CD18-dependent neutrophil function in vitro. NIF blocks the adhesion of activated human neutrophils to vascular endothelial cells as well as the release of H₂O₂ from activated neutrophils, over a similar concentration range. A cDNA encoding NIF was isolated from a canine hookworm cDNA library. NIF comprises a mature polypeptide of 257 amino acids, preceded by a 17-amino acid leader. The mature protein has 10 cysteines and has seven potential N-linked glycosylation sites. NIF is considered a prototype of a novel class of leukocyte function inhibitors.

Further characterization of the interaction of NIF with its integrin receptor showed that the A-domain of CR3 (CD11 b/CD18) is the specific binding site. Rieu, P. et al., J. Cell Biol. 127.2081-2091 (1994). The A-domain is a approximately 200-amino acid peptide present within structurally diverse proadhesive proteins including seven integrins. A recombinant form of the A-domain of beta 2 integrins CR3 and LFA-1 has been recently shown to bind divalent cations and to contain binding sites for protein ligands that play essential roles in leukocyte trafficking to inflammatory sites, phagocytosis and target cell killing. NIF was shown to be a selective CD11b A-domain binding protein. Thus, NIF bound directly, specifically and with high affinity (K_{d} of approximately 1 nM) to recombinant CD11b A-domain (r11bA). The NIF binding site in r11bA was mapped to four short peptides, one of which is an iC3b binding site. The interaction of NIF with CR3 in intact cells followed similar binding kinetics to those with r11bA, and occurred with similar affinity in resting and activated human neutrophils, suggesting that the NIF epitope is activation independent. Binding of NIF to CR3 blocked its ability to bind to its ligands (iC3b, fibrinogen, and CD54), and inhibited the ability of human neutrophils to ingest serum opsonized particles. NIF thus represents the first example of a “disintegrin” that targets the integrin A-domain, and is likely to be used by the hookworm to evade the host's inflammatory response. The unique structure of NIF, which lacks a “disintegrin motif” found in other known integrin blocking proteins, emphasizes basic structural differences in antagonists targeting A+ and A− integrins. Therefore, NIF is expected to be valuable in drug design efforts aimed at generating novel therapeutics. Rieu, P. et al., supra.

NIF has been found to exhibit a variety of beneficial effects in various inflammatory conditions. For instance, NIF has been found to be neuroprotective in a model of focal cerebral ischemia in the rat. Jiang, N., et al., Ann. Neurol 38.935-942 (1995). Thus, treatment with recombinant NIF resulted in a 48% reduction in cerebral infarction compared with control animals (p<0.01). The neuroprotective effect was correlated with a reduced number of neutrophils within the ischemic tissue. These results demonstrate potential therapeutic properties of rNIF in the management of stroke.

NIF also prevents neutrophil-dependent lung vascular injury in a guinea pig model. Barnard, J. W., et al, J. Immunol 155.4876-4881 (1995). Pulmonary vascular endothelial CD54 (ICAM-1) was induced in buffer-perfused lungs by exposure to TNF-α, and human neutrophils were added to the perfusate and activated by PMA. Lung injury (edema), as assessed by wet:dry weight ratio, and neutrophil uptake by lung myeloperoxidase (MPO) activity, were concomitantly inhibited by NIF. Endothelial monolayer experiments confirmed that NIF reduced neutrophil adherence. These studies indicated that NIF prevents neutrophil-dependent lung vascular injury by inhibiting neutrophil adhesion to the TNF-α-activated endothelium.

NIF also exhibits attenuation of the inflammatory response in an animal colitis. Meenan, J., et al., Scand. J. Gastroenterol. 31:786-791 (1996). Neutrophils are significant effector cells in acute inflammatory bowel disease. Recruitment of these cells is dependent on beta 2-integrin-mediated adhesion and transmigration. The efficacy of NIF, as an antagonist of the beta 2-integrin CD11b/CD18, in ameliorating inflammation was tested in an animal model of acute colitis. Immune-complex colitis was induced in groups of rabbits by using various formalin concentrations. Animals were treated with rNIF, 10 mg/kg. Mucosal appearance was scored, and tissue was saved for histology and quantitation of several markers of inflammation. NIF generally reduced these level of the inflammation markers, and histology showed polymorphonuclear cell infiltration to be reduced by rNIF, suggesting that blockade of CD11b/CD18-mediated mucosal neutrophil recruitment may form part of a strategy for targeted therapeutic intervention in inflammatory bowel disease.

In addition, NIF reduces leukocyte adhesion in the liver after hemorrhagic shock. Bauer, C., et al., Shock 4:187-192 (1995). This study was designed to assess the effect of NIF on hepatic leukocyte trafficking by intravital microscopy 5 h after hemorrhagic shock. Anesthetized rats were instrumented for invasive hemodynamical monitoring. Hemorrhagic shock was induced for 60 min by withdrawal of arterial blood (mean arterial blood pressure=40 mm Hg). Rats were adequately resuscitated for 5 h to achieve a mean arterial blood pressure>100 mm Hg and were randomly assigned to blinded treatment with NIF or placebo control protein administered as a single intravenous bolus (10 mg/kg) at the time of resuscitation. Intrahepatic leukocyte adhesion was evaluated by in vivo fluorescence microscopy. There were no significant differences observed in hemodynamic parameters between the shock groups throughout the study. However, NIF significantly reduced firm leukocyte adhesion in liver sinusoids, indicating that NIF may be beneficial in the attenuation of the pathological shock-induced leukocyte adhesion.

Recently, the cloning of a human cDNA encoding a protein called GliPR (glioma pathogenesis-related protein) has been reported. Murphy, E. V., el al, Gene 159:131-135 (1995). This protein is structurally similar to plant pathogenesis-related proteins and is expressed specifically in brain tumors. More particularly, the GliPR gene is. highly expressed in the human brain tumor, glioblastoma multiforme/astrocytoma, but neither in normal fetal or adult brain tissue, nor in other nervous system tumors. GliPR shares up to 50% amino acid homology with plant pathogenesis-related proteins, group 1, over a region that comprises almost two thirds of the protein.

Nevertheless, there is a continuing need to identify human polypeptides which are effectors of defense functions including antipathogen functions as well as immune system functions related to inflammation, particularly functions of polymorphonuclear leukocytes (neutrophils), for instance, for development of new antimicrobial and anti-inflammatory agents.

### SUMMARY OF THE INVENTION

In one aspect, the present invention provides an isolated nucleic acid molecule comprising a polynucleotide encoding at least a portion of the Leukocyte Regulatory Factor-1 (LRF-1) polypeptide having the complete amino acid sequence shown in SEQ ID NO:2 or the complete amino acid sequence encoded by the cDNA clone deposited in plasmid DNA at the American Type Culture Collection (ATCC), 10801 University Boulevard, Manassas, Va. 20110-2209, and given the ATCC Deposit Number 97860 on Jan. 29, 1997. The nucleotide sequence determined by sequencing the deposited LRF-1 clone, which is shown in FIGS. 1A and 1B (SEQ ID NO:1), contains an open reading frame encoding a complete polypeptide of 279 amino acid residues, including an initiation codon encoding an N-terminal methionine at nucleotide positions 31-33, and a predicted molecular weight of about 32 kDa. Nucleic acid molecules of the invention include those encoding the complete amino acid sequence (optionally excepting the N-terminal methionine) shown in SEQ ID NO:2, or the complete amino acid sequence (optionally excepting the N-terminal methionine) encoded by the cDNA clone in ATCC Deposit Number 97860. Nucleic acid molecules comprising an amino acid sequence above, advantageously those not encoding the N-terminal methionine, also may encode additional amino acids fused to the N-terminus of the LRF-1 amino acid sequence.

In another aspect, the present invention also provides isolated nucleic acid molecules comprising a polynucleotide encoding at least a portion of the LRF-2 polypeptide having the complete amino acid sequence shown in SEQ ID NO:4 or the complete amino acid sequence encoded by the cDNA clone deposited as plasmid DNA under ATCC Deposit Number 97867 on Feb. 6, 1997. The nucleotide sequence determined by sequencing the deposited LRF-2 clone, which is shown in FIGS. 2A, 2B, 2C, and 2D (SEQ ID NO:3), contains an open reading frame encoding a complete polypeptide of 463 amino acid residues, including an initiation codon encoding an N-terminal methionine at nucleotide positions 211-213, and a predicted molecular weight of about 50 kDa. Nucleic acid molecules of the invention include those encoding the complete amino acid sequence (optionally excepting the N-terminal methionine) shown in SEQ ID NO:4, or the complete amino acid sequence (optionally excepting the N-terminal methionine) encoded by the cDNA clone in ATCC Deposit Number 97867. Such molecules also may encode additional amino acids fused to the N-terminus of the LRF-2 amino acid sequence.

The LRF-1 and LRF-2 proteins of this invention includes several amino acid sequence motifs which are characteristic of certain known protein families. Thus, LRF-1 and LRF-2 share extensive amino acid sequence homology with the protein called “Neutrophil Inhibitory Factor (NIF),” the 41-kilodalton glycoprotein isolated from the canine hookworm (Ancylostoma caninum) that potently inhibits CD 11/CD 18-dependent neutrophil function in vitro and, therefore, is considered a prototype of a novel class of leukocyte function inhibitors. Moyle, M., et al., supra. The complete NIF polypeptide includes a sequence of 274 amino acids (SEQ ID NO:5) which comprises a mature polypeptide of 257 amino acids, preceded by a 17-amino acid leader. The mature form (secreted portion) of the protein has 10 cysteines, several of which several are conserved in both LRF-1 and LRF-2. See, FIGS. 3, 4, and 5.

The LRF-1 and LRF-2 proteins of the present invention also share sequence homology with the translation product of a human mRNA for the protein known as GliPR (glioma pathogenesis-related protein; SEQ ID NO:6) described above. Murphy, E. V., et al., supra. See, FIGS. 3, 4, and 5. This homology includes much of the C-terminal cysteine-rich domain found so far in all members of the human cysteine-rich secretory protein (CRISP) family typified by human TPX-1. Kratzschmar, J. et al, supra.

In addition, the LRF-2 amino acid sequence contains two signature sequences which are located in the C-terminal half of many CRISP protein family members: 1) the sequence GHYTQVVWAKT (SEQ ID NO:21) at positions 127 to 137 in FIGS. 2A, 2B, 2C, and 2D (positions 105 to 115 of SEQ ID NO:4) and 2) the sequence LLVCNYEPPGNV (SEQ ID NO:22) at positions 160 to 171 in FIGS. 2A, 2B, 2C, and 2D (positions 138 to 149 of SEQ ID NO:4). These signature sequences are also highly, although not identically, conserved in the C-terminal region of the LRF-1 amino acid sequence (at about positions 139 to 149 and about 170 to 181 in FIGS. 1A and 1B (respectively, positions 114 to 124 and 145 to 156 of SEQ ID NO:2).

The homology shared with the canine hookworm NIF polypeptide, as well as with the related plant pathogenesis-related (PR) proteins, indicates that the human LRF-1 and LRF-2 polypeptides also exhibit activities useful for modulation of immune system cell functions such as proliferation, differentiation, migration, adhesion and activation of leukocytes, particularly neutrophils, which ultimately permits modulation of defensive functions of these cells such as antimicrobial and anti-inflammatory activities.

The complete LRF-2 amino acid sequence (SEQ ID NO:4) also contains a peroxidase “signature” sequence (i.e., the amino acid sequence EVPSILAAHSL (SEQ ID NO:23) at positions 287-297 of FIGS. 2A, 2B, 2C, and 2D (positions 265-275 of SEQ ID NO:4). Peroxidases (EC 1.11.1.-) are heme-binding enzymes that carry out a variety of biosynthetic and degradative functions using hydrogen peroxide as the electron acceptor. Peroxidases are widely distributed throughout bacteria, fungi, plants, and vertebrates, including, for instance, the following: myeloperoxidase (EC 1.11.1.7) (MPO), which is found in granulocytes and monocytes and plays a major role in the oxygen-dependent microbicidal system of neutrophils; lactoperoxidase (EC 1.11.1.7) (LPO), which is a milk protein that acts as an antimicrobial agent; eosinophil peroxidase (EC 1.11.1.7) (EPO), an enzyme found in the cytoplasmic granules of eosinophils; and plant peroxidases (EC 1.11.1.7), some of which are expressed as a defense response toward wounding while others are involved in the metabolism of auxin and the biosynthesis of lignin. Since a major function of neutrophils involves release of toxic hydrogen peroxide, the peroxidase “signature” sequence in LRF-2 indicates that this particular protein is involved in carrying out biosynthetic and/or degradative functions (e.g., inflammatory and/or antimicrobial activities) using hydrogen peroxide released from neutrophils as the electron acceptor. In contrast the amino acid sequence of LRF-1 (FIGS. 1A and 1B and SEQ ID NO:2), while highly homologous with that of LRF-2 over the N-terminal region, terminates prior to the C-terminal region of LRF-2 containing the peroxidase signature sequence (See, FIGS. 3, 4, and 5).

The encoded LRF-1 polypeptide has a predicted secretory leader (signal peptide) sequence of about 25 amino acids underlined in FIGS. 1A and 1B; and the amino acid sequence of the predicted mature LRF-1 protein is also shown, as amino acid residues 26-279 in FIGS. 1A and 1B (residues 1-254 in SEQ ID NO:2). For the encoded LRF-2 polypeptide, two leader sequences are predicted, one of about 20 amino acids (broken underline in FIGS. 2A, 2B, 2C, and 2D) and the other of about 22 amino acids (solid underline in FIGS. 2A, 2B, 2C, and 2D); and the amino acid sequence of the respectively predicted mature forms of the LRF-2 protein are also shown, as amino acid residues 21-463 or 23-463, respectively in FIGS. 2A, 2B, 2C, and 2D (residues −2 to 441 or +1 to 441, respectively, in SEQ ID NO:4). In addition, the encoded LRF-2 amino acid sequence shown in FIGS. 2A, 2B, 2C, and 2D includes a hydrophobic C-terminal sequence comprising a predicted transmembrane domain of about 16 amino acids (i.e., the sequence PGHVMGPLLGLLLLPP (SEQ ID NO:24) underlined in FIGS. 2A, 2B, 2C, and 2D) comprising amino acid number about 441 to about 456 in FIGS. 2A, 2B, 2C, and 2D (positions 419 to 434 in SEQ ID NO:4), indicating that at least one form of LRF-2 can be membrane bound (that is, a type I integral membrane protein, anchored by a single transmembrane domain at the C-terminus). Accordingly, the invention also provides a nucleic acid molecule encoding a soluble form of a mature LRF2 protein lacking about the 23 amino acids at the C-terminus comprising the predicted C-terminal transmembrane domain, which comprises “extracellular domain” of the amino acid sequence, comprising residues from about 21 to about 440 or from about 23 to about 440 in FIGS. 2A, 2B, 2C, and 2D (residues −2 to 418 or +1 to 418, respectively, in SEQ ID NO:4). Such a soluble form is particularly preferred for applications such as therapeutic uses where the protein is to be used (e.g., administered to a patient) in a liquid formulation.

The invention also provides variant cDNA forms of LRF-2 mRNA. Thus, one cDNA clone has been found (in a library made from human amygdala tissue) which lacks two portions of the nucleotide sequence shown in FIGS. 2A, 2B, 2C, and 2D, namely nucleotides 700 to 1279 and nucleotides 1420 to 1842, which are underlined in FIGS. 2A, 2B, 2C, and 2D (and are numbered identically in SEQ ID NO:3). This clone therefore appears to represent a splicing variant of the LRF-2 mRNA which comprises the complete sequence shown in FIGS. 2A, 2B, 2C, and 2D. Further, sequencing of four independent cDNA clones indicates that the usually spliced (“mature”) form of LRF-2 mRNA ends at nucleotide 2288 in FIGS. 2A, 2B, 2C, and 2D (and SEQ ID NO:3), indicating that the approximately 1.1 kb of sequence 3′ of nucleotide 2288 in FIGS. 2A, 2B, 2C, and 2D most likely is due to incomplete splicing of the mRNA encoded by this particular cDNA. Northern blot analyses of tissues expressing LRF-2 mRNA have so far shown only a single mRNA species of about 2.4 kb, further indicating that about 1.1 kb of sequence at the 3′ end of the nucleotide sequence in FIGS. 2A, 2B, 2C, and 2D (SEQ ID NO:3) is not included in the most common form of LRF-2 mRNA.

More in particular, therefore, one aspect of the invention provides an isolated nucleic acid molecule comprising a polynucleotide comprising a nucleotide sequence selected from the group consisting of: (a) a nucleotide sequence encoding the LRF-1 polypeptide having the complete amino acid sequence in SEQ ID NO:2 excepting the N-terminal methionine (i.e., positions −24 to +254 of SEQ ID NO:2); (b) a nucleotide sequence encoding the predicted mature LRF-1 polypeptide having the amino acid sequence at positions 1-254 in SEQ ID NO:2; (c) a nucleotide sequence encoding the LRF-1 polypeptide having the complete amino acid sequence excepting the N-terminal methionine encoded by the cDNA clone contained in ATCC Deposit No 97860; (d) a nucleotide sequence encoding the mature LRF-1 polypeptide having the amino acid sequence encoded by the cDNA clone contained in ATCC Deposit No. 97860; and (e) a nucleotide sequence complementary to any of the nucleotide sequences in (a), (b), (c) or (d) above.

Another aspect of the invention provides an isolated nucleic acid molecule comprising a polynucleotide comprising a nucleotide sequence selected from the group consisting of: (a) a nucleotide sequence encoding the LRF-2 polypeptide having the complete amino acid sequence in SEQ ID NO:4 excepting the N-terminal methionine (i.e., positions −21 to +441 of SEQ ID NO:4); (b) a nucleotide sequence encoding the predicted mature LRF-2 polypeptide having the amino acid sequence at positions −2 to +441 or at positions +1 to +441 in SEQ ID NO:4; (c) a nucleotide sequence encoding the predicted soluble mature (extracellular domain of the) LRF-2 polypeptide having the amino acid sequence at about position −2 to about position 418 or at about position +1 to about position 418 in SEQ ID NO:4; (d) a nucleotide sequence encoding the LRF-2 polypeptide having the complete amino acid sequence excepting the N-terminal methionine encoded by the cDNA clone contained in ATCC Deposit No 97867; (e) a nucleotide sequence encoding the mature LRF-2 polypeptide having the amino acid sequence encoded by the cDNA clone contained in ATCC Deposit No. 97867; (f) a nucleotide sequence encoding the LRF-2 polypeptide having the amino acid sequence of the mature LRF-2 polypeptide encoded by the cDNA clone contained in ATCC Deposit No. 97867 excepting the C-terminal sequence of about 23 amino acids of the mature LRF-2 polypeptide encoded by that cDNA; and (g) a nucleotide sequence complementary to any of the nucleotide sequences in (a), (b), (c), (d), (e) or (f) above.

Further embodiments of the invention include isolated nucleic acid molecules that comprise a polynucleotide having a nucleotide sequence at least 90% identical, and more preferably at least 95%, 96%, 97%, 98% or 99% identical, to any of the LRF-1 nucleotide sequences in (a), (b), (c) or (d), above, or to any of the LRF-2 sequences in (a), (b), (c), (d), (e) or (f), above, or a polynucleotide which hybridizes under stringent hybridization conditions to an LRF-1 or to an LRF-2 polynucleotide, above. This polynucleotide which hybridizes does not hybridize under stringent hybridization conditions to a polynucleotide having a nucleotide sequence consisting of only A residues or of only T residues. An additional nucleic acid embodiment of the invention relates to an isolated nucleic acid molecule comprising a polynucleotide which encodes the amino acid sequence of an epitope-bearing portion of a LRF-1 polypeptide or an LRF-2 polypeptide having an amino acid sequence described above.

The present invention also relates to recombinant vectors, which include the isolated nucleic acid molecules of the present invention, and to host cells containing the recombinant vectors, as well as to methods of making such vectors and host cells and for using them for production of LRF-1 polypeptides or LRF-2 polypeptides by recombinant techniques.

The invention further provides an isolated LRF-1 polypeptide comprising an amino acid sequence selected from the group consisting of: (a) the complete amino acid sequence of the full-length LRF-1 polypeptide sequence shown in SEQ ID NO:2 excepting the N-terminal methionine (i.e., positions −24 to +254 of SEQ ID NO:2); (b) the amino acid sequence of the predicted mature LRF-1 polypeptide shown at positions +1 to +254 in SEQ ID NO:2; (c) the complete amino acid sequence of the LRF-1 excepting the N-terminal methionine encoded by the cDNA clone contained in ATCC Deposit No 97860; and (d) the amino acid sequence of the mature LRF-1 polypeptide encoded by the cDNA clone contained in ATCC Deposit No. 97860.

Also provided is an isolated LRF-2 polypeptide comprising an amino acid sequence selected from the group consisting of: (a) the complete amino acid sequence of the full-length LRF-2 polypeptide shown in SEQ ID NO:4 excepting the N-terminal methionine (i.e., positions −21 to +441 of SEQ ID NO:4); (b) the amino acid sequence of the predicted mature LRF-2 polypeptide shown at about position −2 to about position +441 or at about position +1 to about position +441 in SEQ ID NO:4; (c) the amino acid sequence of the predicted soluble mature LRF-2 shown at about position −2 to about position +418 or at about position +1 to about position +418 in SEQ ID NO:4; (d) the complete amino acid sequence of the full-length LRF-2 polypeptide excepting the N-terminal methionine encoded by the cDNA clone contained in ATCC Deposit No 97867; (e) the amino acid sequence of the mature LRF-2 polypeptide encoded by the cDNA clone contained in ATCC Deposit No. 97867; and (f) the amino acid sequence of the soluble mature LRF-2 polypeptide encoded by the cDNA clone contained in ATCC Deposit No. 97867 where the soluble form lacks the C-terminal sequence of about 23 amino acids of the mature LRF-2 polypeptide encoded by that cDNA.

The polypeptides of the present invention also include polypeptides having an amino acid sequence at least 80% identical, more preferably at least 90% identical, and still more preferably 95%, 96%, 97%, 98% or 99% identical to those described for LRF-1 in (a), (b), (c)or (d) above, or for LRF-2, in (a), (b), (c), (d), (e) or (f) above, as well as polypeptides having an amino acid sequence with at least 90% similarity, and more preferably at least 95% similarity, to those above.

An additional embodiment of this aspect of the invention relates to a peptide or polypeptide which comprises the amino acid sequence of an epitope-bearing portion of an LRF-1 or LRF-2 polypeptide having an amino acid sequence described above. Peptides or polypeptides having the amino acid sequence of an epitope-bearing portion of a LRF-1 or LRF-2 polypeptide of the invention include portions of such polypeptides with at least six or seven, preferably at least nine, and more preferably at least about 30 amino acids to about 50 amino acids, although epitope-bearing polypeptides of any length up to and including the entire amino acid sequence of a polypeptide of the invention described above also are included in the invention.

In another embodiment, the invention provides an isolated antibody that binds specifically to an LRF-1 or to an LRF-2 polypeptide having an amino acid sequence described above. The invention further provides methods for isolating antibodies that bind specifically to an LRF-1 or LRF-2 polypeptide having an amino acid sequence as described herein. Such antibodies are useful diagnostically or therapeutically as described below.

The invention also provides for pharmaceutical compositions comprising LRF-1 or LRF-2 polypeptides, particularly human LRF-1 or LRF-2 polypeptides, which may be employed, for instance, to treat immune system disorders. Methods of treating individuals in need of LRF-1 or LRF-2 polypeptides are also provided.

The invention further provides compositions comprising a LRF-1 or an LRF-2 polynucleotide or an LRF-1 LRF-2 polypeptide, for administration to cells in vitro, to cells ex vivo and to cells in vivo, or to a multicellular organism. In certain particularly preferred embodiments of this aspect of the invention, the compositions comprise an LRF-1 or LRF-2 polynucleotide for expression, respectively, of an LRF-1 or LRF-2 polypeptide in a host organism for treatment of disease. Particularly preferred in this regard is expression in a human patient for treatment of a dysfunction associated with aberrant endogenous activity of an LRF-1 or LRF-2 gene.

The present invention also provides a screening method for identifying compounds capable of enhancing or inhibiting a biological activity of the LRF-1 or LRF-2 polypeptide of the invention, which involves contacting a cell bearing receptors which specifically bind an LRF-1 or LRF-2 polypeptide and having a function which is modulated by such binding, with a candidate compound in the presence, respectively, of an LRF-1 or LRF-2 polypeptide, assaying a function of the receptor-bearing cell in the presence of the candidate compound and, respectively, of the LRF-1 or LRF-2 polypeptide, and comparing the level of that cellular function to a standard level of such activity, the standard being assayed when contact is made between the receptor-bearing cell in the presence of the LRF-1 or LRF-2 polypeptide, respectively, and the absence of the candidate compound In this assay, an increase in that cellular function over the standard indicates that the candidate compound is an agonist of LRF-1 or LRF-1 activity and a decrease in that function compared to the standard indicates that the compound is an antagonist of LRF-1 or LRF-2 activity. In one embodiment of this aspect of the invention, the screening assay for agonists and antagonists involves determining the effect a candidate compound has on LRF-1 or LRF-2 polypeptide binding to a receptor which specifically binds that polypeptide. In particular, the method involves contacting the receptor with an LRF-1 or LRF-2 polypeptide and a candidate compound and determining whether binding of that polypeptide to the receptor is increased or decreased due to the presence of the candidate compound. In this assay, an increase in binding of LRF-1 or LRF-2 polypeptide over the standard binding indicates that the candidate compound is an agonist of LRF-1 or LRF-2 binding activity and a decrease in LRF-1 or LRF-2 binding compared to the standard indicates that the compound is an antagonist of LRF-1 or LRF-2 binding activity.

It has been discovered that LRF-1 is expressed not only in human testes but also in dendritic cells (DC) which are the principal antigen presenting cells involved in primary immune responses; their major function is to obtain antigen in tissues, migrate to lymphoid organs, and activate T cells (Mohamadzadeh, M. et al., J. Immunol. 156: 3102-3106 (1996).

It has further been discovered that a nucleotide sequence encoding the mature LRF-2 polypeptide having the amino acid sequence encoded by the cDNA clone contained in the host identified as ATCC Deposit No. 97867 is detectable by Northern blot not only in human fetal heart tissue where the deposited clone originated, but also in skeletal muscle and pancreas at much lower levels. Individual cDNA clones encoding all or part of the LRF-2 amino acid (SEQ ID NO:4) also have been isolated from amygdala, fetal epithelium, striatum, microvascular endothelium, Jurkat T cells, breast, rhabdomyosarcoma, fetal bone, and smooth muscle.

Therefore, nucleic acids of the invention are useful in the first instance (alone or in combination with other nucleic acids) as hybridization probes for differential identification of the tissue(s) or cell type(s) present in a biological sample. Similarly, polypeptides and antibodies directed to those polypeptides are useful to provide immunological probes for differential identification of the tissue(s) or cell type(s). In addition, for a number of disorders of the above tissues or cell s, significantly higher or lower levels of LRF-1 or LRF-2 gene expression may be detected in certain tissues (e.g., cancerous and wounded tissues) or bodily fluids (e.g., serum, plasma, urine, synovial fluid or spinal fluid) taken from an individual having such a disorder, relative to a “standard” LRF-1 or LRF-2 gene expression level, i.e., the expression level in healthy tissue from an individual not having the immune system disorder. Thus, the invention provides a diagnostic method useful during diagnosis of such a disorder, which involves: (a) assaying LRF-1 or LRF-2 gene expression level in cells or body fluid of an individual; (b) comparing the LRF-1 or LRF-2 gene expression level, respectively, with a standard LRF-1 or LRF-2 gene expression level, whereby an increase or decrease in the assayed gene expression level compared to the standard expression level is indicative of disorder in the pertinent system.

An additional aspect of the invention is related to a method for treating an individual in need of an increased level of LRF-1 or of LRF-2 activity in the body comprising administering to such an individual a composition comprising a therapeutically effective amount, respectively, of an isolated LRF-1 or LRF-2 polypeptide of the invention or an agonist thereof. A still further aspect of the invention is related to a method for treating an individual in need of a decreased level of LRF-1 or of LRF-2 activity in the body comprising, administering to such an individual a composition comprising a therapeutically effective amount, respectively, of an LRF-1 or an LRF-2 antagonist. Preferred antagonists for use in the present invention are LRF-1-specific antibodies or LRF-2-specific antibodies.

### BRIEF DESCRIPTION OF THE FIGURES

FIGS. 1A and 1B show the nucleotide sequence (SEQ ID NO:1) and deduced amino acid sequence (SEQ ID NO:2) of LRF-1. The predicated secretory leader sequence of 25 amino acids at the amino terminus is underlined. FIGS. 2A, 2B, 2C, and 2D show the nucleotide sequence (SEQ ID NO:3) and deduced amino acid sequence (SEQ ID NO:4) of LRF-2. Two predicted leader sequences are shown at the amino terminus, the first consisting of the first 20 amino acids (broken underline) and the second consisting of the first 22 amino acids (solid underline). Also shown is a hydrophobic C-terminal amino acid sequence comprising a predicted transmembrane domain of about 16 amino acids at positions 441 to 456 (solid underline) (positions 419 to 434 in SEQ ID NO:4). Two portions of the nucleotide sequence missing in one cDNA clone found (in a library made from human amygdala tissue also are indicated (solid underline) at nucleotides 700 to 1279 and nucleotides 1420 to 1842 (numbered identically in SEQ ID NO:3). Note that the methionine residue at the beginning of each leader sequence in FIGS. 1A and 1B and FIGS. 2A, 2B, 2C, and 2D is shown in position number (positive) 1, whereas the leader positions in the corresponding sequences of SEQ ID NO:2 and SEQ ID NO:4 are designated with negative position numbers. For example, the leader sequence positions 1 to 25 in FIGS. 1A and 1B correspond to positions −25 to −1 in SEQ ID NO:2, while the leader sequence positions 1 to 20 in FIGS. 2A, 2B, 2C, and 2D correspond to positions −22 to −3 in SEQ ID NO:4.

FIG. 3 shows the regions of identity between the amino acid sequence of the LRF-1 protein and the amino acid sequence of the protein called “Neutrophil Inhibitory Factor (NIF)” (SEQ ID NO:5), the 41-kilodalton glycoprotein isolated from the canine hookworm (Ancylostoma caninum) that potently inhibits CD11/CD18-dependent neutrophil function in vitro (Moyle, M., et al., supra) determined by the computer program “Bestfit” (Wisconsin Sequence Analysis Package, Version 8 for Unix, Genetics Computer Group, University Research Park, 575 Science Drive, Madison, Wis. 53711) using the default parameters. FIG. 4 similarly shows the regions of identity between the amino acid sequence of the LRF-2 protein and that of NIF, determined by Bestfit as above. FIG. 5 shows a simultaneous comparison of the amino sequences of the NIF polypeptide (labeled with the GenBank Accession Number as “A54419 NIF” (SEQ ID NO:5)) with the following amino acid sequences (from top to bottom line): a human protein called GliPR (glioma pathogenesis-related protein; labeled with GenBank Number as “U16307” (SEQ ID NO:6) reported by Murphy, E. V., et al., supra); the LRF-1 protein (labeled “HTEIX55XXa protein” (SEQ ID NO:2) in which “HTEIX55” represents the laboratory identifier of the deposited clone); and the LRF-2 protein (labeled “HHFFQ13X protein” (SEQ ID NO:4) in which “HHFFQ13” represents the laboratory identifier of the deposited clone). This alignment was performed using the “Megalign” routine in the DNAStar program.

FIG. 6 shows an analysis of the LRF-1 amino acid sequence. Alpha, beta, turn and coil regions; hydrophilicity and hydrophobicity; amphipathic regions; flexible regions; antigenic index and surface probability are shown. In the “Antigenic Index—Jameson-Wolf” graph, the positive peaks indicate locations of the highly antigenic regions of the LRF-1 protein, i.e., regions from which epitope-bearing peptides of the invention can be obtained. FIG. 7 shows a comparable analysis of the LRF-2 amino acid sequence.

### DETAILED DESCRIPTION

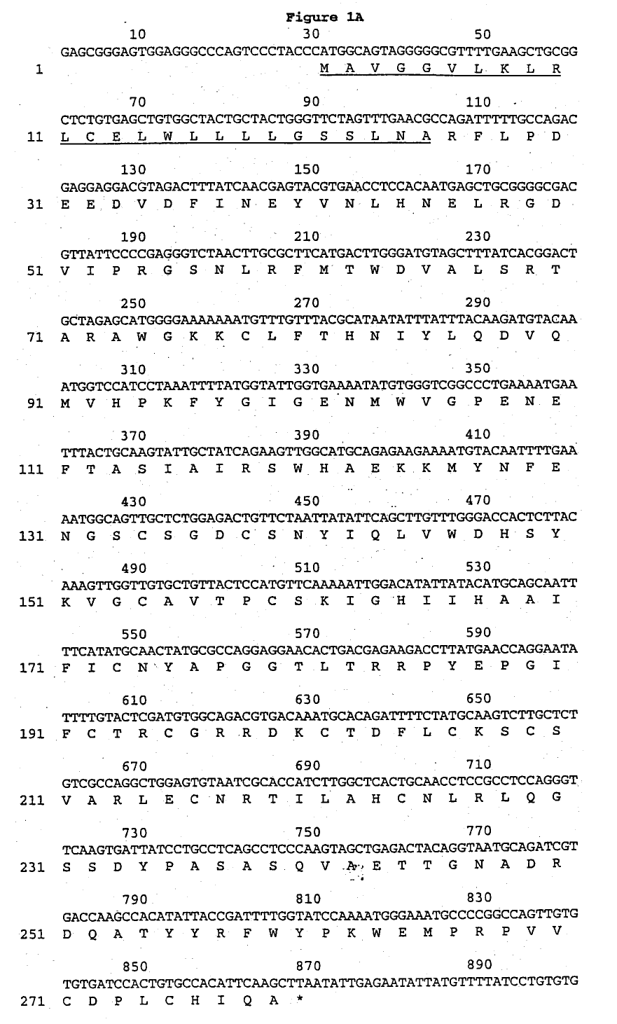

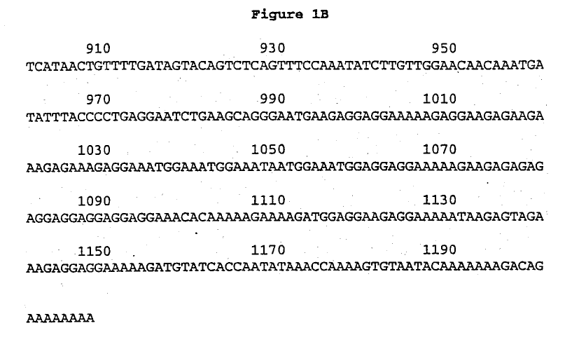

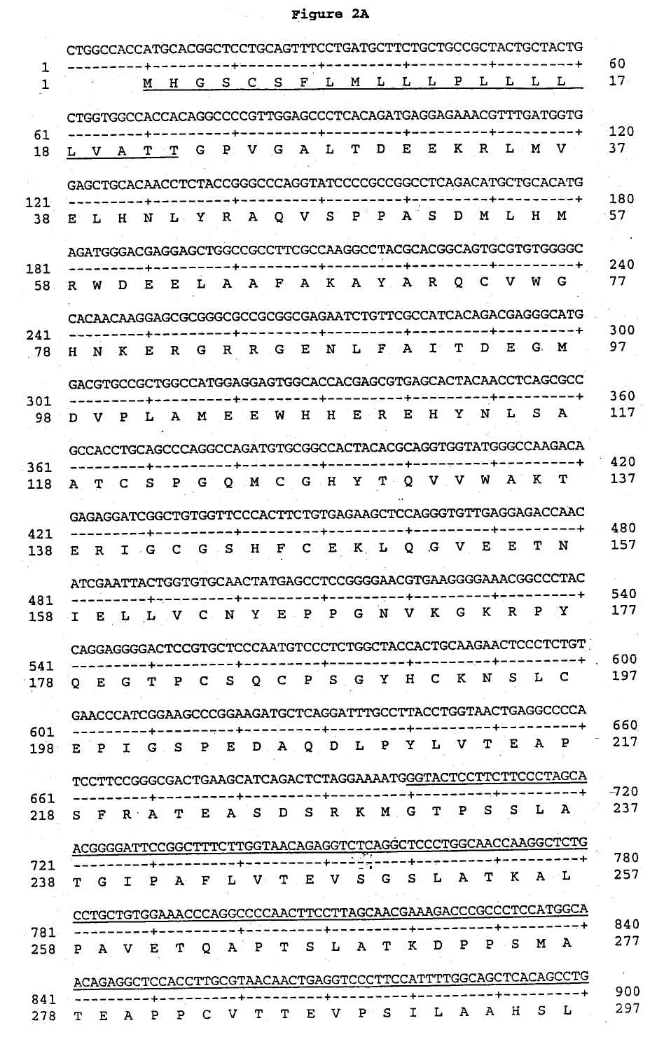

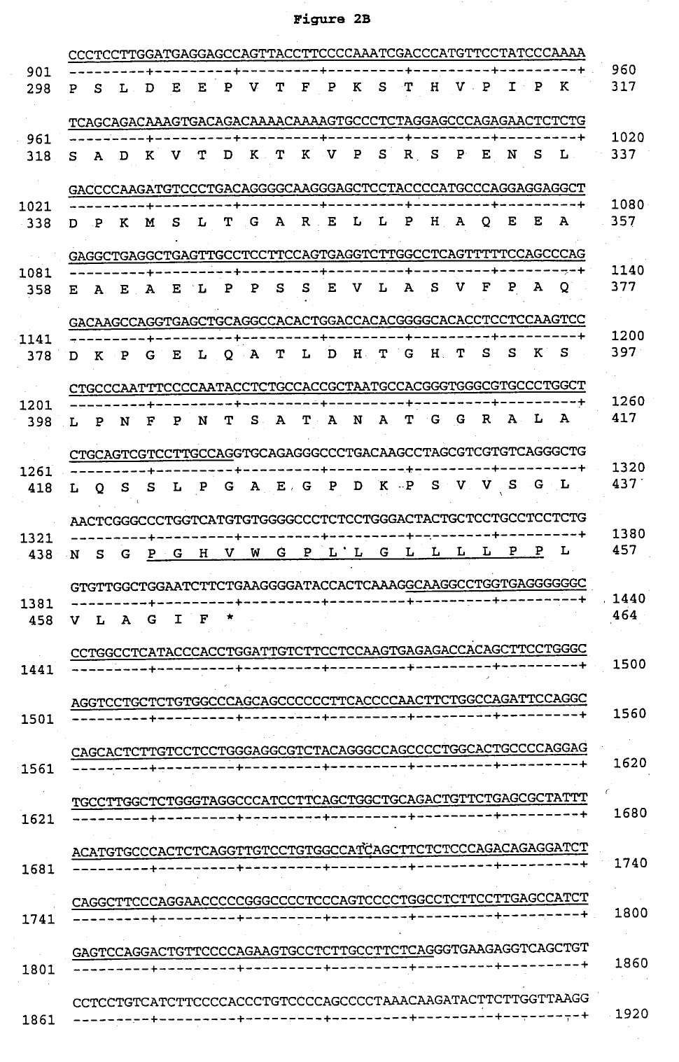

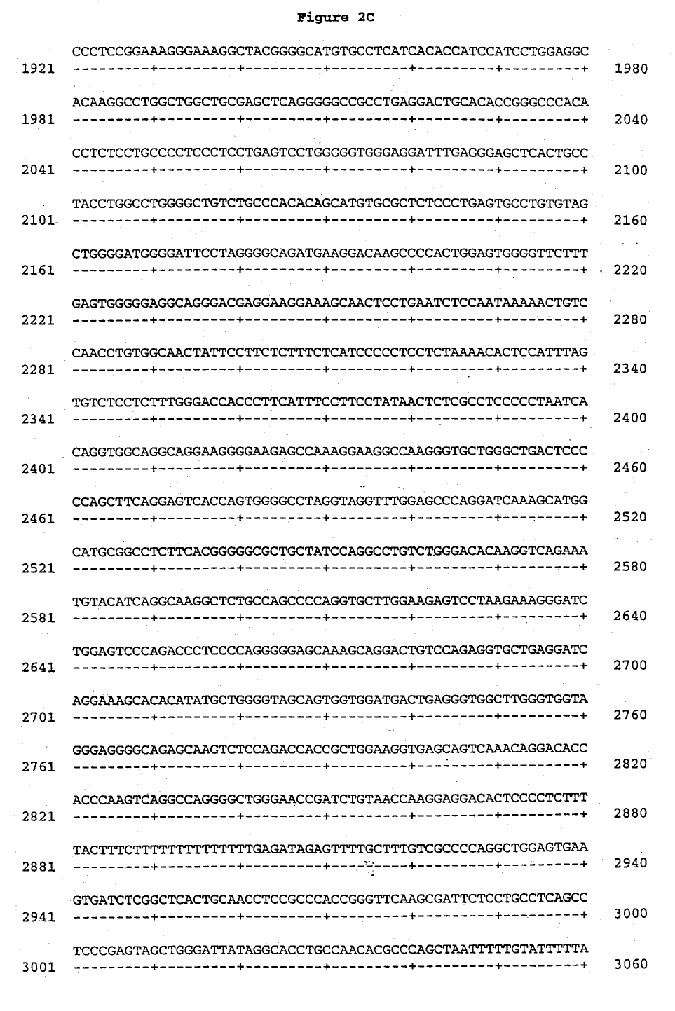

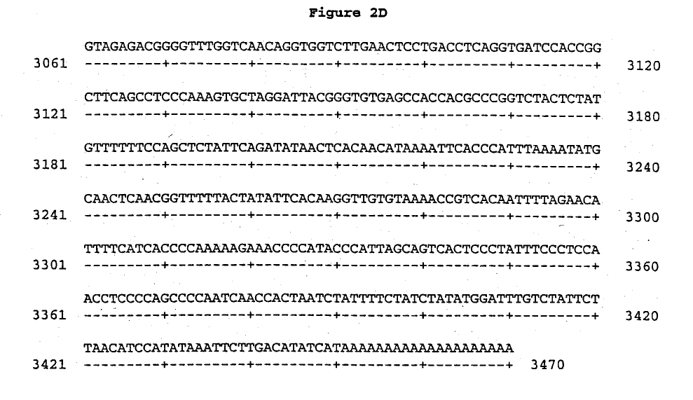

The present invention provides isolated nucleic acid molecules comprising a polynucleotide encoding an LRF-1 polypeptide having the amino acid sequence shown in SEQ ID NO:2, which was determined by sequencing a cloned cDNA. The nucleotide sequence shown in FIGS. 1A and 1B (SEQ ID NO: 1) was obtained by sequencing the HTEIX55 cDNA clone, which was deposited on Jan. 29, 1997 at the ATCC, and given accession number ATCC 97860. The deposited clone is contained in the pBluescript SK(−) plasmid (Stratagene, La Jolla, Calif.).

The invention also provides isolated nucleic acid molecules comprising a polynucleotide encoding an LRF-2 polypeptide having the amino acid sequence shown in SEQ ID NO:4, which was determined by sequencing a cloned cDNA. The nucleotide sequence shown in FIGS. 2A, 2B, 2C, and 2D (SEQ ID NO:3) was obtained by sequencing the HHFFQ13 cDNA clone, which was deposited on Feb. 6, 1997 at the ATCC, and given accession number ATCC 97867. This deposited clone also is contained in the pBluescript SK(−) plasmid (Stratagene, La Jolla, Calif.).

### Nucleic Acid Molecules

Unless otherwise indicated, all nucleotide sequences determined by sequencing a DNA molecule herein were determined using an automated DNA sequencer (such as the Model 373 from Applied Biosystems, Inc., Foster City, Calif.), and all amino acid sequences of polypeptides encoded by DNA molecules determined herein were predicted by translation of a DNA sequence determined as above. Therefore, as is known in the art for any DNA sequence determined by this automated approach, any nucleotide sequence determined herein may contain some errors. Nucleotide sequences determined by automation are typically at least about 90% identical, more typically at least about 95% to at least about 99.9% identical to the actual nucleotide sequence of the sequenced DNA molecule. The actual sequence can be more precisely determined by other approaches including manual DNA sequencing methods well known in the art. As is also known in the art, a single insertion or deletion in a determined nucleotide sequence compared to the actual sequence will cause a frame shift in translation of the nucleotide sequence such that the predicted amino acid sequence encoded by a determined nucleotide sequence will be completely different from the amino acid sequence actually encoded by the sequenced DNA molecule, beginning at the point of such an insertion or deletion.

By “nucleotide sequence” of a nucleic acid molecule or polynucleotide is intended, for a DNA molecule or polynucleotide, a sequence of deoxyribonucleotides, and for an RNA molecule or polynucleotide, the corresponding sequence of ribonucleotides (A, G, C and U), where each thymidine deoxyribonucleotide (T) in the specified deoxyribonucleotide sequence is replaced by the ribonucleotide uridine (U).

Using the information provided herein, such as the nucleotide sequence in FIGS. 1A and 1B and FIGS. 2A, 2B, 2C, and 2D (SEQ ID NO:1 or SEQ ID NO:3), a nucleic acid molecule of the present invention encoding an LRF-1 or LRF-2 polypeptide may be obtained using standard cloning and screening procedures, such as those for cloning cDNAs using mRNA as starting material. Illustrative of the invention, the LRF-1 nucleic acid molecule described in FIGS. 1A and 1B (SEQ ID NO:1) was discovered in a cDNA library derived from human testes. Additional clones of the same gene were also identified in a cDNA libraries from human dendritic cells.

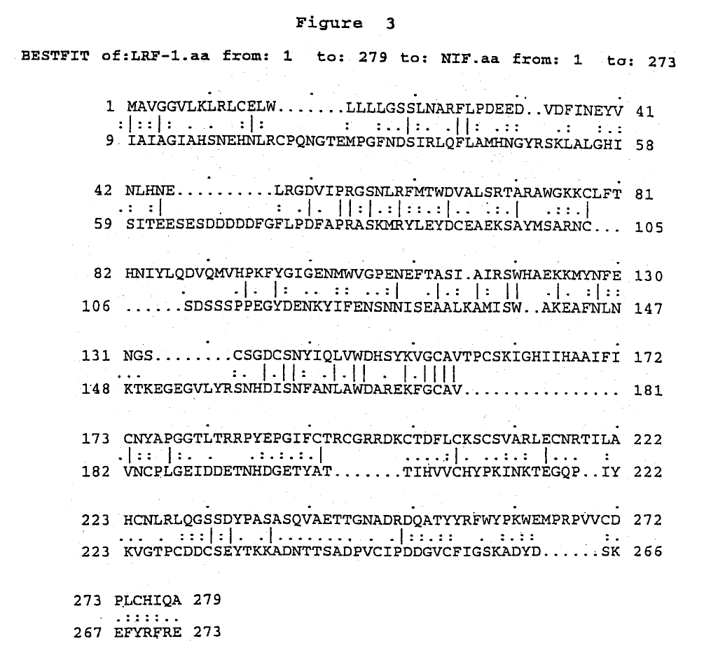

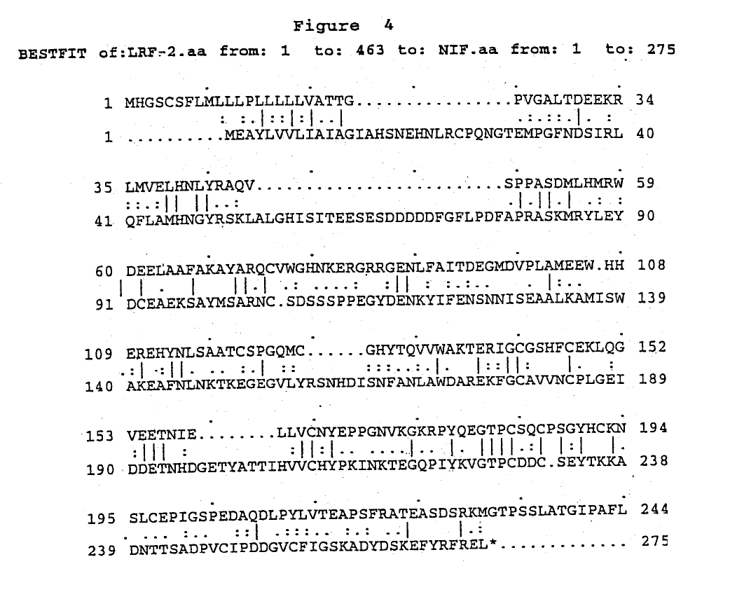

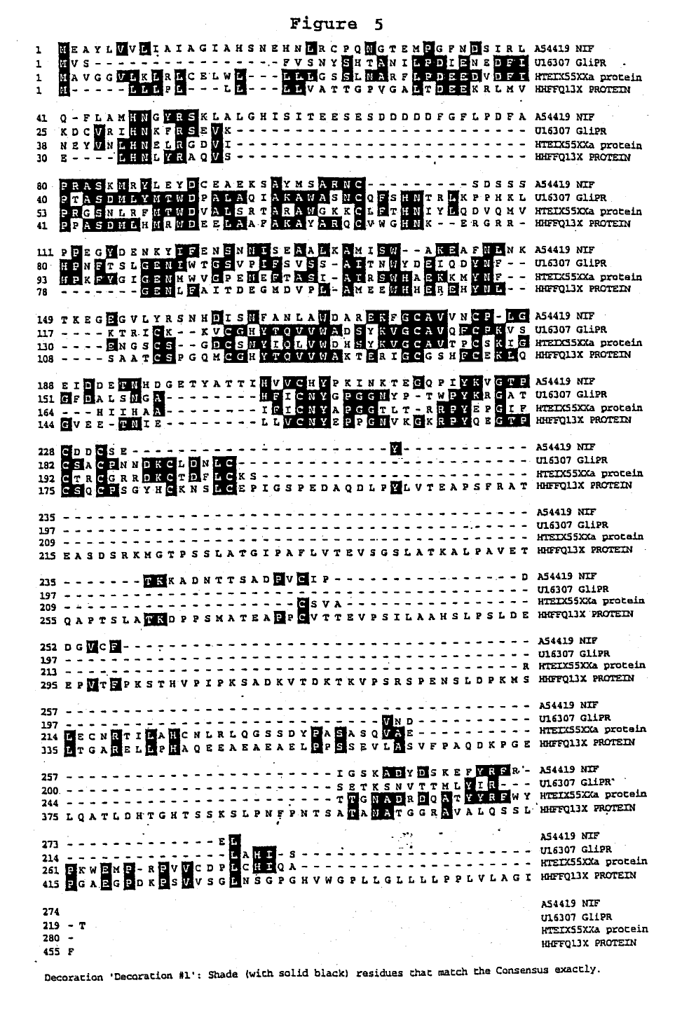

The determined nucleotide sequence of the LRF-1 cDNA of FIGS. 1A and 1B (SEQ ID NO:1) contains an open reading frame encoding a protein of 279 amino acid residues, including an initiation codon encoding an N-terminal methionine at nucleotide positions 31-33, and a predicted molecular weight of about 32 kDa. The amino acid sequence of the LRF-1 protein shown in SEQ ID NO:2 shares about 18.5% sequence identity and about 45.1% sequence similarity (as determined by Bestfit using default parameters, see FIG. 3) to the amino acid sequence of the protein called “Neutrophil Inhibitory Factor (NIF)” (SEQ ID NO:5), the 41-kilodalton glycoprotein isolated from the canine hookworm (Ancylostoma caninum) that potently inhibits CD11/CD18-dependent neutrophil function in vitro (Moyle, M., et al., supra) which can be accessed on GenBank as Accession No. A54419. The determined nucleotide sequence of the LRF-2 cDNA of FIGS. 2A, 2B, 2C, and 2D (SEQ ID NO:3) contains an open reading frame encoding a protein of 463 amino acid residues, including an initiation codon encoding an N-terminal methionine at nucleotide positions 10-12, and a predicted molecular weight of about 50 kDa. The amino acid sequence of the LRF-2 protein shown in SEQ ID NO:4 shares about 22.8% sequence identity and about 45.7% sequence similarity (as determined by Bestfit using default parameters, see, FIG. 4) to the amino acid sequence of the NIF protein (SEQ ID NO:5).

As one of ordinary skill would appreciate, due to the possibilities of sequencing errors discussed above, the actual complete LRF-1 polypeptide encoded by the deposited cDNA, which comprises about 279 amino acids, may be somewhat longer or shorter. Similarly, the actual complete LRF-2 polypeptide encoded by the deposited cDNA, which comprises about 463 amino acids, may be somewhat longer or shorter. Thus, the actual open reading frame of either deposited clone may be anywhere in the range of ±20 amino acids, more likely in the range of 110 amino acids, of that predicted from the initiating methionine codon shown in either FIGS. 1A and 1B or FIGS. 2A, 2B, 2C, and 2D. It will further be appreciated that, depending on the analytical criteria used for identifying various functional domains, the exact “address” of the transmembrane domain of the LRF-2 polypeptide may differ slightly from the predicted positions above. In any event, as discussed further below, the invention further provides polypeptides having various residues deleted from the N-terminus of the complete polypeptide, including polypeptides lacking one or more amino acids from the N-terminus of the mature domains described herein, which constitute additional forms of the LRF-1 and LRF-2 proteins of the invention.

Leader and Mature Sequences

The amino acid sequences of the complete LRF-1 and LRF-2 proteins include secretory leader sequences and related mature (secreted) protein forms, as shown in FIGS. 1A and 1B and FIGS. 2A, 2B, 2C, and 2D. More in particular, the present invention provides nucleic acid molecules encoding a mature form of the LRF-1 or LRF-2 protein. Thus, according to the signal hypothesis, once export of the growing protein chain across the rough endoplasmic reticulum has been initiated, proteins secreted by mammalian cells have a signal or secretory leader sequence which is cleaved from the complete polypeptide to produce a secreted “mature” form of the protein. Most mammalian cells and even insect cells cleave secreted proteins with the same specificity. However, in some cases, cleavage of a secreted protein is not entirely uniform, which results in two or more mature species of the protein. Further, it has long been known that the cleavage specificity of a secreted protein is ultimately determined by the primary structure of the complete protein, that is, it is inherent in the amino acid sequence of the polypeptide. Therefore, the present invention provides a nucleotide sequence encoding the mature LRF-1 polypeptide having the amino acid sequence encoded by the cDNA clone contained in the host identified as ATCC Deposit No.97860 as well as a nucleotide sequence encoding the mature LRF-2 polypeptide having the amino acid sequence encoded by the cDNA clone contained in the host identified as ATCC Deposit No. 97867. By, for instance, the “mature LRF-1 polypeptide having the amino acid sequence encoded by the cDNA clone in ATCC Deposit No. 97806” is meant the mature form(s) of the LRF-1 protein produced by expression in a eukaryotic cell (preferably a mammalian cell, e.g., COS cells, as described below) of the complete open reading frame encoded by the human DNA sequence of the clone contained in the vector in the deposited host.

In addition, methods for predicting whether a protein has a secretory leader as well as the cleavage point for that leader sequence are available. For instance, the method of McGeoch (Virus Res. 3:271-286 (1985)) uses the information from a short N-terminal charged region and a subsequent uncharged region of the complete (uncleaved) protein. The method of von Heinje (Nucleic Acids Res. 14:4683-4690 (1986)) uses the information from the residues surrounding the cleavage site, typically residues −13 to +2 where +1 indicates the amino terminus of the mature protein. The accuracy of predicting the cleavage points of known mammalian secretory proteins for each of these methods is in the range of 75-80% (von Heinje, supra). However, the two methods do not always produce the same predicted cleavage point(s) for a given protein.

In the present case, the deduced amino acid sequence of the complete LRF-1 polypeptide was analyzed by a computer program (“PSORT”, available from Dr. Kenta Nakai of the Institute for Chemical Research, Kyoto University (see K. Nakai and M. Kanehisa, Genomics 14.897-911 (1992)), which is an expert system for predicting the cellular location of a protein based on the amino acid sequence. As part of this computational prediction of localization, the methods of McGeoch and von Heinje are incorporated. The analysis of the LRF-1 amino acid sequence by this program, as well as using other similar analytical methods, led to the prediction of a single leader sequence cleavage site between amino acids 25 and 26 in FIGS. 1A and 1B (positions −3 and −2 in the complete amino acid sequence shown in SEQ ID NO:2). Comparable analyses of the LRF-2 amino acid sequence by this program and other similar analytical methods led to the prediction of two possible leader sequence cleavage sites, one between amino acids 20 and 21 in FIGS. 2A, 2B, 2C, and 2D (positions −1 and +1 in the complete amino acid sequence shown in SEQ ID NO:4), and the other between amino acids 22 and 23 in FIGS. 2A, 2B, 2C, and 2D (positions −1 and +1 in SEQ ID NO:4). As one of ordinary skill would appreciate from the above discussions, due to the possibilities of sequencing errors as well as the variability of cleavage sites in different known proteins, the mature LRF-1 polypeptide encoded by the deposited cDNA is expected to consist of about 253 amino acids (presumably residues 1 to 253 of SEQUENCE ID NO:2, but may consist of any number of amino acids in the range of about 243 to about 263 amino acids; and the actual leader sequence(s) of this protein is expected to be correspondingly longer or shorter, i.e. about 10 to about 30 amino acids (presumably residues −20 through −1 of SEQ ID NO:2). Similarly, the mature LRF-2 polypeptide encoded by the deposited cDNA is expected to consist of about 441 to 443 amino acids (presumably residues −2 to 441 or 1 to 441 of SEQ ID NO:4, but may consist of any number of amino acids in the range of about 431 to about 451 amino acids

As indicated, nucleic acid molecules of the present invention may be in the form of RNA, such as mRNA, or in the form of DNA, including, for instance, cDNA and genomic DNA obtained by cloning or produced synthetically. The DNA may be double-stranded or single-stranded. Single-stranded DNA or RNA may be the coding strand, also known as the sense strand, or it may be the non-coding strand, also referred to as the anti-sense strand.

By “isolated” nucleic acid molecule(s) is intended a nucleic acid molecule, DNA or RNA, which has been removed from its native environment For example, recombinant DNA molecules contained in a vector are considered isolated for the purposes of the present invention. Further examples of isolated DNA molecules include recombinant DNA molecules maintained in heterologous host cells or purified (partially or substantially) DNA molecules in solution. Isolated RNA molecules include in vivo or in vitro RNA transcripts of the DNA molecules of the present invention. Isolated nucleic acid molecules according to the present invention further include such molecules produced synthetically.

Isolated nucleic acid molecules of the present invention include DNA molecules comprising an open reading frame (ORF) with an initiation codon at positions 33-36 of the nucleotide sequence shown in FIGS. 1A and 1B (SEQ ID NO: 1), or DNA molecules comprising an ORF with an initiation codon at positions 10-12 of the nucleotide sequence shown in FIGS. 2A, 2B, 2C, and 2D (SEQ ID NO:3). Also included are DNA molecules comprising the coding sequence for the predicted mature LRF-1 and LRF-2 proteins shown respectively in FIGS. 1A and 1B and FIGS. 2A, 2B, 2C, and 2D (respectively, SEQ ID NOs:2 and 4).

In addition, isolated nucleic acid molecules of the invention include DNA molecules which comprise a sequence substantially different from those described above but which, due to the degeneracy of the genetic code, still encode the LRF-1 or LRF-2 protein. Of course, the genetic code and species-specific codon preferences are well known in the art. Thus, it would be routine for one skilled in the art to generate the degenerate variants described above, for instance, to optimize codon expression for a particular host (e.g., change codons in the human mRNA to those preferred by a bacterial host such as E. coli).

In another aspect, the invention provides isolated nucleic acid molecules encoding the LRF-1 or LRF-2 polypeptide having an amino acid sequence encoded by the cDNA clone contained in the plasmid deposited as, respectively, ATCC Deposit No. 97860 or ATCC Deposit No. 97867. Preferably, this nucleic acid molecule will encode the mature polypeptide encoded by each above-described deposited cDNA clone.

The invention further provides an isolated nucleic acid molecule having the nucleotide sequence shown in FIGS. 1A and 1B and FIGS. 2A, 2B, 2C, and 2D (SEQ ID NOs:1 or 2) or the nucleotide sequence of a cDNA contained in one of the above-described deposited clones, or a nucleic acid molecule having a sequence complementary to one of the above sequences. Such isolated molecules, particularly DNA molecules, are useful as probes for gene mapping, by in situ hybridization with chromosomes, and for detecting expression of the LRF-1 gene in human tissue, for instance, by Northern blot analysis.

The present invention is further directed to nucleic acid molecules encoding portions of the nucleotide sequences described herein as well as to fragments of the isolated nucleic acid molecules described herein. In particular, the invention provides a polynucleotide having a nucleotide sequence representing the portion of SEQ ID NOs: 1 or 2 which consists of the complete ORF (i.e., positions 31-867 of SEQ ID NO:1 or positions 10-1398 of SEQ ID NO:3).

In addition, the invention provides nucleic acid molecules having nucleotide sequences related to extensive portions of SEQ ID NO:1 which have been determined from the following related cDNA clones: HTEIX55R (SEQ ID NO:7). The invention also provides nucleic acid molecules having nucleotide sequences related to extensive portions of SEQ ID NO:3 which have been determined from the following related cDNA clones: HJAAR51 (SEQ ID NO:8); HARAZ76 (SEQ ID NO:9); HRDBF59 (SEQ ID NO:10); and HJABC86 (SEQ ID NO:11).

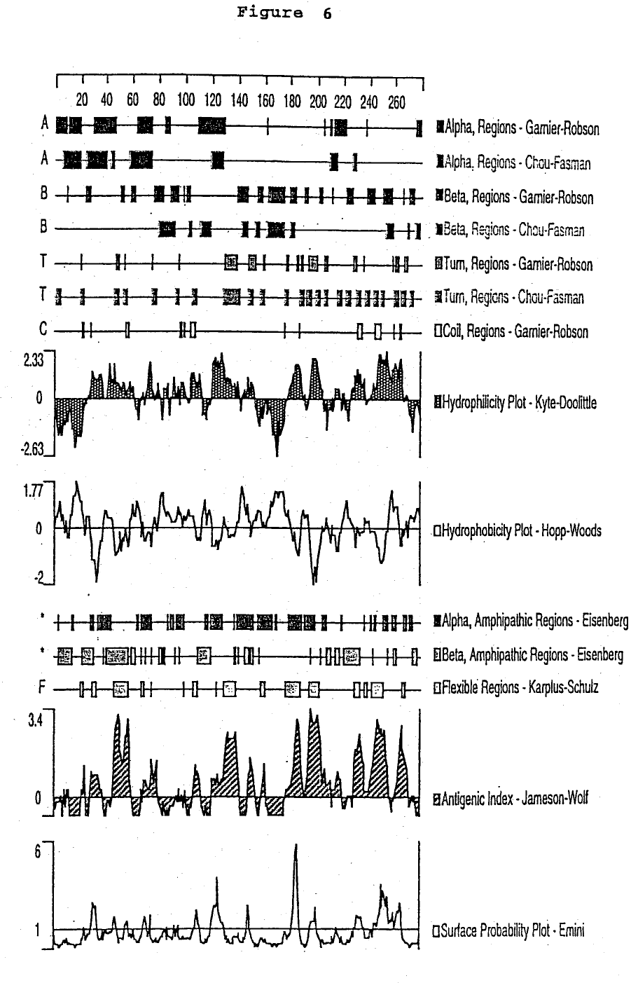

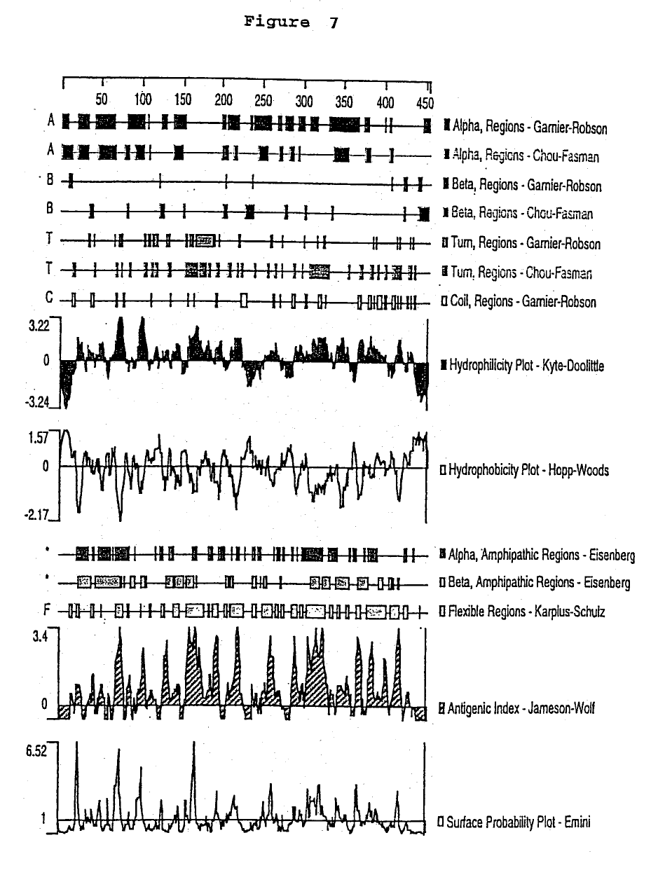

More generally, by a fragment of an isolated nucleic acid molecule having the nucleotide sequence of the deposited cDNA or the nucleotide sequence shown in FIG. 1 (SEQ ID NO: 1 or 3) is intended fragments at least about 15 nt, and more preferably at least about 20 nt, still more preferably at least about 30 nt, and even more preferably, at least about 40 nt in length which are useful as diagnostic probes and primers as discussed herein. Of course, larger fragments 50-300 nt in length are also useful according to the present invention as are fragments corresponding to most, if not all, of the nucleotide sequence of the deposited cDNA or as shown in FIGS. 1A and 1B and FIGS. 2A, 2B, 2C, and 2D (SEQ ID NO:1 or 3). By a fragment at least 20 nt in length, for example, is intended fragments which include 20 or more contiguous bases from the nucleotide sequence of the deposited cDNA or the nucleotide sequence as shown in FIGS. 1A and 1B and FIGS. 2A, 2B, 2C, and 2D (SEQ ID NO:1 or 3). Preferred nucleic acid fragments of the present invention include nucleic acid molecules encoding epitope-bearing portions of the LRF-1 or LRF-2 polypeptide as identified in FIGS. 6 and 7 and described in more detail below.

In another aspect, the invention provides an isolated nucleic acid molecule comprising a polynucleotide which hybridizes under stringent hybridization conditions to a portion of the polynucleotide in a nucleic acid molecule of the invention described above, for instance, the cDNA clone contained in one of the ATCC Deposits cited above. By “stringent hybridization conditions” is intended overnight incubation at 42° C. in a solution comprising: 50% formamide, 5×SSC (150 mM NaCl, 15 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at about 65° C.

By a polynucleotide which hybridizes to a “portion” of a polynucleotide is intended a polynucleotide (either DNA or RNA) hybridizing to at least about 15 nucleotides (nt), and more preferably at least about 20 nt, still more preferably at least about 30 nt, and even more preferably about 30-70 (e.g., 50) nt of the reference polynucleotide. These are useful as diagnostic probes and primers as discussed above and in more detail below.

By a portion of a polynucleotide of “at least 20 nt in length,” for example, is intended 20 or more contiguous nucleotides from the nucleotide sequence of the reference polynucleotide (e.g., the deposited cDNA or the nucleotide sequence as shown in FIGS. 1A and 1B and FIGS. 2A, 2B, 2C, and 2D (SEQ ID NO: 1 or 3)). Of course, a polynucleotide which hybridizes only to a poly A sequence (such as the 3′ terminal poly(A) tract of the LRF-1 cDNA shown in FIGS. 1A and 1B and FIGS. 2A, 2B, 2C, and 2D (SEQ ID NO:1 of 3)), or to a complementary stretch of T (or U) residues, would not be included in a polynucleotide of the invention used to hybridize to a portion of a nucleic acid of the invention, since such a polynucleotide would hybridize to any nucleic acid molecule containing a poly (A) stretch or the complement thereof (e.g., practically any double-stranded cDNA clone).

As noted, nucleic acids of the invention may encode the complete amino acid sequence of an LRF-1 or LRF-2 polypeptide, or a portion thereof. Also encoded by nucleic acids of the invention are the above protein sequences together with additional, non-coding sequences, including for example, but not limited to introns and non-coding 5′ and 3′ sequences, such as the transcribed, non-translated sequences that play a role in transcription, mRNA processing, including splicing and polyadenylation signals, for example—ribosome binding and stability of mRNA; an additional coding sequence which codes for additional amino acids, such as those which provide additional functionalities.

Thus, the sequence encoding the polypeptide may be fused to a marker sequence, such as a sequence encoding a peptide which facilitates purification of the fused polypeptide. In certain preferred embodiments of this aspect of the invention, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86:821-824 (1989), for instance, hexa-histidine provides for convenient purification of the fusion protein. The “HA” tag is another peptide useful for purification which corresponds to an epitope derived from the influenza hemagglutinin protein, which has been described by Wilson et al., Cell 37: 767 (1984). As discussed below, other such fusion proteins include the LRF-1 fused to Fe at the N- or C-terminus.

Variant and Mutant Polynucleotides

The present invention further relates to variants of the nucleic acid molecules of the present invention, which encode portions, analogs or derivatives of the LRF-1 protein. Variants may occur naturally, such as a natural allelic variant. By an “allelic variant” is intended one of several alternate forms of a gene occupying a given locus on a chromosome of an organism. Genes II, Lewin, B., ed., John Wiley & Sons, New York (1985). Non-naturally occurring variants may be produced using art-known mutagenesis techniques.

Such variants include those produced by nucleotide substitutions, deletions or additions. The substitutions, deletions or additions may involve one or more nucleotides. The variants may be altered in coding regions, non-coding regions, or both. Alterations in the coding regions may produce conservative or non-conservative amino acid substitutions, deletions or additions. Especially preferred among these are silent substitutions, additions and deletions, which do not alter the properties and activities of the LRF-1 or LRF-2 proteins or portions thereof. Also especially preferred in this regard are conservative substitutions. Most highly preferred are nucleic acid molecules encoding the mature protein having the amino acid sequence shown in SEQ ID NO:2 or 4, or the mature LRF-1 or LRF-2 amino acid sequences encoded by the respective deposited cDNA clones.

Further embodiments include an isolated nucleic acid molecule comprising a polynucleotide having a nucleotide sequence at least 90% identical, and more preferably at least 95%, 96%, 97%, 98% or 99% identical to a polynucleotide selected from the group consisting of: (a) a nucleotide sequence encoding the LRF-1 polypeptide having the complete amino acid sequence in SEQ ID NO:2 excepting the N-terminal methionine (i.e., positions −24 to +254 of SEQ ID NO:2); (b) a nucleotide sequence encoding the predicted mature LRF-1 polypeptide having the amino acid sequence at positions 1-254 in SEQ ID NO:2; (c) a nucleotide sequence encoding the LRF-1 polypeptide having the complete amino acid sequence excepting the N-terminal methionine encoded by the cDNA clone contained in ATCC Deposit No 97860; (d) a nucleotide sequence encoding the mature LRF-1 polypeptide having the amino acid sequence encoded by the cDNA clone contained in ATCC Deposit No. 97860; and (e) a nucleotide sequence complementary to any of the nucleotide sequences in (a), (b), (c) or (d) above.

Another aspect of the invention provides an isolated nucleic acid molecule comprising a polynucleotide comprising a nucleotide sequence at least 90% identical, and more preferably at least 95%, 96%, 97%, 98% or 99% identical to a polynucleotide selected from the group consisting of: (a) a nucleotide sequence encoding the LRF-2 polypeptide having the complete amino acid sequence in SEQ ID NO:4 excepting the N-terminal methionine (i.e., positions −21 to 441 of SEQ ID NO:4); (b) a nucleotide sequence encoding the predicted mature LRF-2 polypeptide having the amino acid sequence at positions −2 to 441 or at positions 1 to 441 in SEQ ID NO:4; (c) a nucleotide sequence encoding the predicted soluble mature LRF-2 polypeptide having the amino acid sequence at about position −2 to about position 418 or at about position 1 to about position 418 in SEQ ID NO:4; (d) a nucleotide sequence encoding the LRF-2 polypeptide having the complete amino acid sequence excepting the N-terminal methionine encoded by the cDNA clone contained in ATCC Deposit No 97867; (e) a nucleotide sequence encoding the mature LRF-2 polypeptide having the amino acid sequence encoded by the cDNA clone contained in ATCC Deposit No. 97867; (f) a nucleotide sequence encoding the soluble mature LRF-2 polypeptide having the amino acid sequence of the mature LRF-2 polypeptide encoded by the cDNA clone contained in ATCC Deposit No. 97867 excepting the C-terminal sequence of about 23 amino acids of the mature LRF-2 polypeptide encoded by that CDNA; and (g) a nucleotide sequence complementary to any of the nucleotide sequences in (a), (b), (c), (d), (e) or (f) above.

By a polynucleotide having a nucleotide sequence at least, for example, 95% “identical” to a reference nucleotide sequence encoding an LRF-1 or LRF-2 polypeptide is intended that the nucleotide sequence of the polynucleotide is identical to the reference sequence except that the polynucleotide sequence may include up to five point mutations per each 100 nucleotides of the reference nucleotide sequence encoding the LRF-1 polypeptide. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence.

As a practical matter, whether any particular nucleic acid molecule is at least 90%, 95%, 96%, 97%, 98% or 99% identical to, for instance, the nucleotide sequence shown in FIGS. 1A and 1B and FIGS. 2A, 2B, 2C, and 2D or to the nucleotide sequences of the deposited cDNA clones can be determined conventionally using known computer programs such as the Bestfit program (Wisconsin Sequence Analysis Package, Version 8 for Unix, Genetics Computer Group, University Research Park, 575 Science Drive, Madison, Wis. 53711). Bestfit uses the local homology algorithm of Smith and Waterman, Advances in Applied Mathematics 2:482-489 (1981), to find the best segment of homology between two sequences. When using Bestfit or any other sequence alignment program to determine whether a particular sequence is, for instance, 95% identical to a reference sequence according to the present invention, the parameters are set, of course, such that the percentage of identity is calculated over the full length of the reference nucleotide sequence and that gaps in homology of up to 5% of the total number of nucleotides in the reference sequence are allowed.

The present application is directed to nucleic acid molecules at least 90%, 95%, 96%, 97%, 98% or 99% identical to a nucleic acid sequence shown in FIGS. 1A and 1B (SEQ ID NO:1) or to a nucleic acid sequence of the deposited cDNAs, irrespective of whether they encode a polypeptide having LRF-1 or LRF-2 activity. This is because even where a particular nucleic acid molecule does not encode a polypeptide having LRF-1 or LRF-2 activity, one of skill in the art would still know how to use the nucleic acid molecule, for instance, as a hybridization probe or a polymerase chain reaction (PCR) primer. Uses of the nucleic acid molecules of the present invention that do not encode a polypeptide having LRF-1 or LRF-2 activity include, inter alia, (1) isolating the LRF-1 or LRF-2 gene or allelic variants thereof in a cDNA library; (2) in situ hybridization (e.g., “FISH”) to metaphase chromosomal spreads to provide precise chromosomal location of the LRF-1 or LRF-2 gene, as described in Verma et al., Human Chromosomes: A Manual of Basic Techniques, Pergamon Press, New York (1988); and Northern Blot analysis for detecting LRF-1 mRNA expression in specific tissues.

Preferred, however, are nucleic acid molecules having sequences at least 90%, 95%, 96%, 97%, 98% or 99% identical to the nucleic acid sequence shown in FIGS. 1A and 1B (SEQ ID NO:1) or to the nucleic acid sequence of the deposited cDNA which do, in fact, encode a polypeptide having LRF-1 protein activity. By “a polypeptide having LRF-1 (or LRF-2) activity” is intended polypeptides exhibiting activity similar, but not necessarily identical, to an activity of the mature LRF-1 (or LRF-2) protein of the invention, as measured in a particular biological assay. For example, the LRF-1 and LRF-2 proteins of the present invention modulate neutrophil adhesion. An in vitro assay for measuring the extent of inhibition (or stimulation) of adhesion of neutrophils is described, for instance, in Barnard, J. W., et al., supra. Briefly, the assay involves coating culture plates (e.g., Terasaki plates) with 1% gelatin, for instance, at room temperature for 30 min., and then seeding in those plates endothelial cells (e.g., primary HUVEC, second or third passage) suspended in endothelial cell growth medium containing M199, 20% FCS, 2 mM L-glutamine, and penicillin and streptomycin (Life Technologies, Grand Island, N.Y.). For instance, such cells may be seeded at a density of about 1 million cells/ml of medium, adding 10 microliters of medium containing cells per well and then filling the wells with more medium. After reaching confluence (e.g., about 2 days incubation at 37° C.), the isolated neutrophils are suspended in HBSS (1 million cells/ml) and activated by 30 ng/ml PMA for 15 min. at 37° C.). Then, 10 microliters of activated neutrophils are added per well of the endothelial monolayers. Plates are placed on ice for 30 min. to allow settling, and then warmed to 37° C. for 30 min. for adherence, after which the plates are washed with M199 three times and fixed in 0.1% paraformaldahyde. Adherent neutrophils are counted by phase contrast microscopy in quadruplicate wells.

Other assays which may be used to measure activity of proteins in modulating neutrophil functions include, for example, assays measuring adhesion of neutrophils to fibrinogen, effects of neutrophils on transendothelial albumin permeability, and, ex vivo neutrophil-dependent lung vascular injury (indicated by, e.g., edema and neutrophil uptake). See, for instance, Barnard, J. W., et al., supra. Neuroprotective activity of LRF-1 and LRF-2 polypeptides may be determined in a model of focal cerebral ischemia in the rat. See Jiang, N., et al., supra. Other neutrophil modulating activities of LRF-1 and LRF-2 polypeptides which can be measured by know methods include attenuation of the inflammatory response in an animal colitis (see, for example, Meenan, J., et al., supra) and reduction of leukocyte adhesion in the liver after hemorrhagic shock (e.g., Bauer, C., et al., supra. Methods suitable for characterization of the interaction of LRF-1 and LRF-2 polypeptides with integrin receptors are described, for instance, by Rieu, P. et al, supra.

LRF-1 and LRF-2 polypeptides modulate leukocyte functions in a dose-dependent manner in the above-described assays. Thus, “a polypeptide having LRF-1 (or LRF-2) protein activity” includes polypeptides that also exhibit any of the same leukocyte modulating activities in the above-described assays in a dose-dependent manner. Although the degree of dose-dependent activity need not be identical to that of the mature LRF-1 or LRF-2 protein, preferably, “a polypeptide having LRF-1 (or LRF-2) protein activity” will exhibit substantially similar dose-dependence in a given activity as compared to the mature LRF-1 (or LRF-2) protein (i.e., the candidate polypeptide will exhibit greater activity or not more than about 25-fold less and, preferably, not more than about tenfold less activity relative to the reference LRF-1 of LRF-2 protein).

Of course, due to the degeneracy of the genetic code, one of ordinary skill in the art will immediately recognize that a large number of the nucleic acid molecules having a sequence at least 90%, 95%, 96%, 97%, 98%, or 99% identical to the nucleic acid sequence of the deposited cDNA or the nucleic acid sequence shown in FIGS. 1A and 1B (SEQ ID NO:1) will encode a polypeptide “having LRF-1 (or LRF-2) protein activity.” In fact, since degenerate variants of these nucleotide sequences all encode the same polypeptide, this will be clear to the skilled artisan even without performing the above described comparison assay. It will be further recognized in the art that, for such nucleic acid molecules that are not degenerate variants, a reasonable number will also encode a polypeptide having LRF-1 or LRF-2 protein activity. This is because the skilled artisan is fully aware of amino acid substitutions that are either less likely or not likely to significantly effect protein function (e.g., replacing one aliphatic amino acid with a second aliphatic amino acid), as further described below.

Vectors and Host Cells

The present invention also relates to vectors which include the isolated DNA molecules of the present invention, host cells which are genetically engineered with the recombinant vectors, and the production of LRF-1 or LRF-2 polypeptides or fragments thereof by recombinant techniques. The vector may be, for example, a phage, plasmid, viral or retroviral vector. Retroviral vectors may be replication competent or replication defective. In the latter case, viral propagation generally will occur only in complementing host cells.

The polynucleotides may be joined to a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells.

The DNA insert should be operatively linked to an appropriate promoter, such as the phage lambda PL promoter, the E. coli lac, trp, phoA and tac promoters, the SV40 early and late promoters and promoters of retroviral LTRs, to name a few. Other suitable promoters will be known to the skilled artisan. The expression constructs will further contain sites for transcription initiation, termination and, in the transcribed region, a ribosome binding site for translation. The coding portion of the transcripts expressed by the constructs will preferably include a translation initiating codon at the beginning and a termination codon (UAA, UGA or UAG) appropriately positioned at the end of the polypeptide to be translated.

As indicated, the expression vectors will preferably include at least one selectable marker. Such markers include dihydrofolate reductase, G418 or neomycin resistance for eukaryotic cell culture and tetracycline, kanamycin or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells; insect cells such as Drosophila S2 and Spodoptera Sf9 cells; animal cells such as CHO, COS, 293 and Bowes melanoma cells; and plant cells. Appropriate culture mediums and conditions for the above-described host cells are known in the art.

Among vectors preferred for use in bacteria include pQE70, pQE60 and pQE-9, available from QIAGEN, Inc., supra; pBS vectors, Phagescript vectors, Bluescript vectors, pNH8A, pNH 16a, pNH 18A, pNH46A, available from Stratagene; and ptrc99a, pKK223-3, pKK233-3, pDR540, pRIT5 available from Pharmacia. Among preferred eukaryotic vectors are pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986).

The polypeptide may be expressed in a modified form, such as a fusion protein, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties may be added to the polypeptide to facilitate purification. Such regions may be removed prior to final preparation of the polypeptide. The addition of peptide moieties to polypeptides to engender secretion or excretion, to improve stability and to facilitate purification, among others, are familiar and routine techniques in the art. A preferred fusion protein comprises a heterologous region from immunoglobulin that is useful to stabilize and purify proteins. For example, EP-A-O 464 533 (Canadian counterpart 2045869) discloses fusion proteins comprising various portions of constant region of immunoglobulin molecules together with another human protein or part thereof. In many cases, the Fe part in a fusion protein is thoroughly advantageous for use in therapy and diagnosis and thus results, for example, in improved pharmacokinetic properties (EP-A 0232 262). On the other hand, for some uses it would be desirable to be able to delete the Fe part after the fusion protein has been expressed, detected and purified in the advantageous manner described. This is the case when Fe portion proves to be a hindrance to use in therapy and diagnosis, for example when the fusion protein is to be used as antigen for immunizations. In drug discovery, for example, human proteins, such as hIL-5, have been fused with Fe portions for the purpose of high-throughput screening assays to identify antagonists of hIL-5. See, D. Bennett et al., J. Molecular Recognition 8:52-58 (1995) and K. Johanson et al., J. Biol. Chem. 270:9459-9471 (1995).

The LRF-1 or LRF-2 protein can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification. Polypeptides of the present invention include: products purified from natural sources, including bodily fluids, tissues and cells, whether directly isolated or cultured; products of chemical synthetic procedures; and products produced by recombinant techniques from a prokaryotic or eukaryotic host, including, for example, bacterial, yeast, higher plant, insect and mammalian cells. Depending upon the host employed in a recombinant production procedure, the polypeptides of the present invention may be glycosylated or may be non-glycosylated. In addition, polypeptides of the invention may also include an initial modified methionine residue, in some cases as a result of host-mediated processes. Thus, it is well known in the art that the N-terminal methionine encoded by the translation initiation codon generally is removed with high efficiency from any protein after translation in all eukaryotic cells. While the N-terminal methionine on most proteins also is efficiently removed in most prokaryotes, for some proteins this prokaryotic removal process is inefficient, depending on the nature of the amino acid to which the N-terminal methionine is covalently linked.

Polypeptides and Fragments

The invention further provides an isolated LRF-1 of LRF-2 polypeptide having the amino acid sequence encoded by the deposited cDNA, or the amino acid sequence, respectively, in SEQ ID NO:2 or SEQ ID NO:4, or a peptide or polypeptide comprising a portion of the above polypeptides. Variant and Mutant Polypeptides

To improve or alter the characteristics of LRF-1 or LRF-2 polypeptides, protein engineering may be employed. Recombinant DNA technology known to those skilled in the art can be used to create novel mutant proteins or “muteins including single or multiple amino acid substitutions, deletions, additions or fusion proteins. Such modified polypeptides can show, e.g., enhanced activity or increased stability. In addition, they may be purified in higher yields and show better solubility than the corresponding natural polypeptide, at least under certain purification and storage conditions.

N-Terminal and C-Terminal Deletion Mutants

For many proteins, including the extracellular domain of a membrane associated protein or the mature form(s) of a secreted protein, it is known in the art that one or more amino acids may be deleted from the N-terminus or C-terminus without substantial loss of biological function. For instance, Ron et al., J. Biol. Chem., 268:2984-2988 (1993) reported modified KGF proteins that had heparin binding activity even if 3, 8, or 27 amino-terminal amino acid residues were missing. In the present case, since the proteins of the invention are related to CRISP proteins which are known to have multiple disulfide bridges between cysteines, deletions of N-terminal amino acids up to the first cysteine which is conserved in all the sequences of LRF-1, LRF-2, NIF (Neutrophil Inhibitory Factor; Moyle, M., et al., supra), and the human GliPR (glioma pathogenesis-related protein; Murphy, E. V., et al., supra). See, FIG. 5. Therefore, in LRF-1 and LRF-2, N-terminal deletions up to this first conserved cysteine residue (at LRF-1 position 78 in FIGS. 1A and 1B and position 53 in SEQ ID NO:2; at LRF-2 position 74 in FIGS. 2A, 2B, 2C, and 2D and position 52 in SEQ ID NO:4) may retain some biological activity such as receptor binding.

However, even if deletion of one or more amino acids from the N-terminus of a protein results in modification or loss of one or more biological functions of the protein, other biological activities may still be retained. Thus, the ability of the shortened protein to induce and/or bind to antibodies which recognize the complete or mature form of the protein generally will be retained when less than the majority of the residues of the complete mature protein are removed from the N-terminus. Whether a particular polypeptide lacking N-terminal residues of a complete protein retains such immunologic activities can readily be determined by routine methods described herein and otherwise known in the art.

Accordingly, the present invention further provides polypeptides having one or more residues deleted from the amino terminus of the amino acid sequence of the LRF-1 shown in SEQ ID NO:2, up to the Cys residue at position number 53, and polynucleotides encoding such polypeptides. In particular, the present invention provides polypeptides comprising the amino acid sequence of residues n-254 of SEQ ID NO:2, where n is an integer in the range of −24 to +53. More in particular, the invention provides polynucleotides encoding polypeptides having the amino acid sequence of residues of −24 to +254, −23 to +254, −22 to +254, −21 to +254, −20 to +254, −19 to +254, −18 to +254, −17 to +254, −16 to +254, −15 to +254, −14 to +254, −13 to +254, −12 to +254, −11 to +254, −10 to +254, −9 to +254, −8 to +254, −7 to +254, −6 to +254, −5 to +254, −4 to +254, −3 to +254, −2 to +254, −1 to +254, +1 to +254, +2 to +254, +3 to +254, +4 to +254, +5 to +254, +5 to +254, +5 to +254, +5 to +254, +5 to +254, +5 to +254, +5 to +254, +6 to +254, +6 to +254, +7 to +254, +8 to +254, +9 to +254, +10 to +254, +11 to +254, +12 to +254, +13 to +254, +14 to +254, +15 to +254, +16 to +254, +17 to +254, +18 to +254, +19 to +254, +20 to +254, +21 to +254, +22 to +254, +23 to +254, +24 to +254, +25 to +254, +26 to +254, +27 to +254, +28 to +254, +29 to +254, +30 to +254, +31 to +254, +32 to +254, +33 to +254, +34 to +254, +35 to +254, +36 to +254, +37 to +254, +38 to +254, +39 to +254, +40 to +254, +41 to +254, +42 to +254, +43 to +254, +44 to +254, +45 to +254, +46 to +254, +47 to +254, +48 to +254, +49 to +254, +50 to +254, +51 to +254, +52 to +254 and +53 to +254 of SEQ ID NO:2. Polynucleotides encoding these polypeptides also are provided.

Similarly, the present invention provides polypeptides having one or more residues deleted from the amino terminus of the amino acid sequence of the LRF-2 shown in SEQ ID NO:4, up to the Cys residue at position number 52, and polynucleotides encoding such polypeptides. In particular, the present invention provides polypeptides comprising the amino acid sequence of residues n-443 of SEQ ID NO:4, where n is an integer in the range of −21 to +52. More in particular, the invention provides polynucleotides encoding polypeptides having the amino acid sequence of residues −19 to +441, −18 to +441, −17 to +441, −16 to +441, −15 to +441, −14 to +441, −13 to +441, −12 to +441, −11 to +441, −10 to +441, −9 to +441, −8 to +441, −7 to +441, −6 to +441, −5 to +441, −4 to +441, −3 to +441, −2 to +441, −1 to +441, +1 to +441, +2 to +441, +3 to +441, +4 to +441, +5 to +441, +5 to +441, +5 to +441, +5 to +441, +5 to +441, +5 to +441, +5 to +441, +6 to +441, +6 to +441, +7 to +441, +8 to +441, +9 to +441, +10 to +441, +11 to +441, +12 to +441, +13 to +441, +14 to +441, +15 to +441, +16 to +441, +17 to +441, +18 to +441, +19 to +441, +20 to +441, +21 to +441, +22 to +441, +23 to +441, +24 to +441, +25 to +441, +26 to +441, +27 to +441, +28 to +441, +29 to +441, +30 to +441, +31 to +441, +32 to +441, +33 to +441, +34 to +441, +35 to +441, +36 to +441, +37 to +441, +38 to +441, +39 to +441, +40 to +441, +41 to +441, +42 to +441, +43 to +441, +44 to +441, +45 to +441, +46 to +441, +47 to +441, +48 to +441, +49 to +441, +50 to +441, +51 to +441, and +52 to +441 of SEQ ID NO:4. Polynucleotides encoding these polypeptides also are provided.

Further, many examples of biologically functional C-terminal deletion muteins are known. For instance, interferon gamma shows up to ten times higher activities by deleting 8-10 amino acid residues from the carboxy terminus of the protein (Döbeli et al., J. Biotechnology 7:199-216 (1988). In the present case, since the proteins of the invention are related to CRISP proteins which are known to have multiple disulfide bridges between cysteines, deletions of C-terminal amino acids up to the last (C-terminal) cysteine conserved in the LRF-1 and NIF sequences (LRF-1 position 209 in FIGS. 1A and 1B (position 184 in SEQ ID NO:2; See, FIG. 3 for alignment of LRF-1 and NIF sequences) may retain some biological activity such as receptor binding. However, even if deletion of one or more amino acids from the C-terminus of a protein results in modification or loss of one or more biological functions of the protein, other biological activities may still be retained. Thus, the ability of the shortened protein to induce and/or bind to antibodies which recognize the complete or mature form of the protein generally will be retained when less than the majority of the residues of the complete or mature protein are removed from the C-terminus. Whether a particular polypeptide lacking C-terminal residues of a complete protein retains such immunologic activities can readily be determined by routine methods described herein and otherwise known in the art.

Accordingly, the present invention further provides polypeptides having one or more residues deleted from the carboxy terminus of the amino acid sequence of LRF-1 shown in SEQ ID NO:2, up to the Cys residue at position 184 in SEQ ID NO:2, and polynucleotides encoding such polypeptides. In particular, the present invention provides polypeptides having the amino acid sequence of residues −25 to m of the amino acid sequence in SEQ ID NO:2, where m is any integer in the range of 184 to 253. More in particular, the invention provides polynucleotides encoding polypeptides having the amino acid sequence of residues −24 to +184, −24 to +185, −24 to +186, −24 to +187, −24 to +188, −24 to +189, −24 to +190, −24 to +191, −24 to +192, −24 to +193, −24 to +194, −24 to +195, −24 to +196, −24 to +197, −24 to +198, −24 to +199, −24 to +200, −24 to +201, −24 to +202, −24 to +203, −24 to +204, −24 to +205, −24 to +206, −24 to +207, −24 to +208, −24 to +209, −24 to +210, −24 to +211, −24 to +212, −24 to +213, −24 to +214, −24 to +215, −24 to +216, −24 to +217, −24 to +218, −24 to +219, −24 to +220, −24 to +221, −24 to +222, −24 to +223, −24 to +224, −24 to +225, −24 to +226, −24 to +227, −24 to +228, −24 to +229, −24 to +230, −24 to +231, −24 to +232, −24 to +233, −24 to +234, −24 to +235, −24 to +266, −24 to +237, −24 to +238, −24 to +239, −24 to +240, −24 to +241, −24 to +242, −24 to +243, −24 to +244, −24 to +245, −24 to +246, −24 to +247, −24 to +248, −24 to +249, −24 to +250, −24 to +251 and −24 to +252 of SEQ ID NO:2. Polynucleotides encoding these polypeptides also are provided.

In the case of LRF-2, the complete amino acid sequence (SEQ ID NO:4) comprises about 463 amino acids compared to 279 amino acids in the LRF-2 sequence and 274 amino acids in the NIF sequence. As noted above the additional C-terminal sequence of LRF-2 includes a peroxidase “signature” sequence (i.e., the amino acid sequence EVPSILAAHSL at positions 287-297 of FIGS. 2A, 2B, 2C, and 2D (positions 265-275 of SEQ ID NO:4) and a hydrophobic C-terminal sequence comprising a predicted transmembrane domain of about 16 amino acids (i.e., the sequence PGHVMGPLLGLLLLPP (SEQ ID NO:24) underlined in FIGS. 2A, 2B, 2C, and 2D) comprising amino acid number about 441 to about 456 in FIGS. 2A, 2B, 2C, and 2D (positions 419 to 434 in SEQ ID NO:4). Deletions of the LRF-2 polypeptide from the C-terminal end which remove the transmembrane domain and the peroxidase signature, and up to the last (C-terminal) cysteine conserved in the LRF-2 and NIF sequences (LRF-2 position 186 in FIGS. 2A, 2B, 2C, and 2D (position 164 in SEQ ID NO:4; See, FIG. 4 for alignment of LRF-2 and NIF sequences) may retain some biological activity such as receptor binding. However, even if deletion of one or more amino acids from the C-terminus of a protein results in modification or loss of one or more biological functions of the protein, other biological activities may still be retained, as explained above.

Accordingly, the present invention further provides polypeptides having one or more residues deleted from the carboxy terminus of the amino acid sequence of LRF-2 shown in SEQ ID NO:4, up to about the Cys residue at position 164 in SEQ ID NO:4, and polynucleotides encoding such polypeptides. In particular, the present invention provides polypeptides having the amino acid sequence of residues −19 to m of the amino acid sequence in SEQ ID NO:4, where m is any integer in the range of 166 to 442. Particularly preferred are C-terminal deletions which remove the sequence up to and including the peroxidase signature at positions 287-297 of FIGS. 2A, 2B, 2C, and 2D (positions 265-279 of SEQ ID NO:4). More in particular, the invention provides polynucleotides encoding such preferred polypeptides having, for example, the amino acid sequence of residues −21 to +166, −21 to +167, −21 to +168, −21 to +169, −21 to +170, −21 to +171, −21 to +172, −21 to +173, −21 to +174, −21 to +175, −21 to +176, −21 to +177, −21 to +178, −21 to +179, −21 to +180, −21 to +181, −21 to +182, −21 to +183, −21 to +184, −21 to +185, −21 to +186, −21 to +187, −21 to +188, −21 to +189, −21 to +190, −21 to +191, −21 to +192, −21 to +193, −21 to +194, −21 to +195, −21 to +196, −21 to +197, −21 to +198, −21 to +199, −21 to +200, −21 to +201, −21 to +202, −21 to +203, −21 to +204, −21 to +205, −21 to +206, −21 to +207, −21 to +208, −21 to +209, −21 to +210, −21 to +211, −21 to +212, −21 to +213, −21 to +214, −21 to +215, −21 to +216, −21 to +217, −21 to +218, −21 to +219, −21 to +220, −21 to +221, −21 to +222, −21 to +223, −21 to +224, −21 to +225, −21 to +226, −21 to +227, −21 to +228, −21 to +229, −21 to +230, −21 to +231, −21 to +232, −21 to +233, −21 to +234, −21 to +235, −21 to +266, −21 to +237, −21 to +238, −21 to +239, −21 to +240, −21 to +241, −21 to +242, −21 to +243, −21 to +244, −21 to +245, −21 to +246, −21 to +247, −21 to +248, −21 to +249, −21 to +250, −21 to +251, −21 to +252, −21 to +253, −21 to +254, −21 to +255, −21 to +256, −21 to +257, −21 to +258, −21 to +259, −21 to +260, −21 to +261, −21 to +262, −21 to +263, and −21 to +264 of SEQ ID NO:4. Polynucleotides encoding these polypeptides also are provided.

Also preferred are C-terminal deletions which remove the amino acid sequence of LRF-2 up to and including all or part of the hydrophobic C-terminal sequence comprising a predicted transmembrane domain of about 16 amino acids (i.e., the sequence PGHVMGPLLGLLLLPP (SEQ ID NO:24) underlined in FIGS. 2A, 2B, 2C, and 2D) comprising amino acid numbers about 441 to about 456 in FIGS. 2A, 2B, 2C, and 2D (positions 419 to 434 in SEQ ID NO:4). More in particular, the invention provides polynucleotides encoding polypeptides having, for example, the amino acid sequence of residues −22 to +411, −22 to +412, −22 to +413, −22 to +414, −22 to +415, −22 to +416, −22 to +417, −22 to +418, −22 to +419, −22 to +420, −22 to +421, −22 to +423, −22 to +424, −22 to +425, −22 to +426, −22 to +427, −22 to +428, −22 to +429, −22 to +430, −22 to +431, −22 to +432, −22 to +433, −22 to +434, −22 to +435, −22 to +436, −22 to +437, −22 to +438, −22 to +439 and −22 to +440 of SEQ ID NO:4.

The invention also provides polypeptides having one or more amino acids deleted from both the amino and the carboxyl termini of the LRF-1 or LRF-2 amino acid sequence, which may be described generally as having residues n-m of SEQ ID NO:2 or SEQ ID NO:4, respectively, where n and m are integers as described above.

Also included are a nucleotide sequence encoding a polypeptide consisting of a portion of the complete LRF-1 amino acid sequence encoded by the cDNA clone contained in ATCC Deposit No. 97860, where this portion excludes from 1 to about 53 amino acids from the amino terminus of the complete amino acid sequence encoded by the cDNA clone contained in ATCC Deposit No. 97860, or from 1 to about 69 amino acids from the carboxy terminus, or any combination of the above amino terminal and carboxy terminal deletions, of the complete amino acid sequence encoded by the cDNA clone contained in ATCC Deposit No. 97860. Similarly included are a nucleotide sequence encoding a polypeptide consisting of a portion of the complete LRF-2 amino acid sequence encoded by the cDNA clone contained in ATCC Deposit No. 97867, where this portion excludes from 1 to about 65 amino acids from the amino terminus of the complete amino acid sequence encoded by the cDNA clone contained in ATCC Deposit No. 97867, or from 1 to about 277 amino acids from the carboxy terminus, or any combination of the above amino terminal and carboxy terminal deletions, of the complete amino acid sequence encoded by the cDNA clone contained in ATCC Deposit No. 97867. Polynucleotides encoding all of the above deletion mutant polypeptide forms also are provided.

Other Mutants

In addition to terminal deletion forms of the protein discussed above, it also will be recognized by one of ordinary skill in the art that some amino acid sequences of the LRF-1 and LRF-2 polypeptides can be varied without significant effect of the structure or function of the protein. If such differences in sequence are contemplated, it should be remembered that there will be critical areas on the protein which determine activity.

Thus, the invention further includes variations of the LRF-1 or LRF-2 polypeptide which show substantial LRF-1 of LRF-2 polypeptide activity or which include regions of the LRF-1 or LRF-2 protein such as the protein portions discussed below. Such mutants include deletions, insertions, inversions, repeats, and type substitutions selected according to general rules known in the art so as have little effect on activity. For example, guidance concerning how to make phenotypically silent amino acid substitutions is provided in Bowie, J. U., et al, “Deciphering the Message in Protein Sequences: Tolerance to Amino Acid Substitutions,!” Science 247:1306-1310 (1990), wherein the authors indicate that there are two main approaches for studying the tolerance of an amino acid sequence to change. The first method relies on the process of evolution, in which mutations are either accepted or rejected by natural selection. The second approach uses genetic engineering to introduce amino acid changes at specific positions of a cloned gene and selections or screens to identify sequences that maintain functionality.

As the authors state, these studies have revealed that proteins are surprisingly tolerant of amino acid substitutions. The authors further indicate which amino acid changes are likely to be permissive at a certain position of the protein. For example, most buried amino acid residues require nonpolar side chains, whereas few features of surface side chains are generally conserved. Other such phenotypically silent substitutions are described in Bowie, J. U. et al., supra, and the references cited therein. Typically seen as conservative substitutions are the replacements, one for another, among the aliphatic amino acids Ala, Val, Leu and Ile; interchange of the hydroxyl residues Ser and Thr, exchange of the acidic residues Asp and Glu, substitution between the amide residues Asn and Gln, exchange of the basic residues Lys and Arg and replacements among the aromatic residues Phe, Tyr.

Thus, the fragment, derivative or analog of the polypeptide of SEQ ID NO:2, or that encoded by the deposited cDNA, may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature or (extracellular domain, for LRF-2) polypeptide is fused with another compound, such as a compound to increase the half-life of the polypeptide (for example, polyethylene glycol), or (iv) one in which the additional amino acids are fused to the above form of the polypeptide, such as an IgG Fc fusion region peptide or leader or secretory sequence or a sequence which is employed for purification of the above form of the polypeptide or a proprotein sequence. Such fragments, derivatives and analogs are deemed to be within the scope of those skilled in the art from the teachings herein

Thus, the LRF-1 or LRF-2 polypeptide of the present invention may include one or more amino acid substitutions, deletions or additions, either from natural mutations or human manipulation. As indicated, changes are preferably of a minor nature, such as conservative amino acid substitutions that do not significantly affect the folding or activity of the protein (see Table 1).

TABLE 1
Conservative Amino Acid Substitutions.

| | |
|---|---|
| Aromatic | Phenylalanine |
| | Tryptophan |
| | Tyrosine |
| Hydrophobic | Leucine |
| | Isoleucine |
| | Valine |
| Polar | Glutamine |
| | Asparagine |
| Basic | Arginine |
| | Lysine |
| | Histidine |
| Acidic | Aspartic Acid |
| | Glutamic Acid |
| Small | Alanine |
| | Serine |
| | Threonine |
| | Methionine |
| | Glycine |

Amino acids in the LRF-1 or LRF-2 protein of the present invention that are essential for function can be identified by methods known in the art, such as site-directed mutagenesis or alanine-scanning mutagenesis (Cunningham and Wells, Science 244:1081-1085 (1989)). The latter procedure introduces single alanine mutations at every residue in the molecule. The resulting mutant molecules are then tested for biological activity such as receptor binding or in vitro or in vitro proliferative activity.

Of special interest are substitutions of charged amino acids with other charged or neutral amino acids which may produce proteins with highly desirable improved characteristics, such as less aggregation. Aggregation may not only reduce activity but also be problematic when preparing pharmaceutical formulations, because aggregates can be immunogenic (Pinckard et al., Clin. Exp. Immunol. 2:331-340 (1967); Robbins et al., Diabetes 36: 838-845 (1987); Cleland et al., Crit. Rev. Therapeutic Drug Carrier Systems 10:307-377 (1993).

Replacement of amino acids can also change the selectivity of the binding of a ligand to cell surface receptors. For example, Ostade et al., Nature 361:266-268 (1993) describes certain mutations resulting in selective binding of TNF-α to only one of the two known types of TNF receptors. Sites that are critical for ligand-receptor binding can also be determined by structural analysis such as crystallization, nuclear magnetic resonance or photoaffinity labeling (Smith et al, J. Mol. Biol. 224:899-904 (1992) and de Vos et al. Science 255:306-312 (1992)).

Since LRF-1 and LRF-2 are related to the NIF protein, as described above, to modulate rather than completely eliminate biological activities of LRF-1 or LRF-2 on leukocytes preferably mutations are made in sequences encoding amino acids in the conserved domains shared by NIF, LRF-1 and LRF-2 (see, for instance, FIGS. 3, 4, and 5), more preferably in residues within these regions which are not conserved in all of these sequences. Also forming part of the present invention are isolated polynucleotides comprising nucleic acid sequences which encode the above LRF-1 or LRF-2 mutants.

The polypeptides of the present invention are preferably provided in an isolated form, and preferably are substantially purified. A recombinantly produced version of the LRF-1 or LRF-2 polypeptide can be substantially purfied by the one-step method described in Smith and Johnson, Gene 67:31-40 (1988). Polypeptides of the invention also can be purified from natural or recombinant sources using anti-LRF-1 or LRF-2 antibodies of the invention in methods which are well known in the art of protein purification.

The invention further provides an isolated LRF-1 polypeptide comprising an amino acid sequence selected from the group consisting of: (a) the complete amino acid sequence of the full-length LRF-1 polypeptide sequence shown in SEQ ID NO:2 excepting the N-terminal methionine (i.e., positions −24 to +254 of SEQ ID NO:2); (b) the amino acid sequence of the predicted mature LRF-1 polypeptide shown at positions +1 to +254 in SEQ ID NO:2; (c) the complete amino acid sequence of the LRF-1 excepting the N-terminal methionine encoded by the cDNA clone contained in ATCC Deposit No 97860; and (d) the amino acid sequence of the mature LRF-l polypeptide encoded by the cDNA clone contained in ATCC Deposit No. 97860.

Also provided is an isolated LRF-2 polypeptide comprising an amino acid sequence selected from the group consisting of: (a) the complete amino acid sequence of the full-length LRF-2 polypeptide shown in SEQ ID NO:4 excepting the N-terminal methionine (i.e., positions −21 to +441 of SEQ ID NO:4); (b) the amino acid sequence of the predicted mature LRF-2 polypeptide shown at about position −2 to about position +441 or at about position +1 to about position +441 in SEQ ID NO:4; (c) the amino acid sequence of the predicted soluble mature LRF-2 shown at about position −2 to about position 418 or at about position +1 to about position +418 in SEQ ID NO:4; (d) the complete amino acid sequence of the full-length LRF-2 polypeptide excepting the N-terminal methionine encoded by the cDNA clone contained in ATCC Deposit No 97867; (e) the amino acid sequence of the mature LRF-2 polypeptide encoded by the cDNA clone contained in ATCC Deposit No. 97867; and (f) the amino acid sequence of the soluble mature LRF-2 polypeptide encoded by the cDNA clone contained in ATCC Deposit No. 97867 where the soluble form lacks the C-terminal sequence of about 23 amino acids of the mature LRF-2 polypeptide encoded by that cDNA.

Further polypeptides of the present invention include polypeptides which have at least 90% similarity, more preferably at least 95% similarity, and still more preferably at least 96%, 97%, 98% or 99% similarity to those described above. The polypeptides of the invention also comprise those which are at least 80% identical, more preferably at least 90% or 95% identical, still more preferably at least 96%, 97%, 98% or 99% identical to the polypeptide encoded by one of the deposited cDNAs or to the polypeptide of SEQ ID NO:2 or of SEQ ID NO:4, and also include portions of such polypeptides with at least 30 amino acids and more preferably at least 50 amino acids.

By “% similarity” for two polypeptides is intended a similarity score produced by comparing the amino acid sequences of the two polypeptides using the Bestfit program (Wisconsin Sequence Analysis Package, Version 8 for Unix, Genetics Computer Group, University Research Park, 575 Science Drive, Madison, Wis. 53711) and the default settings for determining similarity. Bestfit uses the local homology algorithm of Smith and Waterman (Advances in Applied Mathematics 2:482-489, 1981) to find the best segment of similarity between two sequences.

By a polypeptide having an amino acid sequence at least, for example, 95% “identical” to a reference amino acid sequence of a LRF-1 (or LRF-2) polypeptide is intended that the amino acid sequence of the polypeptide is identical to the reference sequence except that the polypeptide sequence may include up to five amino acid alterations per each 100 amino acids of the reference amino acid of the LRF-1 (or LRF-2) polypeptide. In other words, to obtain a polypeptide having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 90%, 95%, 96%, 97%, 98% or 99% identical to, for instance, the amino acid sequence shown in SEQ ID NO:2 or in SEQ ID NO:4, or to the amino acid sequence encoded by one of the deposited cDNA clones, can be determined conventionally using known computer programs such the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence according to the present invention, the parameters are set, of course, such that the percentage of identity is calculated over the full length of the reference amino acid sequence and that gaps in homology of up to 5% of the total number of amino acid residues in the reference sequence are allowed.

The polypeptide of the present invention could be used as a molecular weight marker on SDS-PAGE gels or on molecular sieve gel filtration columns using methods well known to those of skill in the art.

As described in detail below, the polypeptides of the present invention can also be used to raise polyclonal and monoclonal antibodies, which are useful in assays for detecting LRF-1 or LRF-2 protein expression as described below or as agonists and antagonists capable of enhancing or inhibiting LRF-1 of LRF-2 protein function. Further, such polypeptides can be used in the yeast two-hybrid system to “capture” LRF-1 of LRF-2 protein binding proteins which are also candidate agonists and antagonists according to the present invention. The yeast two hybrid system is described in Fields and Song, Nature 340:245-246 (1989).

Epitope-Bearing Portions

In another aspect, the invention provides a peptide or polypeptide comprising an epitope-bearing portion of a polypeptide of the invention. The epitope of this polypeptide portion is an immunogenic or antigenic epitope of a polypeptide of the invention. An “immunogenic epitope” is defined as a part of a protein that elicits an antibody response when the whole protein is the immunogen. On the other hand, a region of a protein molecule to which an antibody can bind is defined as an “antigenic epitope.” The number of immunogenic epitopes of a protein generally is less than the number of antigenic epitopes. See, for instance, Geysen et al., Proc. Natl. Acad. Sci. USA 81:3998-4002 (1983).

As to the selection of peptides or polypeptides bearing an antigenic epitope (i.e., that contain a region of a protein molecule to which an antibody can bind), it is well known in that art that relatively short synthetic peptides that mimic part of a protein sequence are routinely capable of eliciting an antiserum that reacts with the partially mimicked protein. See, for instance, Sutcliffe, J. G., Shinnick, T. M., Green, N. and Learner, R. A. (1983) “Antibodies that react with predetermined sites on proteins,” Science, 219:660-666. Peptides capable of eliciting protein-reactive sera are frequently represented in the primary sequence of a protein, can be characterized by a set of simple chemical rules, and are confined neither to immunodominant regions of intact proteins (i.e., immunogenic epitopes) nor to the amino or carboxyl terminals. Antigenic epitope-bearing peptides and polypeptides of the invention are therefore useful to raise antibodies, including monoclonal antibodies, that bind specifically to a polypeptide of the invention. See, for instance, Wilson el al., Cell 37:767-778 (1984) at 777.

Antigenic epitope-bearing peptides and polypeptides of the invention preferably contain a sequence of at least seven, more preferably at least nine and most preferably between about 15 to about 30 amino acids contained within the amino acid sequence of a polypeptide of the invention. Non-limiting examples of antigenic polypeptides or peptides that can be used to generate LRF-1-specific antibodies include: a polypeptide comprising amino acid residues from about His 19 to about Phe 45 in SEQ ID NO:2; a polypeptide comprising amino acid residues from about Ala 97 to about Ile 125 in SEQ ID NO:2; a polypeptide comprising amino acid residues from about Gly 154 to about Ile 195 in SEQ ID NO:2; and; a polypeptide comprising amino acid residues from about Leu 203 to about Leu 249 in SEQ ID NO:2. These polypeptide fragments have been determined to bear antigenic epitopes of the LRF-1 protein by the analysis of the Jameson-Wolf antigenic index, as shown in FIG. 6, above.

Non-limiting examples of antigenic polypeptides or peptides that can be used to generate LRF-2-specific antibodies include: a polypeptide comprising amino acid residues from about His 56 to about Asn 66 in SEQ ID NO:4; a polypeptide comprising amino acid residues from about Glu 82 to about Ser 94 in SEQ ID NO:4; a polypeptide comprising amino acid residues from about Glu 144 to about Pro 160 in SEQ ID NO:4; a polypeptide comprising amino acid residues from about Pro 294 to about Lys 318 in SEQ ID NO:4; a polypeptide comprising amino acid residues from about le 178 to about Thr 210 in SEQ ID NO:4; a polypeptide comprising amino acid residues from about Glu 239 to about Glu 257 in SEQ ID NO:4; a polypeptide comprising amino acid residues from about His 273 to about His 290 in SEQ ID NO:4; a polypeptide comprising amino acid residues from about Phe 352 to about Ala 363 in SEQ ID NO:4; a polypeptide comprising amino acid residues from about His 370 to about Thr 385 in SEQ ID NO:4; and a polypeptide comprising amino acid residues from about Ser 398 to about Ser 413 in SEQ ID NO:4. These polypeptide fragments have been determined to bear antigenic epitopes of the LRF-1 protein by the analysis of the Jameson-Wolf antigenic index, as shown in FIG. 7, above.

The epitope-bearing peptides and polypeptides of the invention may be produced by any conventional means. See, e.g., Houghten, R. A. (1985) “General method for the rapid solid-phase synthesis of large numbers of peptides: specificity of antigen-antibody interaction at the level of individual amino acids.” Proc. Natl. Acad. Sci. USA 82:5131-5135; this “Simultaneous Multiple Peptide Synthesis (SMPS)” process is further described in U.S. Pat. No. 4,631,211 to Houghten et al. (1986).

Epitope-bearing peptides and polypeptides of the invention are used to induce antibodies according to methods well known in the art. See, for instance, Sutcliffe et al., supra; Wilson et al., supra; Chow, M. et al., Proc. Natl. Acad. Sci. USA 82:910-914; and Bittle, F. J. et al., J. Gen. Virol. 66:2347-2354 (1985). Immunogenic epitope-bearing peptides of the invention, i.e., those parts of a protein that elicit an antibody response when the whole protein is the immunogen, are identified according to methods known in the art. See, for instance, Geysen et al., supra. Further still, U.S. Pat. No. 5,194,392 to Geysen (1990) describes a general method of detecting or determining the sequence of monomers (amino acids or other compounds) which is a topological equivalent of the epitope (i.e., a “mimotope”) which is complementary to a particular paratope (antigen binding site) of an antibody of interest. More generally, U.S. Pat. No. 4,433,092 to Geysen (1989) describes a method of detecting or determining a sequence of monomers which is a topographical equivalent of a ligand which is complementary to the ligand binding site of a particular receptor of interest. Similarly, U.S. Pat. No. 5,480,971 to Houghten, R. A. et al. (1996) on Peralkylated Oligopeptide Mixtures discloses linear C1-C7-alkyl peralkylated oligopeptides and sets and libraries of such peptides, as well as methods for using such oligopeptide sets and libraries for determining the sequence of a peralkylated oligopeptide that preferentially binds to an acceptor molecule of interest. Thus, non-peptide analogs of the epitope-bearing peptides of the invention also can be made routinely by these methods.

Fusion Proteins

As one of skill in the art will appreciate, LRF-1 and LRF-2 polypeptides of the present invention and the epitope-bearing fragments thereof described above can be combined with parts of the constant domain of immunoglobulins (IgG), resulting in chimeric polypeptides. These fusion proteins facilitate purification and show an increased half-life in vivo. This has been shown, e.g., for chimeric proteins consisting of the first two domains of the human CD4-polypeptide and various domains of the constant regions of the heavy or light chains of mammalian immunoglobulins (EP A 394,827; Traunecker et al., Nature 331:84-86 (1988)). Fusion proteins that have a disulfide-linked dimeric structure due to the IgG part can also be more efficient in binding and neutralizing other molecules than the monomeric LRF-1 protein or protein fragment alone (Fountoulakis et al., J. Biochem. 270:3958-3964 (1995)).

Antibodies

LRF-1-protein and LRF-2-protein specific antibodies for use in the present invention can be raised against the intact LRF-1 or LRF-2 proteins, respectively, or an antigenic polypeptide fragment thereof, which may be presented together with a carrier protein, such as an albumin, to an animal system (such as rabbit or mouse) or, if it is long enough (at least about 25 amino acids), without a carrier.

As used herein, the term “antibody” (Ab) or “monoclonal antibody” (Mab) is meant to include intact molecules as well as antibody fragments (such as, for example, Fab and F(ab′)2 fragments) which are capable of specifically binding to LRF-1 or LRF-2 protein. Fab and F(ab′)2 fragments lack the Fc fragment of intact antibody, clear more rapidly from the circulation, and may have less non-specific tissue binding of an intact antibody (Wahl et al., J. Nucl. Med. 24:316-325 (1983)). Thus, these fragments are preferred.

The antibodies of the present invention may be prepared by any of a variety of methods. For example, cells expressing the LRF-1 or LRF-2 protein or an antigenic fragment thereof can be administered to an animal in order to induce the production of sera containing polyclonal antibodies. In a preferred method, a preparation of LRF-1 or LRF-2 protein is prepared and purified to render it substantially free of natural contaminants. Such a preparation is then introduced into an animal in order to produce polyclonal antisera of greater specific activity.

In the most preferred method, the antibodies of the present invention are monoclonal antibodies (or LRF-1 or LRF-2 protein binding fragments thereof). Such monoclonal antibodies can be prepared using hybridoma technology (Köhler et al., Nature 256:495 (1975); Köhler et al., Eur. J. Immunol. 6:511 (1976); Köhler et al., Eur. J. Immunol. 6:292 (1976); Hammerling et al., in: Monoclonal Antibodies and T-Cell Hybridomas, Elsevier, N.Y., (1981) pp. 563-681). In general, such procedures involve immunizing an animal (preferably a mouse) with an LRF-1 or LRF-2 protein antigen or, more preferably, with a LRF-1 or LRF-2 protein-expressing cell. Suitable cells can be recognized by their capacity to bind anti-LRF-1 protein antibody. Such cells may be cultured in any suitable tissue culture medium; however, it is preferable to culture cells in Earle's modified Eagle's medium supplemented with 10% fetal bovine serum (inactivated at about 56° C.), and supplemented with about 10 g/l of nonessential amino acids, about 1,000 U/ml of penicillin, and about 100 μg/ml of streptomycin. The splenocytes of such mice are extracted and fused with a suitable myeloma cell line. Any suitable myeloma cell line may be employed in accordance with the present invention; however, it is preferable to employ the parent myeloma cell line (SP20), available from the American Type Culture Collection, Rockville, Md. After fusion, the resulting hybridoma cells are selectively maintained in HAT medium, and then cloned by limiting dilution as described by Wands et al. (Gastroenterology 80:225-232 (1981)). The hybridoma cells obtained through such a selection are then assayed to identify clones which secrete antibodies capable of binding the LRF-1 or LRF-2 protein antigen.

Alternatively, additional antibodies capable of binding to the LRF-1 protein antigen may be produced in a two-step procedure through the use of anti-idiotypic antibodies. Such a method makes use of the fact that antibodies are themselves antigens, and that, therefore, it is possible to obtain an antibody which binds to a second antibody. In accordance with this method, LRF-1 or LRF-2-protein specific antibodies are used to immunize an animal, preferably a mouse. The splenocytes of such an animal are then used to produce hybridoma cells, and the hybridoma cells are screened to identify clones which produce an antibody whose ability to bind to the LRF-1 or LRF-2-protein-specific antibody can be blocked by the LRF-1 or LRF-2 protein antigen. Such antibodies comprise anti-idiotypic antibodies to the LRF-1 of LRF-2 protein-specific antibody and can be used to immunize an animal to induce formation of further LRF-1 or LRF-2 protein-specific antibodies.

It will be appreciated that Fab and F(ab′)2 and other fragments of the antibodies of the present invention may be used according to the methods disclosed herein. Such fragments are typically produced by proteolytic cleavage, using enzymes such as papain (to produce Fab fragments) or pepsin (to produce F(ab′)2 fragments). Alternatively, LRF-1 or LRF-2 protein-binding fragments can be produced through the application of recombinant DNA technology or through synthetic chemistry.

For in vivo use of anti-LRF-1 or anti-LRF-2 in humans, it may be preferable to use “humanized” chimeric monoclonal antibodies. Such antibodies can be produced using genetic constructs derived from hybridoma cells producing the monoclonal antibodies described above. Methods for producing chimeric antibodies are known in the art. See, for review, Morrison, Science 229:1202 (1985); Oi et al., BioTechniques 4:214 (1986); Cabilly et al., U.S. Pat. No. 4,816,567; Taniguchi et al., EP 171496; Morrison et al., EP 173494; Neuberger et al., WO 8601533; Robinson et al., WO 8702671; Boulianne et al., Nature 312:643 (1984); Neuberger et al., Nature 314:268 (1985).

### Immune System-Related Other Disorders

Diagnosis

The present inventors have discovered that LRF-1 is expressed not only in human testes but also in dendritic cells (DC) which are the principal antigen presenting cells involved in primary immune responses; their major function is to obtain antigen in tissues, migrate to lymphoid organs, and activate T cells (Mohamadzadeh, M. et al., J. Immunol. 156: 3102-3106 (1996). For example, Langerhans cells (LC), which are skin-specific members of this family, have been shown to present a variety of antigens that may be generated in or penetrate into skin. In contact hypersensitivity, topical application of a reactive hapten activates LC to migrate out of the epidermis into draining lymph nodes, where they present this antigen to selected T cells. Human LC lines secrete relatively large amounts of various chemokines such as NAP-1/IL-8 and MIP-1α upon ligation of CD40 on cell surfaces. Thus, it is likely that LC possess the potential to produce a selected set of chemokines with chemotactic activities for T cells. DC are also the first immune cells to arrive at sites of inflammation on mucous membranes, the major site of sexual transmission of HIV. Weissman, D. et al., J. Immunol. 155:4111-4117 (1995).

It has further been discovered that a nucleotide sequence encoding the mature LRF-2 polypeptide having the amino acid sequence encoded by the cDNA clone contained in the host identified as ATCC Deposit No. 97867 is detectable by Northern blot not only in human fetal heart tissue where the deposited clone originated, but also in skeletal muscle and pancreas at much lower levels. Individual cDNA clones encoding all or part of the LRF-2 amino acid sequence (SEQ ID NO:4) also have been isolated from amygdala, fetal epithelium, striatum, microvascular endothelium, Jurkat T cells, breast, rhabdomyosarcoma, fetal bone, and smooth muscle.

For a number of disorders of the above tissues or cell s, significantly higher or lower levels of LRF-1 or LRF-2 gene expression may be detected in certain tissues (e.g., cancerous and wounded tissues) or bodily fluids (e.g., serum, plasma, urine, synovial fluid or spinal fluid) taken from an individual having such a disorder, relative to a “standard” LRF-1 or LRF-2 gene expression level, i.e., the expression level in healthy tissue from an individual not having the immune system disorder. Thus, the invention provides a diagnostic method useful during diagnosis of such a disorder, which involves: (a) assaying LRF-1 or LRF-2 gene expression level in cells or body fluid of an individual; (b) comparing the LRF-1 or LRF-2 gene expression level, respectively, with a standard LRF-1 or LRF-2 gene expression level, whereby an increase or decrease in the assayed gene expression level compared to the standard expression level is indicative of disorder in the pertinent system.

In particular, it is believed that certain tissues in mammals with cancer of cells in the immune system, particularly leukocytes, express significantly higher levels of the LRF-1 protein and mRNA encoding the LRF-1 protein when compared to a corresponding “standard” level. Further, it is believed that enhanced levels of the LRF-1 protein can be detected in certain body fluids (e.g., sera, plasma, urine, and spinal fluid) from mammals with such a cancer when compared to sera from mammals of the same species not having the cancer.

Similarly, it is believed that certain tissues in mammals with cancer of cells in the skeletal muscle and pancreas, as well as in the immune system (especially T cells), epithelium, striatum, microvascular endothelium, breast, bone, and smooth muscle express significantly higher levels of the LRF-2 protein and mRNA encoding the LRF-2 protein when compared to a corresponding “standard” level. Further, it is believed that enhanced levels of the LRF-2 protein can be detected in certain body fluids (e.g., sera, plasma, urine, and spinal fluid) from mammals with such a cancer when compared to sera from mammals of the same species not having the cancer.

In addition, the homology shared with the canine hookworm NIF polypeptide, as well as with the related plant pathogenesis-related (PR) proteins, indicates that the human LRF-1 and LRF-2 polypeptides also exhibit activities useful for modulation of immune system cell functions such as proliferation, differentiation, migration, adhesion and activation of leukocytes, particularly neutrophils, which ultimately permits modulation of defensive functions of these cells such as antimicrobial and anti-inflammatory activities.

The complete LRF-2 amino acid sequence (SEQ ID NO:4) also contains a peroxidase “signature” sequence (i.e., the amino acid sequence EVPSILAAHSL at positions 287-297 of FIGS. 2A, 2B, 2C, and 2D (positions 265-275 of SEQ ID NO:4). Peroxidases (EC 1.11.1.-) are heme-binding enzymes that carry out a variety of biosynthetic and degradative functions using hydrogen peroxide as the electron acceptor. Peroxidases are widely distributed throughout bacteria, fungi, plants, and vertebrates, including, for instance, the following: myeloperoxidase (EC 1.11.1.7) (MPO), which is found in granulocytes and monocytes and plays a major role in the oxygen-dependent microbicidal system of neutrophils; lactoperoxidase (EC 1.11.1.7) (LPO), which is a milk protein that acts as an antimicrobial agent; eosinophil peroxidase (EC 1.11.1.7) (EPO), an enzyme found in the cytoplasmic granules of eosinophils; and plant peroxidases (EC 1.11.1.7), some of which are expressed as a defense response toward wounding while others are involved in the metabolism of auxin and the biosynthesis of lignin. Since a major function of neutrophils involves release of toxic hydrogen peroxide, the peroxidase “signature” sequence in LRF-2 indicates that this particular protein is involved in carrying out biosynthetic and/or degradative functions (e.g., inflammatory and/or antimicrobial activities) using hydrogen peroxide released from neutrophils as the electron acceptor. In contrast the amino acid sequence of LRF-1 (FIGS. 1A and 1B and SEQ ID NO:2), while highly homologous with that of LRF-2 over the N-terminal region, terminates prior to the C-terminal region of LRF-2 containing the peroxidase signature sequence (see, FIGS. 2A, 2B, 2C, and 2D).

Based on the above expression patterns and homologies, it is believed that improper levels of LRF-1 or LRF-2 activities, due to defects in the level of expression or in the structure of the proteins, will lead to disturbances in immune system cell functions such as proliferation, differentiation, migration, adhesion and activation of leukocytes, particularly neutrophils, which ultimately will lead to deficiencies in defensive functions of these cells such as antimicrobial and anti-inflammatory activities. For instance, insufficient LRF-1 or LRF-2 activity can contribute to greater susceptibility to microbial infections (including viral, fungal, bacterial and parasite infections), allergy-related diseases, defective hematopoiesis, inflammatory diseases, defective wound healing and autoimmune diseases. More in particular, the present invention is useful for diagnosis or treatment of various immune system-related disorders in mammals, preferably humans. Such disorders include tumors, cancers, interstitial lung disease (such as Langerhans cell granulomatosis) and any disregulation of immune cell function including, but not limited to, autoimmunity, arthritis, leukemias, lymphomas, immunosuppression, immunity, humoral immunity, inflammatory bowel disease, myelosuppression, and the like. For LRF-2, deficiencies during development of the fetal heart, where the gene appears to be most highly expressed of all tissues examined to date, may lead to heart conditions in the newborn or adult, such as myocardosis or myocarditis.

Thus, the invention provides a diagnostic method useful during diagnosis of an immune system disorder, including cancers, which involves measuring the expression level of the gene encoding the LRF-1 or LRF-2 protein immune system tissue or other cells or body fluid from an individual and comparing the measured gene expression level with a standard LRF-1 or LRF-2 gene expression level, whereby an increase or decrease in the gene expression level compared to the standard is indicative of an immune system disorder. Where a diagnosis of a disorder in the immune system, including a malignancy, has already been made according to conventional methods, the present invention is useful as a prognostic indicator, whereby patients exhibiting enhance or depressed gene expression will experience a worse clinical outcome relative to patients expressing the LRF-1 or LRF-2 gene at a level nearer the standard level.

By “assaying the expression level of the gene encoding the LRF-1 (or LRF-2) protein” is intended qualitatively or quantitatively measuring or estimating the level of the LRF-1 protein or the level of the mRNA encoding the LRF-1 (or LRF-2) protein in a first biological sample either directly (e.g., by determining or estimating absolute protein level or mRNA level) or relatively (e.g., by comparing to the LRF-1 (or LRF-2) protein level or mRNA level in a second biological sample). Preferably, the LRF-1 (or LRF-2) protein level or mRNA level in the first biological sample is measured or estimated and compared to a standard LRF-1 (or LRF-2) protein level or mRNA level, the standard being taken from a second biological sample obtained from an individual not having the disorder or being determined by averaging levels from a population of individuals not having a disorder of the immune system. As will be appreciated in the art, once a standard LRF-1 (or LRF-2) protein level or mRNA level is known, it can be used repeatedly as a standard for comparison.

By “biological sample” is intended any biological sample obtained from an individual, body fluid, cell line, tissue culture, or other source which contains LRF-1 protein or mRNA. As indicated, biological samples include body fluids (such as sera, plasma, urine, synovial fluid and spinal fluid) which contain free mature or extracellular domains of LRF-1 or LRF-2 protein, immune system tissue, and other tissue sources found to express complete or mature or extracellular domain of the LRF-1 (or LRF-2) polypeptide or a receptor for LRF-1 (or LRF-2). Methods for obtaining tissue biopsies and body fluids from mammals are well known in the art. Where the biological sample is to include mRNA, a tissue biopsy is the preferred source.

Total cellular RNA can be isolated from a biological sample using any suitable technique such as the single-step guanidinium-thiocyanate-phenol-chloroform method described in Chomczynski and Sacchi, Anal. Biochem. 162:156-159 (1987). Levels of mRNA encoding the LRF-1 (or LRF-2) protein are then assayed using any appropriate method. These include Northern blot analysis, S1 nuclease mapping, the polymerase chain reaction (PCR), reverse transcription in combination with the polymerase chain reaction (RT-PCR), and reverse transcription in combination with the ligase chain reaction (RT-LCR).

Assaying LRF-1 (or LRF-2) protein levels in a biological sample can occur using antibody-based techniques. For example, LRF-1 (or LRF-2) protein expression in tissues can be studied with classical immunohistological methods (Jalkanen, M., et al., J. Cell. Biol. 101.976-985 (1985); Jalkanen, M., et al., J. Cell Biol. 105:3087-3096 (1987)). Other antibody-based methods useful for detecting LRF-1 (or LRF-2) protein gene expression include immunoassays, such as the enzyme linked immunosorbent assay (ELISA) and the radioimmunoassay (RIA). Suitable antibody assay labels are known in the art and include enzyme labels, such as, glucose oxidase, and radioisotopes, such as iodine (¹²⁵I, ¹²¹I), carbon (¹⁴C), sulfur (³⁵S), tritium (³H), indium (¹¹²In), and technetium (^{99m}Tc), and fluorescent labels, such as fluorescein and rhodamine, and biotin.

In addition to assaying LRF-1 (or LRF-2) protein levels in a biological sample obtained from an individual, LRF-1 (or LRF-2) protein can also be detected in vivo by imaging. Antibody labels or markers for in vivo imaging of LRF-1 (or LRF-2) protein include those detectable by X-radiography, NMR or ESR. For X-radiography, suitable labels include radioisotopes such as barium or cesium, which emit detectable radiation but are not overtly harmful to the subject. Suitable markers for NMR and ESR include those with a detectable characteristic spin, such as deuterium, which may be incorporated into the antibody by labeling of nutrients for the relevant hybridoma.

An LRF-1 (or LRF-2) protein-specific antibody or antibody fragment which has been labeled with an appropriate detectable imaging moiety, such as a radioisotope (for example, ¹³¹I, ¹¹²In, ^{99m}Tc), a radio-opaque substance, or a material detectable by nuclear magnetic resonance, is introduced (for example, parenterally, subcutaneously or intraperitoneally) into the mammal to be examined for immune system disorder. It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ^{99m}Tc. The labeled antibody or antibody fragment will then preferentially accumulate at the location of cells which contain LRF-1 (or LRF-2) protein. In vivo tumor imaging is described in S. W. Burchiel et al., “Immunopharmacokinetics of Radiolabeled Antibodies and Their Fragments” (Chapter 13 in Tumor Imaging: The Radiochemical Detection of Cancer, S. W. Burchiel and B. A. Rhodes, eds., Masson Publishing Inc. (1982)). Treatment

As noted above, LRF-1 and LRF-2 polynucleotides and polypeptides are useful for diagnosis of conditions involving abnormally high or low expression of LRF-1 or LRF-2 activities. Given the cells and tissues where LRF-1 (or LRF-2) is expressed, as well as the activities modulated by LRF-1 (or LRF-2), it is readily apparent that a substantially altered (increased or decreased) level of expression of LRF-1 (or LRF-2) in an individual compared to the standard or “normal” level produces pathological conditions related to the bodily system(s) in which LRF-1 (or LRF-2) is expressed and/or is active.

It will also be appreciated by one of ordinary skill that, the mature LRF-1 (or LRF-2) protein of the invention is released in soluble form from the cells which express the LRF-1 (or LRF-2), by proteolytic cleavage. In addition, a soluble mature (extracellular domain) form of LRF-2, which in one form is a type 1 integral membrane protein, may be released from cells expressing LRF-2, by further proteolytic cleavage. Therefore, when mature LRF-1 or soluble extracellular domain of LRF-2 is added from an exogenous source to cells, tissues or the body of an individual, the protein will exert its physiological activities on its target cells of that individual. Also, cells expressing the type 1 integral membrane form of LRF-2 protein may be added to cells, tissues or the body of an individual, and these added cells will bind to cells expressing receptor for LRF-2, whereby the cells expressing LRF-2 can cause actions (e.g. stimulation) on the receptor-bearing target cells.

Therefore, it will be appreciated that conditions caused by a decrease in the standard or normal level of LRF-1 (or LRF-2) activity in an individual, particularly disorders of the immune system, can be treated by administration of LRF-1 (or LRF-2) polypeptide in the form mature protein or, for LRF-2, soluble extracellular domain or cells expressing the complete protein). Thus, the invention also provides a method of treatment of an individual in need of an increased level of LRF-1 (or LRF-2) activity comprising administering to such an individual a pharmaceutical composition comprising an amount of an isolated LRF-1 (or LRF-2) polypeptide (or LRF-2-expressing cells) of the invention, particularly a mature form of the LRF-1 protein or a soluble extracellular form of the LRF-2 protein of the invention, effective to increase the LRF-1 (or LRF-2) activity level in such an individual.

Those of skill in the art will recognize other indications, which may not involved improper expression of LRF-1 or LRF-2 activities, but which nevertheless would benefit from administration of LRF-1 or LRF-2 polypeptides of the invention. Thus, LRF-1 or LRF-2 polypeptides may also be employed to enhance host defenses against resistant chronic and acute infections, for example, mycobacterial infections via the attraction and activation of microbiocidal leukocytes. LRF-1 (or LRF-2) may also be employed for the treatment of auto-immune diseases (e.g., T-cell mediated conditions), allergic diseases, inflammatory diseases., and to stimulate wound healing. LRF-1 (or LRF-2) may also be employed to regulate hematopoiesis, by regulating the activation and differentiation of various hematopoietic progenitor cells, for example, to release mature leukocytes from the bone marrow following chemotherapy, i.e., in stem cell mobilization. LRF-2 may also be used to treat myocardosis and myocarditis.

Formulations

The LRF-1 (or LRF-2) polypeptide composition will be formulated and dosed in a fashion consistent with good medical practice, taking into account the clinical condition of the individual patient (especially the side effects of treatment with LRF-1 1 (or LRF-2) polypeptide alone), the site of delivery of the LRF-1 1 (or LRF-2) polypeptide composition, the method of administration, the scheduling of administration, and other factors known to practitioners. The “effective amount” of LRF-1 1 (or LRF-2) polypeptide for purposes herein is thus determined by such considerations.

As a general proposition, the total pharmaceutically effective amount of LRF-1 1 (or LRF-2) polypeptide administered parenterally per dose will be in the range of about 1 μg/kg/day to 10 mg/kg/day of patient body weight, although, as noted above, this will be subject to therapeutic discretion. More preferably, this dose is at least 0.01 mg/kg/day, and most preferably for humans between about 0.01 and 1 mg/kg/day for the hormone. If given continuously, the LRF-1 (or LRF-2) polypeptide is typically administered at a dose rate of about 1 μg/kg/hour to about 50 μg/kg/hour, either by 1-4 injections per day or by continuous subcutaneous infusions, for example, using a mini-pump. An intravenous bag solution may also be employed. The length of treatment needed to observe changes and the interval following treatment for responses to occur appears to vary depending on the desired effect.

Pharmaceutical compositions containing the LRF-1 1 (or LRF-2) polypeptide of the invention may be administered orally, rectally, parenterally, intracistemally, intravaginally, intraperitoneally, topically (as by powders, ointments, drops or transdermal patch), bucally, or as an oral or nasal spray. By “pharmaceutically acceptable carrier” is meant a non-toxic solid, semisolid or liquid filler, diluent, encapsulating material or formulation auxiliary of any type. The term “parenteral” as used herein refers to modes of administration which include intravenous, intramuscular, intraperitoneal, intrasternal, subcutaneous and intraarticular injection and infusion.

The LRF-1 1 (or LRF-2) polypeptide is also suitably administered by sustained-release systems. Suitable examples of sustained-release compositions include semi-permeable polymer matrices in the form of shaped articles, e.g., films, or mirocapsules. Sustained-release matrices include polylactides (U.S. Pat. No. 3,773,919, EP 58,481), copolymers of L-glutamic acid and gamma-ethyl-L-glutamate (Sidman, U. et al., Biopolymers 22:547-556 (1983)), poly (2-hydroxyethyl methacrylate) (R. Langer et al., J. Biomed. Mater. Res. 15:167-277 (1981), and R. Langer, Chem. Tech. 12:98-105 (1982)), ethylene vinyl acetate (R. Langer et al., Id.) or poly-D-(−)-3-hydroxybutyric acid (EP 133,988). Sustained-release LRF-1 1(or LRF-2) polypeptide compositions also include liposomally entrapped LRF-l 1 (or LRF-2) polypeptide. Liposomes containing LRF-1 1 (or LRF-2) polypeptide are prepared by methods known per se: DE 3,218,121; Epstein et al., Proc. Natl. Acad. Sci. (USA) 82:3688-3692 (1985); Hwang et al., Proc. Natl. Acad. Sci. (USA) 77:4030-4034 (1980); EP 52,322; EP 36,676; EP 88,046; EP 143,949; EP 142,641; Japanese Pat. Appl. 83-118008; U.S. Pat. Nos. 4,485,045 and 4,544,545; and EP 102,324. Ordinarily, the liposomes are of the small (about 200-800 Angstroms) unilamellar type in which the lipid content is greater than about 30 mol. percent cholesterol, the selected proportion being adjusted for the optimal LRF-1 1 (or LRF-2) polypeptide therapy.

For parenteral administration, in one embodiment, the LRF-1 1 (or LRF-2) polypeptide is formulated generally by mixing it at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation. For example, the formulation preferably does not include oxidizing agents and other compounds that are known to be deleterious to polypeptides.

Generally, the formulations are prepared by contacting the LRF-1 1 (or LRF-2) polypeptide uniformly and intimately with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringer's solution, and dextrose solution. Non-aqueous vehicles such as fixed oils and ethyl oleate are also useful herein, as well as liposomes.

The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, manose, or dextrins; chelating agents such as EDTA; sugar alcohols such as mannitol or sorbitol; counterions such as sodium; and/or nonionic surfactants such as polysorbates, poloxamers, or PEG.

The LRF-1 1 (or LRF-2) polypeptide is typically formulated in such vehicles at a concentration of about 0.1 mg/ml to 100 mg/ml, preferably 1-10 mg/ml, at a pH of about 3 to 8. It will be understood that the use of certain of the foregoing excipients, carriers, or stabilizers will result in the formation of LRF-1 1 (or LRF-2) polypeptide salts. 101621 LRF-1 1 (or LRF-2) polypeptide to be used for therapeutic administration must be sterile. Sterility is readily accomplished by filtration through sterile filtration membranes (e.g., 0.2 micron membranes). Therapeutic LRF-1 (or LRF-2) polypeptide compositions generally are placed into a container having a sterile access port, for example, an intravenous solution bag or vial having a stopper pierceable by a hypodermic injection needle.

LRF-1 (or LRF-2) polypeptide ordinarily will be stored in unit or multi-dose containers, for example, sealed ampoules or vials, as an aqueous solution or as a lyophilized formulation for reconstitution. As an example of a lyophilized formulation, 10-ml vials are filled with 5 ml of sterile-filtered 1% (w/v) aqueous LRF-1 polypeptide solution, and the resulting mixture is lyophilized. The infusion solution is prepared by reconstituting the lyophilized LRF-1 (or LRF-2) polypeptide using bacteriostatic Water-for-Injection.

The invention also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration. In addition, the polypeptides of the present invention may be employed in conjunction with other therapeutic compounds.

Agonists and Antagonists—Assays and Molecules

The invention also provides a method of screening compounds to identify those which enhance or block the action of LRF-1 (or LRF-2) on cells, such as its interaction with LRF-1-binding molecules such as receptor molecules. An agonist is a compound which increases the natural biological functions of LRF-1 (or LRF-2) or which functions in a manner similar to LRF-1 (or LRF-2), while antagonists decrease or eliminate such functions.

In another aspect of this embodiment the invention provides a method for identifying a receptor protein or other ligand-binding protein which binds specifically to a LRF-1 (or LRF-2) polypeptide. For example, a cellular compartment, such as a membrane or a preparation thereof, may be prepared from a cell that expresses a molecule that binds LRF-1 (or LRF-2). The preparation is incubated with labeled LRF-1 (or LRF-2). LRF-1 (or LRF-2) and complexes of LRF-1 (or LRF-2) bound to the receptor or other binding protein are isolated and characterized according to routine methods known in the art. Alternatively, the LRF-1 (or LRF-2) polypeptide may be bound to a solid support so that binding molecules solubilized from cells are bound to the column and then eluted and characterized according to routine methods.

In the assay of the invention for agonists or antagonists, a cellular compartment, such as a membrane or a preparation thereof, may be prepared from a cell that expresses a molecule that binds LRF-1 (or LRF-2), such as a molecule of a signaling or regulatory pathway modulated by LRF-1 (or LRF-2). The preparation is incubated with labeled LRF-1 (or LRF-2) in the absence or the presence of a candidate molecule which may be a LRF-1 (or LRF-2) agonist or antagonist. The ability of the candidate molecule to bind the binding molecule is reflected in decreased binding of the labeled ligand. Molecules which bind gratuitously, i.e., without inducing the effects of LRF-1 (or LRF-2) on binding the LRF-1 (or LRF-2) binding molecule, are most likely to be good antagonists. Molecules that bind well and elicit effects that are the same as or closely related to LRF-1 (or LRF-2) are agonists.

LRF-1-(or LRF-2-) like effects of potential agonists and antagonists may by measured, for instance, by determining activity of a second messenger system following interaction of the candidate molecule with a cell or appropriate cell preparation, and comparing the effect with that of LRF-1 (or LRF-2) or molecules that elicit the same effects as LRF-1 (or LRF-2). Second messenger systems that may be useful in this regard include but are not limited to AMP guanylate cyclase, ion channel or phosphoinositide hydrolysis second messenger systems.

Another example of an assay for LRF-1 (or LRF-2) antagonists is a competitive assay that combines LRF-1 (or LRF-2) and a potential antagonist with membrane-bound LRF-1 (or LRF-2) receptor molecules or recombinant LRF-1 receptor molecules under appropriate conditions for a competitive inhibition assay. LRF-1 (or LRF-2) can be labeled, such as by radioactivity, such that the number of LRF-1 (or LRF-2) molecules bound to a receptor molecule can be determined accurately to assess the effectiveness of the potential antagonist.

Potential antagonists include small organic molecules, peptides, polypeptides and antibodies that bind to a polypeptide of the invention and thereby inhibit or extinguish its activity. Potential antagonists also may be small organic molecules, a peptide, a polypeptide such as a closely related protein or antibody that binds the same sites on a binding molecule, such as a receptor molecule, without inducing LRF-1-(or LRF-2-) induced activities, thereby preventing the action of LRF-1 (or LRF-2) by excluding LRF-1 (or LRF-2) from binding.

Other potential antagonists include antisense molecules. Antisense technology can be used to control gene expression through antisense DNA or RNA or through triple-helix formation. Antisense techniques are discussed, for example, in Okano, J. Neurochem. 56: 560 (1991); “Oligodeoxynucleotides as Antisense Inhibitors of Gene Expression.” CRC Press, Boca Raton, Fla. (1988). Triple helix formation is discussed in, for instance Lee et al., Nucleic Acids Research 6: 3073 (1979); Cooney et al., Science 241: 456 (1988); and Dervan et al., Science 251: 1360 (1991). The methods are based on binding of a polynucleotide to a complementary DNA or RNA. For example, the 5′ coding portion of a polynucleotide that encodes the mature polypeptide of the present invention may be used to design an antisense RNA oligonucleotide of from about 10 to 40 base pairs in length. A DNA oligonucleotide is designed to be complementary to a region of the gene involved in transcription thereby preventing transcription and the production of LRF-1. The antisense RNA oligonucleotide hybridizes to the mRNA in vivo and blocks translation of the mRNA molecule into LRF-1 (or LRF-2) polypeptide. The oligonucleotides described above can also be delivered to cells such that the antisense RNA or DNA may be expressed in vivo to inhibit production of LRF-1 (or LRF-2) protein.

The agonists and antagonists may be employed in a composition with a pharmaceutically acceptable carrier, e.g., as described above. The agonists may be employed, for instance, to treat conditions resulting from insufficient expression of an LRF-1 or LRF-2 activity. The antagonists may be employed, for instance, to treat conditions resulting from excessive expression of an LRF-1 or LRF-2 activity, as described above.

Gene Mapping

The nucleic acid molecules of the present invention are also valuable for chromosome identification. The sequence is specifically targeted to and can hybridize with a particular location on an individual human chromosome. Moreover, there is a current need for identifying particular sites on the chromosome. Few chromosome marking reagents based on actual sequence data (repeat polymorphisms) are presently available for marking chromosomal location. The mapping of DNAs to chromosomes according to the present invention is an important first step in correlating those sequences with genes associated with disease.

In certain preferred embodiments in this regard, the cDNA herein disclosed is used to clone genomic DNA of a LRF-1 (or LRF-2) protein gene. This can be accomplished using a variety of well known techniques and libraries, which generally are available commercially. The genomic DNA then is used for in situ chromosome mapping using well known techniques for this purpose.

In addition, in some cases, sequences can be mapped to chromosomes by preparing PCR primers (preferably 15-25 bp) from the cDNA. Computer analysis of the 3′ untranslated region of the gene is used to rapidly select primers that do not span more than one exon in the genomic DNA, thus complicating the amplification process. These primers are then used for PCR screening of somatic cell hybrids containing individual human chromosomes. Fluorescence in situ hybridization (“FISH”) of a cDNA clone to a metaphase chromosomal spread can be used to provide a precise chromosomal location in one step. This technique can be used with probes from the cDNA as short as 50 or 60 bp. For a review of this technique, see Verma et al., Human Chromosomes: A Manual Of Basic Techniques, Pergamon Press, New York (1988).

Once a sequence has been mapped to a precise chromosomal location, the physical position of the sequence on the chromosome can be correlated with genetic map data. Such data are found, for example, in V. McKusick, Mendelian Inheritance In Man, available on-line through Johns Hopkins University, Welch Medical Library. The relationship between genes and diseases that have been mapped to the same chromosomal region are then identified through linkage analysis (coinheritance of physically adjacent genes).

Next, it is necessary to determine the differences in the cDNA or genomic sequence between affected and unaffected individuals. If a mutation is observed in some or all of the affected individuals but not in any normal individuals, then the mutation is likely to be the causative agent of the disease.

Having generally described the invention, the same will be more readily understood by reference to the following examples, which are provided by way of illustration and are not intended as limiting.

### EXAMPLES

### Example 1

### Expression and Purification of LRF-1 and LRF-2 in E. coli

The bacterial expression vector pQE60 is used for bacterial expression in this example (QIAGEN, Inc., 9259 Eton Avenue, Chatsworth, Calif., 91311). pQE60 encodes ampicillin antibiotic resistance (“Ampr”) and contains a bacterial origin of replication (“ori”), an IPTG inducible promoter, a ribosome binding site (“RBS”), six codons encoding histidine residues that allow affinity purification using nickel-nitrilo-tri-acetic acid (“Ni-NTA”) affinity resin sold by QIAGEN, Inc., supra, and suitable single restriction enzyme cleavage sites. These elements are arranged such that a DNA fragment encoding a polypeptide may be inserted in such as way as to produce that polypeptide with the six His residues (i.e., a “6×His tag”) covalently linked to the carboxyl terminus of that polypeptide. However, in this example, the polypeptide coding sequence is inserted such that translation of the six His codons is prevented and, therefore, the polypeptide is produced with no 6×His tag.

The DNA sequence encoding the desired portion of the LRF-1 protein comprising the mature of the LRF-1 amino acid sequence is amplified from the deposited cDNA clone using PCR oligonucleotide primers which anneal to the amino terminal sequences of the desired portion of the LRF-1 protein and to sequences in the deposited construct 3′ to the cDNA coding sequence. Additional nucleotides containing restriction sites to facilitate cloning in the pQE60 vector are added to the 5′ and 3′ sequences, respectively.

For cloning the mature form of the LRF-1 protein, the 5′ primer has the sequence 5′ CG CCC ATG GCC AGA TTT TTG CCA GA 3′ (SEQ ID NO: 12) containing the underlined NcoI restriction site followed by 15 nucleotides of the amino terminal coding sequence of the mature LRF-1 sequence in SEQ ID NO: 1. One of ordinary skill in the art would appreciate, of course, that the point in the protein coding sequence where the 5′ primer begins may be varied to amplify a DNA segment encoding any desired portion of the complete LRF-1 protein shorter or longer than the mature form of the protein. The 3′ primer has the sequence 5′ CGC AAG CTT GAA TGT GGC ACA GTG 3′ (SEQ ID NO: 13) containing the underlined HindIII restriction site followed by 15 nucleotides complementary to the 3′ end of the coding sequence of the LRF-1 DNA sequence in FIGS. 1A and 1B (SEQ ID NO: 1).

The amplified LRF-1 DNA fragment and the vector pQE60 are digested with NcoI and HindIII and the digested DNAs are then ligated together. Insertion of the LRF-1 DNA into the restricted pQE60 vector places the LRF-1 protein coding region including its associated stop codon downstream from the IPTG-inducible promoter and in-frame with an initiating AUG. The associated stop codon prevents translation of the six histidine codons downstream of the insertion point.

For cloning the soluble extracellular form of the LRF-2 protein, described above, the 5′ primer has the sequence 5′ CGC GGA TCC GGC CCC GTT GGA GCC CTC 3′ (SEQ ID NO:14) containing the underlined BamHI restriction site followed by 18 nucleotides of the amino terminal coding sequence of the mature LRF-2 sequence in SEQ ID NO:3. One of ordinary skill in the art would appreciate, of course, that the point in the protein coding sequence where the 5′ primer begins may be varied to amplify a DNA segment encoding any desired portion of the complete LRF-2 protein shorter or longer than the mature form of the protein. The 3′ primer has the sequence 5′ CGG GGT ACC AAG CTT TCA GCC CCA CAC ATG ACC 3′ (SEQ ID NO: 15) containing the underlined HindIII restriction site followed by 18 nucleotides complementary to a sequence in the 3′ end of the coding sequence of the LRF-2 DNA sequence in FIGS. 2A, 2B, 2C, and 2D, which is upstream of the indicated transmembrane domain so that this domain is not amplified and included in the expression vector.

The amplified LRF-2 DNA fragment and the vector pQE60 are digested with BamHI and HindIII and the digested DNAs are then ligated together. Insertion of the LRF-2 DNA into the restricted pQE60 vector places the LRF-2 protein coding region including its associated stop codon downstream from the IPTG-inducible promoter and in-frame with an initiating AUG. The associated stop codon prevents translation of the six histidine codons downstream of the insertion point.

For either the LRF-1 or LRF-2 constructs, the ligation mixture is transformed into competent E. coli cells using standard procedures such as those described in Sambrook et al., Molecular Cloning: a Laboratory Manual, 2nd Ed.; Cold Spring Harbor Laboratory Press, Cold Spring Harbor, N.Y. (1989). E. coli strain M15/rep4, containing multiple copies of the plasmid pREP4, which expresses the lac repressor and confers kanamycin resistance (“Kanr”), is used in carrying out the illustrative example described herein. This strain, which is only one of many that are suitable for expressing LRF-1 of LRF-2 protein, is available commercially from QIAGEN, Inc., supra. Transformants are identified by their ability to grow on LB plates in the presence of ampicillin and kanamycin. Plasmid DNA is isolated from resistant colonies and the identity of the cloned DNA confirmed by restriction analysis, PCR and DNA sequencing.

Clones containing the desired constructs are grown overnight (“O/N”) in liquid culture in LB media supplemented with both ampicillin (100 μg/ml) and kanamycin (25 μg/ml). The O/N culture is used to inoculate a large culture, at a dilution of approximately 1:25 to 1:250. The cells are grown to an optical density at 600 nm (“OD600”) of between 0.4 and 0.6. isopropyl-b-D-thiogalactopyranoside (“IPTG”) is then added to a final concentration of 1 mM to induce transcription from the lac repressor sensitive promoter, by inactivating the lacI repressor. Cells subsequently are incubated further for 3 to 4 hours. Cells then are harvested by centrifugation.

To purify the LRF-1 or LRF-2 polypeptide, the cells are then stirred for 3-4 hours at 4° C. in 6M guanidine-HCl, pH 8. The cell debris is removed by centrifugation, and the supernatant containing the LRF-1 of LRF-2 is dialyzed against 50 mM Na-acetate buffer pH 6, supplemented with 200 mM NaCl. Alternatively, the protein can be successfully refolded by dialyzing it against 500 mM NaCl, 20% glycerol, 25 mM Tris/HCl pH 7.4, containing protease inhibitors. After renaturation the protein can be purified by ion exchange, hydrophobic interaction and size exclusion chromatography. Alternatively, an affinity chromatography step such as an antibody column can be used to obtain pure LRF-1 LRF-2 protein. The purified protein is stored at 4° C. or frozen at −80° C.

The following alternative method may be used to purify LRF-1 expressed in E. coli when it is present in the form of inclusion bodies. Unless otherwise specified, all of the following steps are conducted at 4-10° C.

Upon completion of the production phase of the E. Coli fermentation, the cell culture is cooled to 4-10° C. and the cells are harvested by continuous centrifugation at 15,000 rpm (Heraeus Sepatech). On the basis of the expected yield of protein per unit weight of cell paste and the amount of purified protein required, an appropriate amount of cell paste, by weight, is suspended in a buffer solution containing 100 mM Tris, 50 mM EDTA, pH 7.4. The cells are dispersed to a homogeneous suspension using a high shear mixer.

The cells are then lysed by passing the solution through a microfluidizer (Microfuidics, Corp. or APV Gaulin, Inc.) twice at 4000-6000 psi. The homogenate is then mixed with NaCl solution to a final concentration of 0.5 M NaCl, followed by centrifugation at 7000×g for 15 min. The resultant pellet is washed again using 0.5M NaCl, 100 mM Tris, 50 mM EDTA, pH 7.4.

The resulting washed inclusion bodies are solubilized with 1.5 M guanidine hydrochloride (GuHCl) for 2-4 hours. After 7000×g centrifugation for 15 min., the pellet is discarded and the LRF-1 or LRF-2 polypeptide-containing supernatant is incubated at 4° C. overnight to allow further GuHCl extraction.

Following high speed centrifugation (30,000×g) to remove insoluble particles, the GuHCl solubilized protein is refolded by quickly mixing the GuHCl extract with 20 volumes of buffer containing 50 mM sodium, pH 4.5, 150 mM NaCl, 2 mM EDTA by vigorous stirring. The refolded diluted protein solution is kept at 4° C. without mixing for 12 hours prior to further purification steps.

To clarify the refolded LRF-1 or LRF-2 polypeptide solution, a previously prepared tangential filtration unit equipped with 0.16 μm membrane filter with appropriate surface area (e.g., Filtron), equilibrated with 40 mM sodium acetate, pH 6.0 is employed. The filtered sample is loaded onto a cation exchange resin (e.g., Poros HS-50, Perseptive Biosystems). The column is washed with 40 mM sodium acetate, pH 6.0 and eluted with 250 mM, 500 mM, 1000 mM, and 1500 mM NaCl in the same buffer, in a stepwise manner. The absorbance at 280 mm of the effluent is continuously monitored. Fractions are collected and further analyzed by SDS-PAGE.

Fractions containing the LRF-1 or LRF-2 polypeptide are then pooled and mixed with 4 volumes of water. The diluted sample is then loaded onto a previously prepared set of tandem columns of strong anion (Poros HQ-50, Perseptive Biosystems) and weak anion (Poros CM-20, Perseptive Biosystems) exchange resins. The columns are equilibrated with 40 mM sodium acetate, pH 6.0. Both columns are washed with 40 mM sodium acetate, pH 6.0, 200 mM NaCl. The CM-20 column is then eluted using a 10 column volume linear gradient ranging from 0.2 M NaCl, 50 mM sodium acetate, pH 6.0 to 1.0 M NaCl, 50 mM sodium acetate, pH 6.5. Fractions are collected under constant A₂₈₀ monitoring of the effluent. Fractions containing the LRF-1 or LRF-2 polypeptide (determined, for instance, by 16% SDS-PAGE) are then pooled.

The resultant LRF-1 or LRF-2 polypeptide exhibits greater than 95% purity after the above refolding and purification steps. No major contaminant bands are observed from Commassie blue stained 16% SDS-PAGE gel when 5 μg of purified protein is loaded. The purified protein is also tested for endotoxin/LPS contamination, and typically the LPS content is less than 0.1 ng/ml according to LAL assays.

### Example 2

### Cloning and Expression of LRF-1 and LRF-2 Protein in a Baculovirus Expression System

In this illustrative example, the plasmid shuttle vector pA2 is used to insert the cloned DNA encoding complete protein, including its naturally associated secretory signal (leader) sequence, into a baculovirus to express the mature LRF-1 protein or the soluble extracellular domain of the LRF-2 protein, using standard methods as described in Summers et al., A Manual of Methods for Baculovirus Vectors and Insect Cell Culture Procedures, Texas Agricultural Experimental Station Bulletin No. 1555 (1987). This expression vector contains the strong polyhedrin promoter of the Autographa californica nuclear polyhedrosis virus (AcMNPV) followed by convenient restriction sites such as BamHI, Xba I and Asp718. The polyadenylation site of the simian virus 40 (“SV40”) is used for efficient polyadenylation. For easy selection of recombinant virus, the plasmid contains the beta-galactosidase gene from E. coli under control of a weak Drosophila promoter in the same orientation, followed by the polyadenylation signal of the polyhedrin gene. The inserted genes are flanked on-both sides by viral sequences for cell-mediated homologous recombination with wild-type viral DNA to generate a viable virus that express the cloned polynucleotide.

Many other baculovirus vectors could be used in place of the vector above, such as pAc373, pVL941 and pAcIM1, as one skilled in the art would readily appreciate, as long as the construct provides appropriately located signals for transcription, translation, secretion and the like, including a signal peptide and an in-frame AUG as required. Such vectors are described, for instance, in Luckow et al., Virology 170:31-39 (1989).

The cDNA sequence encoding the full length LRF-l protein in the deposited clone, including the AUG initiation codon and the naturally associated leader sequence shown in SEQ ID NO:2, is amplified using PCR oligonucleotide primers corresponding to the 5′ and 3′ sequences of the gene. The 5′ primer has the sequence 5′ CGC GGA TCC GCC ATC ATG GCA GTA GGG GGC GTT TTG 3′ (SEQ ID NO: 16) containing the underlined BamHI restriction enzyme site, an efficient signal for initiation of translation in eukaryotic cells, as described by Kozak, M., J. Mol. Biol. 196:947-950 (1987), followed by 18 nucleotides of the sequence of the complete LRF-1 protein shown in FIGS. 1A and 1B, beginning with the AUG initiation codon. The 3′ primer has the sequence 5′ CGC GGT ACC GAA TGT GGC ACA GTG 3′ (SEQ ID NO: 17) containing the underlined Asp718 restriction site followed by 15 nucleotides complementary to the 3′ noncoding sequence in FIGS. 1A and 1B (SEQ ID NO:1).

Similarly, for expression of the integral membrane protein form of LRF-2, the cDNA sequence encoding the complete LRF-2 protein in the deposited clone, including the AUG initiation codon and the naturally associated leader sequence shown in SEQ ID NO:4, is amplified using PCR oligonucleotide primers corresponding to the 5′ and 3′ sequences of the gene. The 5′ primer has the sequence 5′ CGC GGA TCC GCC ATC ATG CAC GGC TCC TGC AG 3′ (SEQ ID NO: 18) containing the underlined BamHI restriction enzyme site, an efficient signal for initiation of translation in eukaryotic cells, as described by Kozak, M., J. Mol. Biol. 196:947-950 (1987), followed by 17 nucleotides of the sequence of the complete LRF-2 protein shown in FIGS. 2A, 2B, 2C, and 2D, beginning with the AUG initiation codon. The 3′ primer has the sequence 5′ CGC GGT ACC GTC TCT CAC TTG GAG GA 3′ (SEQ ID NO: 19) containing the underlined Asp718 restriction site followed by 17 nucleotides complementary to the 3′ noncoding sequence in FIGS. 2A, 2B, 2C, and 2D. For expression of the soluble extracellular domain of the LRF-2 polypeptide, without the transmembrane domain, the extracellular portion of the cDNA sequence encoding the complete LRF-2 protein in the deposited clone, including the AUG initiation codon and the naturally associated leader sequence shown in SEQ ID NO:4, is amplified using PCR oligonucleotide primers corresponding to the 5′ and 3′ sequences of the gene. The 5′ primer has the same sequence as that above for the integral membrane protein form of LRF-2, while the 3′ primer has the sequence 5′ CGC GGT ACC AAG CTT TCA GCC CCA CAC ATG ACC 3′ (SEQ ID NO:20) containing the underlined Asp718 restriction site followed by 24 nucleotides complementary to a sequence in the 3′ end of the coding sequence in FIGS. 2A, 2B, 2C, and 2D (SEQ ID NO:3) upstream of the transmembrane domain.

For any of the above LRF-1 or LRF-2 constructs, the amplified fragment is isolated from a 1% agarose gel using a commercially available kit (“Geneclean,” BIO 101 Inc., La Jolla, Calif.). The fragment then is digested with BamHI and Asp17 and again is purified on a 1% agarose gel. This fragment is designated herein F1. The vector plasmid is digested with the restriction enzymes BamHI and Asp17 and optionally, can be dephosphorylated using calf intestinal phosphatase, using routine procedures known in the art. The DNA is then isolated from a 1% agarose gel using a commercially available kit (“Geneclean” BIO 101 Inc., La Jolla, Calif.). This vector DNA is designated herein “V1”.

Fragment F1 and the dephosphorylated plasmid V1 are ligated together with T4 DNA ligase. E. coli HB101 or other suitable E. coli hosts such as XL-1 Blue (Statagene Cloning Systems, La Jolla, Calif.) cells are transformed with the ligation mixture and spread on culture plates. Bacteria are identified that contain the plasmid with the human LRF-1 or LRF-2 gene fragment by digesting DNA from individual colonies using BamHI and Asp 17 and then analyzing the digestion product by gel electrophoresis. The sequence of the cloned fragment is confirmed by DNA sequencing. This plasmid is designated herein pA2LRF-1 or pA2LRF-2c(omplete) or pA2LRF-2e(xtracellular).

Five μg of the plasmid pA2LRF-1 or pA2LRF-2c or pA2LRF-2e is co-transfected with 1.0 μg of a commercially available linearized baculovirus DNA (“BaculoGold™ baculovirus DNA”, Pharmingen, San Diego, Calif.), using the lipofection method described by Felgner et al., Proc. Natl. Acad. Sci. USA 84: 7413-7417 (1987). One μg of BaculoGold™ virus DNA and 5 μg of the plasmid pA2LRF-2 are mixed in a sterile well of a microtiter plate containing 50 μl of serum-free Grace's medium (Life Technologies Inc., Gaithersburg, Md.). Afterwards, 10 μl Lipofectin plus 90 μl Grace's medium are added, mixed and incubated for 15 minutes at room temperature. Then the transfection mixture is added drop-wise to Sf9 insect cells (ATCC CRL 1711) seeded in a 35 mm tissue culture plate with 1 ml Grace's medium without serum. The plate is then incubated for 5 hours at 27° C. The transfection solution is then removed from the plate and 1 ml of Grace's insect medium supplemented with 10% fetal calf serum is added. Cultivation is then continued at 27° C. for four days.

After four days the supernatant is collected and a plaque assay is performed, as described by Summers and Smith, supra. An agarose gel with “Blue Gal” (Life Technologies Inc., Gaithersburg) is used to allow easy identification and isolation of gal-expressing clones, which produce blue-stained plaques. (A detailed description of a “plaque assay” of this type can also be found in the user's guide for insect cell culture and baculovirology distributed by Life Technologies Inc., Gaithersburg, page 9-10). After appropriate incubation, blue stained plaques are picked with the tip of a micropipettor (e.g., Eppendorf). The agar containing the recombinant viruses is then resuspended in a microcentrifuge tube containing 200 μl of Grace's medium and the suspension containing the recombinant baculovirus is used to infect Sf9 cells seeded in 35 mm dishes. Four days later the supernatants of these culture dishes are harvested and then they are stored at 4° C. The recombinant virus is called V-LRF-1 or V-LRF-2c or V-LRF-2c.

To verify the expression of the LRF-1 or LRF-2 gene, Sf9 cells are grown in Grace's medium supplemented with 10% heat-inactivated FBS. The cells are infected with the recombinant V-LRF-1 or V-LRF-2 baculovirus at a multiplicity of infection (“MOI”) of about 2. If radiolabeled proteins are desired, 6 hours later the medium is removed and is replaced with SF900 II medium minus methionine and cysteine (available from Life Technologies Inc., Rockville, Md.). After 42 hours, 5 μCi of ³⁵S-methionine and 5 μCi ³⁵S-cysteine (available from Amersham) are added. The cells are further incubated for 16 hours and then are harvested by centrifugation. The proteins in the supernatant as well as the intracellular proteins (membrane bound proteins, in the case of the integral membrane form of LRF-2 expressed from the complete cDNA of the deposit) are analyzed by SDS-PAGE followed by autoradiography (if radiolabeled).

Microsequencing of the amino acid sequence of the amino terminus of purified protein may be used to determine the amino terminal sequence of the mature form of the LRF-1 or LRF-2 protein and thus the cleavage point and length of the naturally associated secretory signal peptide.

### Example 3

### Cloning and Expression of LRF-1 and LRF-2 in Mammalian Cells

A typical mammalian expression vector contains the promoter element, which mediates the initiation of transcription of mRNA, the protein coding sequence, and signals required for the termination of transcription and polyadenylation of the transcript. Additional elements include enhancers, Kozak sequences and intervening sequences flanked by donor and acceptor sites for RNA splicing. Highly efficient transcription can be achieved with the early and late promoters from SV40, the long terminal repeats (LTRs) from Retroviruses, e.g., RSV, HTLVI, HIVI and the early promoter of the cytomegalovirus (CMV). However, cellular elements can also be used (e.g., the human actin promoter). Suitable expression vectors for use in practicing the present invention include, for example, vectors such as pSVL and pMSG (Pharmacia, Uppsala, Sweden), pRSVcat (ATCC 37152), pSV2dhfr (ATCC 37146) and pBC12MI (ATCC 67109). Mammalian host cells that could be used include, human Hela, 293, H9 and Jurkat cells, mouse NIH3T3 and C127 cells, Cos 1, Cos 7 and CV1, quail QC1-3 cells, mouse L cells and Chinese hamster ovary (CHO) cells.

Alternatively, the gene can be expressed in stable cell lines that contain the gene integrated into a chromosome. The co-transfection with a selectable marker such as dhfr, gpt, neomycin, hygromycin allows the identification and isolation of the transfected cells.

The transfected gene can also be amplified to express large amounts of the encoded protein. The DHFR (dihydrofolate reductase) marker is useful to develop cell lines that carry several hundred or even several thousand copies of the gene of interest. Another useful selection marker is the enzyme glutamine synthase (GS) (Murphy et al., Biochem J. 227:277-279 (1991); Bebbington et al., Bio/Technology 10:169-175 (1992)). Using these markers, the mammalian cells are grown in selective medium and the cells with the highest resistance are selected. These cell lines contain the amplified gene(s) integrated into a chromosome. Chinese hamster ovary (CHO) and NSO cells are often used for the production of proteins.

The expression vectors pC1 and pC4 contain the strong promoter (LTR) of the Rous Sarcoma Virus (Cullen et al, Molecular and Cellular Biology, 438-447 (March, 1985)) plus a fragment of the CMV-enhancer (Boshart et al, Cell 41:521-530 (1985)). Multiple cloning sites, e.g., with the restriction enzyme cleavage sites BamHI, XbaI and Asp718, facilitate the cloning of the gene of interest. The vectors contain in addition the 3′ intron, the polyadenylation and termination signal of the rat preproinsulin gene.

### Example 3(a)

### Cloning and Expression in COS Cells

The expression plasmid, pLRF-1HA or pLRF-1HA, is made by cloning a portion of the cDNA encoding the mature LRF-1 or mature or extracellular form of LRF2 protein into the expression vector pcDNAI/Amp or pcDNAIII (which can be obtained from Invitrogen, Inc.).

The expression vector pcDNAI/amp contains: (1) an E. coli origin of replication effective for propagation in E. coli and other prokaryotic cells; (2) an ampicillin resistance gene for selection of plasmid-containing prokaryotic cells; (3) an SV40 origin of replication for propagation in eukaryotic cells; (4) a CMV promoter, a polylinker, an SV40 intron; (5) several codons encoding a hemagglutinin fragment (i.e., an “HA” tag to facilitate purification) followed by a termination codon and polyadenylation signal arranged so that a cDNA can be conveniently placed under expression control of the CMV promoter and operably linked to the SV40 intron and the polyadenylation signal by means of restriction sites in the polylinker. The HA tag corresponds to an epitope derived from the influenza hemagglutinin protein described by Wilson et al., Cell 37: 767 (1984). The fusion of the HA tag to the target protein allows easy detection and recovery of the recombinant protein with an antibody that recognizes the HA epitope. pcDNAIII contains, in addition, the selectable neomycin marker.

A DNA fragment encoding the desired form of the LRF-1 or LRF-2 polypeptide is cloned into the polylinker region of the vector so that recombinant protein expression is directed by the CMV promoter. The plasmid construction strategy is as follows. The desired portion of the LRF-1 of LRF-2 cDNA of the deposited clone is amplified using primers that contain convenient restriction sites, much as described above for construction of vectors for expression of LRF-1 or LRF-2 in insect cells, including, in the 5′ primer, a Kozak sequence, an AUG start codon, and about 15 to 18 nucleotides of the 5′ coding region of the desired LRF-1 or LRF-2 polypeptide. The 3′ primers described for expression in insect cells, above, also are suitable for the present example.

The PCR amplified DNA fragment and the vector, pcDNAI/Amp, are digested with appropriate restriction enzymes and then ligated. The ligation mixture is transformed into E. coli strain SURE (available from Stratagene Cloning Systems, 11099 North Torrey Pines Road, La Jolla, Calif. 92037), and the transformed culture is plated on ampicillin media plates which then are incubated to allow growth of ampicillin resistant colonies. Plasmid DNA is isolated from resistant colonies and examined by restriction analysis or other means for the presence of the fragment encoding the desired form of the LRF-1 or LRF-2 polypeptide.

For expression of recombinant LRF-1 or LRF-2, COS cells are transfected with an expression vector, as described above, using DEAE-DEXTRAN, as described, for instance, in Sambrook et al., Molecular Cloning: a Laboratory Manual, Cold Spring Laboratory Press, Cold Spring Harbor, N.Y. (1989). Cells are incubated under conditions for expression of LRF-1 or LRF-2 by the vector.

Expression of the LRF-1-HA or LRF-2-HA fusion protein is detected by radiolabeling and immunoprecipitation, using methods described in, for example Harlow et al., Antibodies: A Laboratory Manual, 2nd Ed.; Cold Spring Harbor Laboratory Press, Cold Spring Harbor, N.Y. (1988). To this end, two days after transfection, the cells are labeled by incubation in media containing ³⁵S-cysteine for 8 hours. The cells and the media are collected, and the cells are washed and the lysed with detergent-containing RIPA buffer: 150 mM NaCl, 1% NP-40, 0.1% SDS, 1% NP-40, 0.5% DOC, 50 mM TRIS, pH 7.5, as described by Wilson et al. cited above. Proteins are precipitated from the cell lysate and from the culture media using an HA-specific monoclonal antibody. The precipitated proteins then are analyzed by SDS-PAGE and autoradiography. An expression product of the expected size is seen in the cell lysate, which is not seen in negative controls.

### Example 3(b)

### Cloning and Expression in CHO Cells

The vector pC4 is used for the expression of LRF-1 polypeptide. Plasmid pC4 is a derivative of the plasmid pSV2-dhfr (ATCC Accession No. 37146). The plasmid contains the mouse DHFR gene under control of the SV40 early promoter. Chinese hamster ovary- or other cells lacking dihydrofolate activity that are transfected with these plasmids can be selected by growing the cells in a selective medium (alpha minus MEM, Life Technologies) supplemented with the chemotherapeutic agent methotrexate. The amplification of the DHFR genes in cells resistant to methotrexate (MTX) has been well documented (see, e.g., Alt, F. W., Kellems, R. M., Bertino, J. R., and Schimke, R. T., 1978, J. Biol. Chem. 253:1357-1370, Hamlin, J. L. and Ma, C. 1990, Biochem. et Biophys. Acta, 1097:107-143, Page, M. J. and Sydenham, M. A. 1991, Biotechnology 9:64-68). Cells grown in increasing concentrations of MTX develop resistance to the drug by overproducing the target enzyme, DHFR, as a result of amplification of the DHFR gene. If a second gene is linked to the DHFR gene, it is usually co-amplified and over-expressed. It is known in the art that this approach may be used to develop cell lines carrying more than 1,000 copies of the amplified gene(s). Subsequently, when the methotrexate is withdrawn, cell lines are obtained which contain the amplified gene integrated into one or more chromosome(s) of the host cell.

Plasmid pC4 contains for expressing the gene of interest the strong promoter of the long terminal repeat (LTR) of the Rouse Sarcoma Virus (Cullen, et al., Molecular and Cellular Biology, March 1985:438-447) plus a fragment isolated from the enhancer of the immediate early gene of human cytomegalovirus (CMV) (Boshart et al., Cell 41:521-530 (1985)). Downstream of the promoter are the following single restriction enzyme cleavage sites that allow the integration of the genes: BamHI, XbaI, and Asp718. Behind these cloning sites the plasmid contains the 3′ intron and polyadenylation site of the rat preproinsulin gene. Other high efficiency promoters can also be used for the expression, e.g., the human β-actin promoter, the SV40 early or late promoters or the long terminal repeats from other retroviruses, e.g., HIV and HTLVI. Clontech's Tet-Off and Tet-On gene expression systems and similar systems can be used to express the LRF-1 polypeptide in a regulated way in mammalian cells (Gossen, M., & Bujard, H. 1992, Proc. Natl. Acad. Sci. USA 89:5547-5551). For the polyadenylation of the mRNA other signals, e.g., from the human growth hormone or globin genes can be used as well. Stable cell lines carrying a gene of interest integrated into the chromosomes can also be selected upon co-transfection with a selectable marker such as gpt, G418 or hygromycin. It is advantageous to use more than one selectable marker in the beginning, e.g., G418 plus methotrexate.

The cDNA sequence encoding the full length LRF-1 protein in the deposited clone, including the AUG initiation codon and the naturally associated leader sequence shown in SEQ ID NO:2, is amplified using PCR oligonucleotide primers corresponding to the 5′ and 3′ sequences of the gene. The 5′ primer has the sequence 5′ CGC GGA TCC GCC ATC ATG GCA GTA GGG GGC GTT TTG 3′ (SEQ ID NO: 16) containing the underlined BamHI restriction enzyme site, an efficient signal for initiation of translation in eukaryotic cells, as described by Kozak, M., J. Mol. Biol. 196:947-950 (1987), followed by 18 nucleotides of the sequence of the complete LRF-1 protein shown in FIGS. 1A and 1B, beginning with the AUG initiation codon. The 3′ primer has the sequence 5′ CGC GGT ACC GAA TGT GGC ACA GTG 3′ (SEQ ID NO:17) containing the underlined Asp718 restriction site followed by 15 nucleotides complementary to the 3′ noncoding sequence in FIGS. 2A, 2B, 2C, and 2D (SEQ ID NO:3).

Similarly, for expression of the integral membrane protein form of LRF-2, the cDNA sequence encoding the complete LRF-2 protein in the deposited clone, including the AUG initiation codon and the naturally associated leader sequence shown in SEQ ID NO:4, is amplified using PCR oligonucleotide primers corresponding to the 5′ and 3′ sequences of the gene. The 5′ primer has the sequence 5′ CGC GGA TCC GCC ATC ATG CAC GGC TCC TGC AG 3′ (SEQ ID NO:18) containing the underlined BamHI restriction enzyme site, an efficient signal for initiation of translation in eukaryotic cells, as described by Kozak, M., J. Mol. Biol. 196:947-950 (1987), followed by 17 nucleotides of the sequence of the complete LRF-2 protein shown in FIGS. 2A, 2B, 2C, and 2D, beginning with the AUG initiation codon. The 3′ primer has the sequence 5′ CGC GGT ACC GTC TCT CAC TTG GAG GA 3′ (SEQ ID NO:19) containing the underlined Asp718 restriction site followed by 17 nucleotides complementary to the 3′ noncoding sequence in FIGS. 2A, 2B, 2C, and 2D. For expression of the soluble extracellular domain of the LRF-2 polypeptide, without the transmembrane domain, the extracellular portion of the cDNA sequence encoding the complete LRF-2 protein in the deposited clone, including the AUG initiation codon and the naturally associated leader sequence shown in SEQ ID NO:4, is amplified using PCR oligonucleotide primers corresponding to the 5′ and 3′ sequences of the gene. The 5′ primer has the same sequence as that above for the integral membrane protein form of LRF-2, while the 3′ primer has the sequence 5′ CGC GGT ACC AAG CTT TCA GCC CCA CAC ATG ACC 3′ (SEQ ID NO:20) containing the underlined Asp718 restriction site followed by 24 nucleotides complementary to a sequence in the 3′ end of the coding sequence in FIGS. 2A, 2B, 2C, and 2D upstream of the indicated transmembrane domain.

For any of the above LRF-1 or LRF-2 constructs, the plasmid pC4 is digested with the restriction enzymes BamHI and Asp718 and then dephosphorylated using calf intestinal phosphates by procedures known in the art. The vector is then isolated from a 1% agarose gel. The amplified fragment is digested with the same restriction endonucleases and then purified again on a 1% agarose gel. The isolated fragment and the dephosphorylated vector are then ligated with T4 DNA ligase. E. coli HB 101 or XL-1 Blue cells are then transformed and bacteria are identified that contain the fragment inserted into plasmid pC4 using, for instance, restriction enzyme analysis.

Chinese hamster ovary cells lacking an active DHFR gene are used for transfection. Five μg of the expression plasmid pC4 is cotransfected with 0.5 μg of the plasmid pSVneo using lipofectin (Felgner et al., supra). The plasmid pSV2-neo contains a dominant selectable marker, the neo gene from Tn5 encoding an enzyme that confers resistance to a group of antibiotics including G418. The cells are seeded in alpha minus MEM supplemented with 1 mg/ml G418. After 2 days, the cells are trypsinized and seeded in hybridoma cloning plates (Greiner, Germany) in alpha minus MEM supplemented with 10, 25, or 50 ng/ml of metothrexate plus 1 mg/ml G418. After about 10-14 days single clones are trypsinized and then seeded in 6-well petri dishes or 10 ml flasks using different concentrations of methotrexate (50 nM, 100 nM, 200 nM, 400 nM, 800 nM). Clones growing at the highest concentrations of methotrexate are then transferred to new 6-well plates containing even higher concentrations of methotrexate (1 μM, 2 μM, 5 μM, 10 mM, 20 mM). The same procedure is repeated until clones are obtained which grow at a concentration of 100-200 μM. Expression of the desired gene product is analyzed, for instance, by SDS-PAGE and Western blot or by reversed phase HPLC analysis.

### Example 4

### Tissue Distribution of LRF-1 or LRF-2 mRNA Expression

Northern blot analysis is carried out to examine LRF-1 or LRF-2 gene expression in human tissues, using methods described by, among others, Sambrook et al., cited above. A cDNA probe containing the entire nucleotide sequence of the LRF-1 or LRF-2 protein (SEQ ID NO: 1 or SEQ ID NO:3, respectively) is labeled with ³²P using the rediprime™ DNA labeling system (Amersham Life Science), according to manufacturer's instructions. After labeling, the probe is purified using a CHROMA SPIN-100™ column (Clontech Laboratories, Inc.), according to manufacturer's protocol number PT1200-1. The purified labeled probe is then used to examine various human tissues for LRF-1 or LRF-2 mRNA.

Multiple Tissue Northern (MTN) blots containing various human tissues (H) or human immune system tissues (IM) are obtained from Clontech and are examined with the labeled probe using ExpressHyb™ hybridization solution (Clontech) according to manufacturer's protocol number PT1190-1. Following hybridization and washing, the blots are mounted and exposed to film at −70° C. overnight, and films developed according to standard procedures.

It will be clear that the invention may be practiced otherwise than as particularly described in the foregoing description and examples. Numerous modifications and variations of the present invention are possible in light of the above teachings and, therefore, are within the scope of the appended claims.

The entire disclosure of all publications (including patents, patent applications, journal articles, laboratory manuals, books, or other documents) cited herein are hereby incorporated by reference.

What is claimed is:

1. An isolated nucleic acid molecule nucleic acid molecule comprising a polynucleotide having a nucleotide sequence at least 95% identical to a sequence selected from the group consisting of:
(a) a nucleotide sequence encoding the LRF-1 polypeptide having the complete amino acid sequence in positions −24 to +253 of SEQ ID NO:2;
(b) a nucleotide sequence encoding the predicted mature LRF-1 polypeptide having the amino acid sequence at positions 1-253 in SEQ ID NO:2;
(c) a nucleotide sequence encoding the LRF-1 polypeptide having the complete amino acid sequence excepting the N-terminal methionine encoded by the cDNA clone contained in ATCC Deposit No 97860;
(d) a nucleotide sequence encoding the mature LRF-1 polypeptide having the amino acid sequence encoded by the cDNA clone contained in ATCC Deposit No. 97860; and
(e) a nucleotide sequence complementary to any of the nucleotide sequences in (a), (b), (c) or (d) above.

2. The nucleic acid molecule of claim 1 wherein said polynucleotide has the complete nucleotide sequence in FIGS. 1A-B (SEQ ID NO: 1).

3. The nucleic acid molecule of claim 1 wherein said polynucleotide has the nucleotide sequence in FIGS. 1A-B (SEQ ID NO:1) encoding the LRF-1 polypeptide having the amino acid sequence in positions −24 to 259 of SEQ ID NO:2.

4. The nucleic acid molecule of claim 1 wherein said polynucleotide has the nucleotide sequence in FIGS. 1A-B (SEQ ID NO:1) encoding the predicted mature LRF-1 polypeptide having the amino acid sequence from about 1 to about 253 in SEQ ID NO:2.

5. An isolated nucleic acid molecule comprising a polynucleotide having a nucleotide sequence at least 95% identical to a sequence selected from the group consisting of:
(a) a nucleotide sequence encoding a polypeptide comprising the amino acid sequence of residues n-254 of SEQ ID NO:2, where n is an integer in the range of −24 to +53;
(b) a nucleotide sequence encoding a polypeptide comprising the amino acid sequence of residues −25 to m of SEQ ID NO:2, where m is an integer in the range of +184 to +253;
(c) a nucleotide sequence encoding a polypeptide having the amino acid sequence consisting of residues n-m of SEQ ID NO:2, where n and m are integers as defined respectively in (a) and (b) above; and
(d) a nucleotide sequence encoding a polypeptide consisting of a portion of the complete LRF-1 amino acid sequence encoded by the cDNA clone contained in ATCC Deposit No. 97860 wherein said portion excludes from 1 to about 53 amino acids from the amino terminus of said complete amino acid sequence encoded by the cDNA clone contained in ATCC Deposit No. 97860;
(e) a nucleotide sequence encoding a polypeptide consisting of a portion of the complete LRF-1 amino acid sequence encoded by the cDNA clone contained in ATCC Deposit No. 97860 wherein said portion excludes from 1 to about 69 amino acids from the carboxy terminus of said complete amino acid sequence encoded by the cDNA clone contained in ATCC Deposit No. 97860; and
(f) a nucleotide sequence encoding a polypeptide consisting of a portion of the complete LRF-I amino acid sequence encoded by the cDNA clone contained in ATCC Deposit No. 97860 wherein said portion includes a combination of any of the amino terminal and carboxy terminal deletions in (d) and (e), above.

6. The nucleic acid molecule of claim 1 wherein said polynucleotide has the complete nucleotide sequence of the cDNA clone contained in ATCC Deposit No. 97860.

7. The nucleic acid molecule of claim 1 wherein said polynucleotide has the nucleotide sequence encoding the LRF-1 polypeptide having the complete amino acid sequence excepting the N-terminal methionine encoded by the cDNA clone contained in ATCC Deposit No. 97860.

8. The nucleic acid molecule of claim 1 wherein said polynucleotide has the nucleotide sequence encoding the mature LRF-1 polypeptide having the amino acid sequence encoded by the cDNA clone contained in ATCC Deposit No. 97860.

9. An isolated nucleic acid molecule comprising a polynucleotide which hybridizes under stringent hybridization conditions to a polynucleotide having a nucleotide sequence identical to a nucleotide sequence in (a), (b), (c) or (d) of claim 1 wherein said polynucleotide which hybridizes does not hybridize under stringent hybridization conditions to a polynucleotide having a nucleotide sequence consisting of only A residues or of only T residues.

10. An isolated nucleic acid molecule comprising a polynucleotide which encodes the amino acid sequence of an epitope-bearing portion of a LRF-1 polypeptide having an amino acid sequence in (a), (b), (c) or (d) of claim 1.

11. The isolated nucleic acid molecule of claim 10, which encodes an epitope-bearing portion of a LRF-1 polypeptide wherein the amino acid sequence of said portion is selected from the group of sequences consisting of: about His 19 to about Phe 45 in SEQ ID NO:2; about Ala 97 to about Ile 125 in SEQ ID NO:2; about Gly 154 to about Ile 195 in SEQ ID NO:2; and; about Leu 203 to about Leu 249 in SEQ ID NO:2.

12. A method for making a recombinant vector comprising inserting an isolated nucleic acid molecule of claim 1 into a vector.

13. A recombinant vector produced by the method of claim 12.

14. A method of making a recombinant host cell comprising introducing the recombinant vector of claim 13 into a host cell.

15. A recombinant host cell produced by the method of claim 14.

16. A recombinant method for producing an LRF-1 polypeptide, comprising culturing the recombinant host cell of claim 15 under conditions such that said polypeptide is expressed and recovering said polypeptide.

17. An isolated LRF-1 polypeptide comprising an amino acid sequence at least 95% identical to a sequence selected from the group consisting of:
(a) the amino acid sequence at positions −24 to +253 of SEQ ID NO:2 or the complete LRF-1 amino acid sequence excepting the N-terminal methionine encoded by the cDNA clone contained in ATCC Deposit No. 97860; and
(b) the amino acid sequence of the mature LRF-1 polypeptide having the amino acid sequence at positions +1 to +253 in SEQ ID NO:2, or as encoded by the cDNA clone contained in the ATCC Deposit No. 97860.

18. An isolated polypeptide comprising an epitope-bearing portion of the LRF-1 protein, wherein said portion is selected from the group consisting of: a polypeptide comprising amino acid residues from about His 19 to about Phe 45 in SEQ ID NO:2; about Ala 97 to about Ile 125 in SEQ ID NO:2; about Gly 154 to about Ile 195 in SEQ ID NO:2; and; about Leu 203 to about Leu 249 in SEQ ID NO:2.

19. An isolated antibody that binds specifically to an LRF-1 polypeptide of claim 17.

20. An isolated nucleic acid molecule comprising a polynucleotide having a sequence at least 95% identical to the nucleotide sequence of clone HTEIX55R (SEQ ID NO:7).

21. An isolated nucleic acid molecule comprising a polynucleotide comprising a nucleotide sequence selected from the group consisting of:
(a) a nucleotide sequence encoding the LRF-2 polypeptide having the amino acid sequence in positions −22 to +441 of SEQ ID NO:4;
(b) a nucleotide sequence encoding the predicted mature LRF-2 polypeptide having the amino acid sequence at positions +1 to +441 or at positions +3 to +441 in SEQ ID NO:4;
(c) a nucleotide sequence encoding the extracellular domain of the LRF-2 polypeptide having the amino acid sequence at about position +1 to about position +418 or at about position −2 to about position +418 in SEQ ID NO:4;
(d) a nucleotide sequence encoding the LRF-2 polypeptide having the complete amino acid sequence excepting the N-terminal methionine encoded by the cDNA clone contained in ATCC Deposit No 97867;
(e) a nucleotide sequence encoding the mature LRF-2 polypeptide having the amino acid sequence encoded by the cDNA clone contained in ATCC Deposit No. 97867;
(f) a nucleotide sequence encoding the extracellular domain of the LRF-2 polypeptide having the amino acid sequence of the mature LRF-2 polypeptide encoded by the cDNA clone contained in ATCC Deposit No. 97867 excepting the C-terminal sequence of about 23 amino acids of the mature LRF-2 polypeptide encoded by said cDNA; and
(g) a nucleotide sequence complementary to any of the nucleotide sequences in (a), (b), (c), (d), (e) or (f) above.

22. The nucleic acid molecule of claim 21 wherein said polynucleotide has the complete nucleotide sequence in FIGS. 2A-D (SEQ ID NO:3).

23. The nucleic acid molecule of claim 21 wherein said polynucleotide has the nucleotide sequence in FIGS. 2A-D (SEQ ID NO:3) encoding the LRF-2 polypeptide having the amino acid sequence in positions −22 to +441 of SEQ ID NO:4.

24. The nucleic acid molecule of claim 21 wherein said polynucleotide has the nucleotide sequence in FIGS. 2A-D (SEQ ID NO:3) encoding the predicted mature LRF-2 polypeptide having the amino acid sequence from about +1 to about +441 in SEQ ID NO:4.

25. An isolated nucleic acid molecule comprising a polynucleotide having a nucleotide sequence at least 95% identical to a sequence selected from the group consisting of:
(a) a nucleotide sequence encoding a polypeptide comprising the amino acid sequence of residues n to +441 of SEQ ID NO:4, where n is an integer in the range of −22 to +46;
(b) a nucleotide sequence encoding a polypeptide comprising the amino acid sequence of residues −22 to m of SEQ ID NO:4, where m is an integer in the range of +166 to +441;
(c) a nucleotide sequence encoding a polypeptide having the amino acid sequence consisting of residues n-m of SEQ ID NO:4, where n and m are integers as defined respectively in (a) and (b) above; and
(d) a nucleotide sequence encoding a polypeptide consisting of a portion of the complete LRF-2 amino acid sequence encoded by the cDNA clone contained in ATCC Deposit No. 97867 wherein said portion excludes from 1 to about 53 amino acids from the amino terminus of said complete amino acid sequence encoded by the cDNA clone contained in ATCC Deposit No.97867;
(e) a nucleotide sequence encoding a polypeptide consisting of a portion of the complete LRF-2 amino acid sequence encoded by the cDNA clone contained in ATCC Deposit No. 97867 wherein said portion excludes from 1 to about 277 amino acids from the carboxy terminus of said complete amino acid sequence encoded by the cDNA clone contained in ATCC Deposit No.97867; and
(f) a nucleotide sequence encoding a polypeptide consisting of a portion of the complete LRF-2 amino acid sequence encoded by the cDNA clone contained in ATCC Deposit No. 97867 wherein said portion includes a combination of any of the amino terminal and carboxy terminal deletions in (d) and (e), above.

26. The nucleic acid molecule of claim 21 wherein said polynucleotide has the complete nucleotide sequence of the cDNA clone contained in ATCC Deposit No. 97867.

27. The nucleic acid molecule of claim 21 wherein said polynucleotide has the nucleotide sequence encoding the LRF-2 polypeptide having the complete amino acid sequence excepting the N-terminal methionine encoded by the cDNA clone contained in ATCC Deposit No. 97867.

28. The nucleic acid molecule of claim 21 wherein said polynucleotide has the nucleotide sequence encoding the mature LRF-2 polypeptide having the amino acid sequence encoded by the cDNA clone contained in ATCC Deposit No. 97867.

29. An isolated nucleic acid molecule comprising a polynucleotide which hybridizes under stringent hybridization conditions to a polynucleotide having a nucleotide sequence identical to a nucleotide sequence in (a), (b), (c), (d), (e) or (f) of claim 21 wherein said polynucleotide which hybridizes does not hybridize under stringent hybridization conditions to a polynucleotide having a nucleotide sequence consisting of only A residues or of only T residues.

30. An isolated nucleic acid molecule comprising a polynucleotide which encodes the amino acid sequence of an epitope-bearing portion of an LRF-2 polypeptide having an amino acid sequence in (a), (b), (c), (d), (e) or (f) of claim 21.

31. The isolated nucleic acid molecule of claim 30, which encodes an epitope-bearing portion of an LRF-2 polypeptide wherein the amino acid sequence of said portion is selected from the group of sequences consisting of: about His 58 to about Asn 68 of SEQ ID NO:4; about Glu 84 to about Ser 96 of SEQ ID NO:4; about Glu 146 to about Pro 162 of SEQ ID NO:4; about Pro 296 to about Lys 320 of SEQ ID NO:4; about Arg 180 to about Thr 212 in SEQ ID NO:2; about Glu 241 to about Glu 259 of SEQ ID NO:4; about His 275 to about His 292 in SEQ ID NO:2; about Pro 354 to about Pro 365 in SEQ ID NO:2; about His 372 to about Thr 387 of SEQ ID NO:4; and about Ser 400 to about Ser 415 of SEQ ID NO:4.

32. A method for making a recombinant vector comprising inserting an isolated nucleic acid molecule of claim 21 into a vector.

33. A recombinant vector produced by the method of claim 32.

34. A method of making a recombinant host cell comprising introducing the recombinant vector of claim 33 into a host cell.

35. A recombinant host cell produced by the method of claim 34.

36. A recombinant method for producing an LRF-2 polypeptide, comprising culturing the recombinant host cell of claim 35 under conditions such that said polypeptide is expressed and recovering said polypeptide.

37. An isolated LRF-2 polypeptide comprising an amino acid sequence at least 95% identical to a sequence selected from the group consisting of:
(a) the amino acid sequence in positions −19 to +443 of SEQ ID NO:4;
(b) the amino acid sequence at positions +1 to +443 or at positions +3 to +443 of SEQ ID NO:4;
(c) the amino acid sequence at about position +1 to about position +421 or at about position +3 to about position +421 of SEQ ID NO:4;
(d) the amino acid sequence of the LRF-2 polypeptide having the complete amino acid sequence excepting the N-terminal methionine encoded by the cDNA clone contained in ATCC Deposit No 97867;
(e) the amino acid sequence of the mature LRF-2 polypeptide having the amino acid sequence encoded by the cDNA clone contained in ATCC Deposit No. 97867; and
(f) the amino acid sequence of the extracellular domain of the LRF-2 polypeptide having the amino acid sequence of the mature LRF-2 polypeptide encoded by the cDNA clone contained in ATCC Deposit No. 97867 excepting the C-terminal sequence of about 23 amino acids of the mature LRF-2 polypeptide encoded by that cDNA.

38. An isolated polypeptide comprising an epitope-bearing portion of the LRF-2 protein, wherein said portion is selected from the group consisting of: about His 58 to about Asn 68 of SEQ ID NO:4; about Glu 84 to about Ser 96 of SEQ ID NO:4; about Glu 146 to about Pro 162 of SEQ ID NO:4; about Pro 296 to about Lys 320 of SEQ ID NO:4; about Arg 180 to about Thr 212 in SEQ ID NO:2; about Glu 241 to about Glu 259 of SEQ ID NO:4; about His 275 to about His 292 in SEQ ID NO:2; about Pro 354 to about Pro 365 in SEQ ID NO:2; about His 372 to about Thr 387 of SEQ ID NO:4; and about Ser 400 to about Ser 415 of SEQ ID NO:4.

39. An isolated antibody that binds specifically to an LRF-2 polypeptide of claim 37.

40. An isolated nucleic acid molecule comprising a polynucleotide having a sequence at least 95% identical to the nucleotide sequence of clone selected from the group consisting of HJAAR51 (SEQ ID NO:8); HARAZ76 (SEQ ID NO:9); HRDBF59 (SEQ ID NO:10); and HJABC86 (SEQ ID NO:11).
